(19) United States
(12) Patent Application Publication
(10) Pub. No.: US 2023/0142529 A1
(43) Pub. Date: May 11, 2023

(54) RNA FORMULATIONS FOR HIGH VOLUME DISTRIBUTION

(71) Applicant: ModernaTX, Inc., Cambridge, MA (US)

(72) Inventors: Philip White, Burlington, MA (US); Jack F. Kramarczyk, Somerville, MA (US); Julia O'Neill, Glenside, PA (US); Nedim Emil Altaras, Cambridge, MA (US)

(73) Assignee: ModernaTX, Inc., Cambridge, MA (US)

(21) Appl. No.: 17/925,114

(22) PCT Filed: May 14, 2021
(86) PCT No.: PCT/US2021/032555

§ 371 Date: Nov. 14, 2022

Related U.S. Application Data
(60) Provisional application No. 63/030,739, filed on May 27, 2020.
(60) Provisional application No. 63/025,936, filed on May 15, 2020.

(51) Int. Cl. C12N 15/88; A61K 31/713

(52) U.S. Cl. CPC C12N 15/88; A61K 31/713; A61K 9/127

(57) ABSTRACT

Present application relates to a strategy for compensating for transesterification degradation of lipid-encapsulated RNA, such as mRNA-LNP, in liquid formulations for high-volume distribution. This involves determining the rate of degradation of the encapsulated RNA and calculating an appropriate overage relative to the intended dose. Alternatively, a higher dose of the RNA may be administered to compensate for loss of effective RNA or the RNA may be formulated in higher purity in anticipation of degradation. The strategy provides a balance between supplying effective and safe products and the need for costly manufacturing processes or transportation hurdles, such as cold-chain supply.

### RELATED APPLICATIONS

This application claims priority under 35 U.S.C. § 119(e) to U.S. Provisional Patent Application No. 63/025,936, filed May 15, 2020 and U.S. Provisional Patent Application No. 63/030,739, filed May 27, 2020, which are hereby incorporated by reference in their entireties.

### FIELD OF INVENTION

The present disclosures relate generally to formulations of nucleic acids (e.g., mRNA) formulated in lipid carriers (e.g., lipid nanoparticles (LNPs)), and more specifically to articles suitable for high volume distribution that comprise formulations comprising nucleic acids (e.g., mRNA) formulated in lipid carriers (e.g., LNPs), and related methods of preparing and using the same.

### BACKGROUND

The use of messenger RNA as a pharmaceutical agent is of great interest for a variety of applications, including in therapeutics, vaccines and diagnostics. Effective in vivo delivery of mRNA formulations represents a continuing challenge, as many such formulations are inherently unstable, activate an immune response, are susceptible to degradation by nucleases, or fail to reach their target organs or cells within the body due to issues with biodistribution. Each of these challenges results in loss of translational potency and therefore hinders efficacy of conventional mRNA pharmaceutical agents.

Various non-viral delivery systems, including nanoparticle formulations, present attractive opportunities to overcome many challenges associated with mRNA delivery. In particular, lipid nanoparticles (LNPs) have drawn particular attention in recent years as various LNP formulations have shown promise in a variety of pharmaceutical applications (Kowalski et al., “Delivering the Messenger: Advances in Technologies for Therapeutic mRNA Delivery” Molecular Therapy, 27(4):710-728 (2019); Gómez-Aguado, et al., “Nanomedicines to Deliver mRNA: State of the Art and Future Perspectives” Nanomaterials, 10, 264 (2020); Wadhwa et al., “Opportunities and Challenges in the Delivery of mRNA-Based Vaccines” Pharmaceutics, 12, 102 (2020)).

### SUMMARY OF THE INVENTION

The present invention provides, among other things, articles (e.g., articles suitable for high volume distribution, including, for instance, distribution of vials comprising various amounts of intact, full length RNA, including different amounts at different times during storage, transportation and shelf life and distribution of individual doses comprising various amounts of intact, full length RNA) comprising liquid pharmaceutical compositions comprising a nucleic acid (e.g., RNA, such as mRNA) formulated in a lipid carrier (e.g., LNP), and methods of preparing and using the same. The invention encompasses, in some aspects, the determination of the degradation rate of RNA (e.g., mRNA) and the determination of the appropriate balance between the degradation rate and other relevant factors (e.g., complexity of manufacturing, cost of manufacturing, volume of manufacturing, and/or usefulness of the product globally) in the context of high volume distribution.

According to some aspects, articles are provided herein.

In certain embodiments, the article comprises a liquid pharmaceutical composition comprising RNA formulated in a lipid nanoparticle, liposome, or lipoplex; and a label, suggesting an amount of the liquid pharmaceutical composition to be administered to a subject; wherein the article has a shelf-life of at least three months when stored at a temperature of greater than 0° C. and less than or equal to 10° C.; and wherein the amount is greater than or equal to (1+the fraction of the RNA that would degrade in the liquid pharmaceutical composition over the shelf-life of the article)×(an individual dose of the liquid pharmaceutical composition). In some embodiments, the article comprises a total amount of full length RNA, and the total amount of full length RNA is greater than or equal to (1+the fraction of the full length RNA that would degrade in the liquid pharmaceutical composition over the shelf-life of the article)×(an individual dose of the full length RNA)×(the number of individual doses of the liquid pharmaceutical composition in the article).

In certain embodiments, the article comprises a liquid pharmaceutical composition comprising RNA formulated in a lipid nanoparticle, liposome, or lipoplex; wherein the article has a shelf-life of at least three months when stored at a temperature of greater than 0° C. and less than or equal to 10° C.; and wherein the article comprises a total amount of full length RNA, and the total amount of full length RNA is greater than or equal to (1+the fraction of the full length RNA that would degrade in the liquid pharmaceutical composition over the shelf-life of the article)×(an individual dose of the full length RNA)×(the number of individual doses of the liquid pharmaceutical composition in the article).

In some embodiments, the article comprises a vial, a syringe, a cartridge, an infusion pump, and/or a light protective container.

In certain embodiments, the amount is greater than or equal to 1.05×(an individual dose of the liquid pharmaceutical composition), such as greater than or equal to 1.2×(an individual dose of the liquid pharmaceutical composition).

In some embodiments, the RNA is encapsulated within the lipid nanoparticle, liposome, or lipoplex. In certain embodiments, the lipid nanoparticle, liposome, or lipoplex comprises a lipid nanoparticle.

In certain embodiments, the article comprises a liquid pharmaceutical composition comprising an RNA encoding an antigen formulated in a lipid carrier housed in a container; wherein the container comprises a total amount of RNA and wherein the total amount of RNA includes 40%-95% intact RNA and 5%-60% RNA that is less than full length RNA. In some embodiments, the percentage of intact RNA is greater than or equal to 15%+the percentage of intact RNA that would degrade in the liquid pharmaceutical composition over a shelf-life of the article. In certain embodiments, the article comprises at least 5% more intact RNA than an effective dose of the intact RNA.

In some embodiments, the article comprises a liquid pharmaceutical composition comprising an RNA formulated in a lipid carrier housed in a container; and a label on the container, wherein the label identifies a number of individual doses of the liquid pharmaceutical composition housed in the container, an amount of each individual dose of the liquid pharmaceutical composition to be administered to a subject, and an effective dose of RNA within the liquid pharmaceutical composition within each individual dose, wherein the container comprises a total amount of RNA, wherein the total amount of RNA has a value of at least the number of individual doses in the container times 5% greater than the amount of the effective dose of RNA within each individual dose. In certain embodiments, the container comprises a total amount of full length RNA, wherein the total amount of full length RNA is at least the number of individual doses in the container times 5% greater than the amount of the effective dose of full length RNA within each individual dose.

In certain embodiments, the article has a shelf-life of at least three months when stored at a temperature of greater than 0° C. and less than or equal to 10° C.

In some embodiments, the RNA is encapsulated within the lipid carrier. In certain embodiments, the lipid carrier comprises a lipid nanoparticle.

In certain embodiments, the RNA comprises mRNA. In some embodiments, the RNA comprises greater than or equal to 400, 500, 1000, 2000, 3000, 4000, 5000, 6000, 7000, or 8000 nucleotides. In certain embodiments, the RNA comprises less than or equal to 15,000, 14,000, 13,000, 12,000, 11,000, 10,000, 9000, 8000, 7000, or 6000 nucleotides.

In some embodiments, the liquid pharmaceutical composition is formulated in an aqueous solution. In certain embodiments, the liquid pharmaceutical composition is any pharmaceutical composition disclosed herein.

According to some aspects, pharmaceutical compositions are described herein.

In certain embodiments, the pharmaceutical composition comprises mRNA encapsulated in a lipid nanoparticle, wherein the composition comprises a total amount of intact mRNA that is greater than an effective amount of intact mRNA, and wherein the composition comprises at least the effective amount of the intact mRNA after storage of the composition for a period of time.

In some embodiments, the total amount of intact mRNA decreases in the composition after storage of the composition for the period of time. In certain embodiments, the total amount of intact mRNA is calculated to account for degradation of the intact mRNA during the storage of the composition for the period of time. In some embodiments, the degradation is from transesterification of the intact mRNA. In certain embodiments, the degradation is greater than or equal to 5%, greater than or equal to 7%, greater than or equal to 8%, greater than or equal to 9%, greater than or equal to 10%, or greater than or equal to 12% of the total mRNA in the composition per month.

In certain embodiments, the period of time is greater than or equal to 1 month, greater than or equal to 2 months, greater than or equal to 3 months, greater than or equal to 6 months, or greater than or equal to 9 months. In some embodiments, the storage is at a temperature of from about 0° C. to about 10° C., such as at about 5° C.

In some embodiments, the total amount of intact mRNA is at least 40%, such as at least 50%, at least 55%, at least 60%, at least 63%, at least 65%, at least 70%, at least 75%, at least 80%, at least 85%, at least 90%, or at least 95% of the total mRNA in the composition. In certain embodiments, the effective amount of intact mRNA is at least about 15%, such as at least about 18%, at least about 20%, at least about 25%, at least about 30%, at least about 35%, at least about 40%, at least about 45%, at least about 50%, or at least about 55% of the total mRNA in the composition. In some embodiments, the pharmaceutical composition comprises at least 50% intact mRNA of the total mRNA in the composition following storage of the composition for 3 months at about 5° C. In certain embodiments, the effective amount of intact mRNA comprises at least 5 micrograms of the intact mRNA, such as at least 10 micrograms, at least 20 micrograms, at least 30 micrograms, at least 40 micrograms, at least 50 micrograms, at least 60 micrograms, at least 70 micrograms, at least 80 micrograms, at least 90 micrograms, at least 100 micrograms, at least 125 micrograms, or at least 150 micrograms of the intact mRNA.

According to some aspects, containers are described herein.

In certain embodiments, the container (such as a vial, a syringe, a cartridge, an infusion pump, and/or a light protective container) comprises a pharmaceutical composition disclosed herein.

According to some aspects, methods of filling an article are described herein.

In some embodiments, the method of filling the article comprises adding RNA formulated in a lipid nanoparticle, liposome, or lipoplex to the article to form an amount of a liquid pharmaceutical composition in the article; wherein the amount is greater than or equal to (1+the fraction of the RNA that would degrade in the liquid pharmaceutical composition over the shelf-life of the article)×(an individual dose of the liquid pharmaceutical composition)×(the number of individual doses in the article).

In certain embodiments, the adding RNA formulated in a lipid nanoparticle, liposome, or lipoplex to the article forms an amount of full length RNA in the article, and wherein the amount of full length RNA is greater than or equal to (1+the fraction of the full length RNA that would degrade in the liquid pharmaceutical composition over the shelf-life of the article)×(an individual dose of the full length RNA)×(the number of individual doses in the article).

In certain embodiments, the lipid nanoparticle, liposome, or lipoplex comprises a lipid nanoparticle. In some embodiments, the RNA and/or lipid nanoparticle are frozen prior to addition to the article.

In certain embodiments, the article is stored at a temperature of greater than 0° C. and less than 10° C. for up to 1 year.

In some embodiments, at least 40% of the amount of the RNA in the liquid pharmaceutical composition is intact if stored for three months at a temperature of greater than 0° C. and less than 10° C.

In certain embodiments, the liquid pharmaceutical composition comprises any pharmaceutical composition disclosed herein.

According to some aspects, methods of delivering an effective dose of an RNA to a subject are described herein.

In some embodiments, the method of delivering an effective dose of an RNA to a subject comprises administering a liquid pharmaceutical composition comprising an RNA encoding a protein formulated in a lipid carrier to a subject, wherein a total dose of the RNA is administered to the subject, and wherein the total dose of RNA administered to the subject is at least 5% greater than an effective dose of the RNA. In certain embodiments, the lipid carrier comprises a lipid nanoparticle.

According to some aspects, methods of compensating for transesterification of mRNA in a composition the mRNA encapsulated by a lipid nanoparticle are described herein.

In certain embodiments, the method of compensating for transesterification of mRNA in a composition comprising the mRNA encapsulated by a lipid nanoparticle comprises preparing the composition with increased mRNA purity as compared to an mRNA purity that will be present in the composition after storage of the composition, such that the amount of mRNA present in the composition after storage will comprise an effective amount of the mRNA. In some embodiments, the composition comprises any pharmaceutical composition disclosed herein.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1A shows the mechanism of transesterification in RNA (e.g., mRNA).

FIG. 1B shows the mechanism of hydrolysis in RNA (e.g., mRNA).

FIG. 2A plots the relative abundance of sequence reads versus the position of RNA 3′-terminal nucleotides for liquid mRNA that encodes a viral antigen at 5° C., with and without PNK.

FIG. 2B plots the relative abundance of sequence reads versus the position of RNA 3′-terminal nucleotides for liquid mRNA that encodes a viral antigen at 5° C., with and without PNK.

FIG. 3 plots the % main peak area (normalized to T=0) versus the number of days stored at 40° C. as determined by a size-based RP-HPLC purity method for mRNAs of different lengths.

FIG. 4 plots normalized purity versus time (in months) of an LNP formulation comprising an mRNA that encodes for an antigen when stored at −70° C. or 5° C.

FIG. 5 plots the geometric mean titer produced in vivo versus the percentage purity of the mRNA administered.

FIG. 6 shows a model of stability when a product is stored at −70° C. and then transitioned to 5° C. storage, in accordance with certain embodiments. The dotted line indicates a minimum effective dose, in certain instances. FIG. 6 demonstrates that if additional product is included above the minimum effective dose, the product may be stored at 5° C. for 3 months while still retaining a minimum effective dose, in some cases.

FIG. 7 shows the projected mRNA purity at the time of administration of 15,000 doses of a vaccine.

### DETAILED DESCRIPTION

Lipid nanoparticle (LNP) formulations offer the opportunity to deliver various nucleic acids (e.g., mRNA) in vivo for applications in which unencapsulated nucleic acids would be ineffective. However, nucleic acids (e.g., mRNA) within LNP formulations typically degrade over time (e.g., from trans-esterification). This can be problematic for many applications. For example, in the case of vaccines, if the active agent degrades, an insufficient dose may be administered to a subject, such that the subject may not actually be protected by the vaccine.

Although this degradation may be reduced, in some cases, by lyophilization of the formulation, or by freezing (e.g., at −20° C. or −70° C.), such that the formulations may be stored longer term, these options are not always feasible. For example, not all countries have sufficient cold-chain storage and supply. Accordingly, if a drug is needed throughout the world, freezing the formulations may not be an option for these countries. In fact, even in countries where cold-chain storage and supply is not typically an issue, it might be difficult to have sufficient cold-chain storage and supply if a large volume of formulations are needed. Similarly, the use of lyophilization may complicate manufacturing, increase cost of manufacturing, and/or cause a bottleneck in the supply chain. Still further, refrigerated liquid products are preferred over reconstituted lyophilized powder or frozen products for widespread use as they are more patient-friendly. Accordingly, alternatives are needed for high volume distribution (e.g., distribution globally and/or high volume distribution locally).

Additionally, long term storage can be less important than these other factors when high volume distribution is needed. For example, in a global pandemic, long term storage for a vaccine is less important than the ability to manufacture and distribute large volumes of vaccine. This is because vaccines will not sit on shelves for long periods of time, as vaccines will be needed almost as, or more, quickly than they can be produced. Accordingly, the focus in situations such as this shifts to how rapidly and inexpensively the vaccines can be produced and distributed, rather than on how long they can be stored. Thus, factors such as simplifying manufacturing, decreasing cost, and preventing a bottleneck in the supply chain, as well as the ability to distribute the vaccine globally, become increasingly important.

Nevertheless, the focus cannot exclusively be on rapid production, and long term storage of a formulation cannot be ignored entirely, as it is not always practically feasible for a vaccine to be distributed and used immediately after production. Accordingly, even in times of high volume distribution, a vaccine still must have at least a minimum shelf-life (e.g., three months). The inventors of the present application were able to develop articles and methods that appropriately balance these factors. In some embodiments, the articles and methods disclosed herein provide advantages such as rapid production, simple manufacturing, inexpensive manufacturing, inexpensive storage, and/or accessible storage options, while still ensuring that an effective dose will be delivered to the subject. In certain embodiments, the articles and methods disclosed herein provide advantages such as the capability of high volume production and/or distribution.

In some embodiments, high volume (e.g., production, distribution, and/or administration) comprises greater than or equal to 10 million articles/month, greater than or equal to 25 million articles/month, greater than or equal to 50 million articles/month, greater than or equal to 100 million articles/month, greater than or equal to 150 million articles/month, greater than or equal to 200 million articles/month, or greater than or equal to 250 million articles/month. In certain embodiments, high volume comprises less than or equal to 1 billion articles/month, less than or equal to 500 million articles/month, less than or equal to 250 million articles/month, less than or equal to 200 million articles/month, or less than or equal to 150 million articles/month. Combinations of these ranges are also possible (e.g., greater than or equal to 10 million articles/month and less than or equal to 1 billion articles/month).

In some embodiments, articles (e.g., vials) comprise additional pharmaceutical composition (e.g., additional RNA, such as mRNA, i.e., intact (full length) mRNA) than that required for the number of individual doses contained therein, providing more flexibility in storage conditions (e.g., allowing storing of a liquid pharmaceutical composition at 5° C. for 3 months), as 100% of the RNA (e.g., mRNA) in the article need not be intact to deliver a therapeutically effective dose. In some instances, this flexibility in storage conditions provides advantages such as rapid production, simple manufacturing, inexpensive manufacturing, inexpensive storage, and/or accessible storage options.

Accordingly, provided herein are articles (e.g., articles comprising liquid pharmaceutical compositions) and methods for their preparation and use.

In some embodiments, the article and/or liquid pharmaceutical composition comprises a nucleic acid (e.g., mRNA).

As disclosed herein, the term “nucleic acid” refers to multiple nucleotides (i.e., molecules comprising a sugar (e.g., ribose or deoxyribose) linked to a phosphate group and to an exchangeable organic base, which is either a substituted pyrimidine (e.g., cytosine (C), thymine (T) or uracil (U)) or a substituted purine (e.g., adenine (A) or guanine (G))). As used herein, the term nucleic acid refers to polyribonucleotides as well as polydeoxyribonucleotides. The term nucleic acid shall also include polynucleosides (i.e., a polynucleotide minus the phosphate) and any other organic base containing polymer. Non-limiting examples of nucleic acids include chromosomes, genomic loci, genes or gene segments that encode polynucleotides or polypeptides, coding sequences, non-coding sequences (e.g., intron, 5′-UTR, or 3′-UTR) of a gene, pri-mRNA, pre-mRNA, cDNA, mRNA, etc. In some embodiments, the nucleic acid is mRNA. A nucleic acid may include a substitution and/or modification. In some embodiments, the substitution and/or modification is in one or more bases and/or sugars. For example, in some embodiments a nucleic acid includes nucleic acids having backbone sugars that are covalently attached to low molecular weight organic groups other than a hydroxyl group at the 2′ position and other than a phosphate group or hydroxy group at the 5′ position. Thus, in some embodiments, a substituted or modified nucleic acid includes a 2′-O-alkylated ribose group. In some embodiments, a modified nucleic acid includes sugars such as hexose, 2′-F hexose, 2′-amino ribose, constrained ethyl (cEt), locked nucleic acid (LNA), arabinose or 2′-fluoroarabinose instead of ribose. Thus, in some embodiments, a nucleic acid is heterogeneous in backbone composition thereby containing any possible combination of polymer units linked together such as peptide-nucleic acids (which have an amino acid backbone with nucleic acid bases).

In some embodiments, a nucleic acid is DNA, RNA, PNA, cEt, LNA, ENA or hybrids including any chemical or natural modification thereof. Chemical and natural modifications are well known in the art. Non-limiting examples of modifications include modifications designed to increase translation of the nucleic acid, to increase cell penetration or sub-cellular distribution of the nucleic acid, to stabilize the nucleic acid against nucleases and other enzymes that degrade or interfere with the structure or activity of the nucleic acid, and to improve the pharmacokinetic properties of the nucleic acid.

In some embodiments, the compositions of the present disclosure comprise a RNA having an open reading frame (ORF) encoding a polypeptide. In some embodiments, the RNA is a messenger RNA (mRNA). In some embodiments, the RNA (e.g., mRNA) further comprises a 5′ UTR, 3? UTR, a poly(A) tail and/or a 5′ cap analog.

Messenger RNA (mRNA) is any RNA that encodes a (at least one) protein (a naturally-occurring, non-naturally-occurring, or modified polymer of amino acids) and can be translated to produce the encoded protein in vitro, in vivo, in situ, or ex vivo. The skilled artisan will appreciate that, except where otherwise noted, nucleic acid sequences set forth in the instant application may recite “T”s in a representative DNA sequence but where the sequence represents RNA (e.g., mRNA), the “T”s would be substituted for “U”s. Thus, any of the DNAs disclosed and identified by a particular sequence identification number herein also disclose the corresponding RNA (e.g., mRNA) sequence complementary to the DNA, where each “T” of the DNA sequence is substituted with “U.”

An open reading frame (ORF) is a continuous stretch of DNA or RNA beginning with a start codon (e.g., methionine (ATG or AUG)) and ending with a stop codon (e.g., TAA, TAG or TGA, or UAA, UAG or UGA). An ORF typically encodes a protein. It will be understood that the sequences disclosed herein may further comprise additional elements, e.g., 5′ and 3′ UTRs, but that those elements, unlike the ORF, need not necessarily be present in an RNA polynucleotide of the present disclosure.

Naturally-occurring eukaryotic mRNA molecules can contain stabilizing elements, including, but not limited to untranslated regions (UTR) at their 5′-end (5′ UTR) and/or at their 3′-end (3′ UTR), in addition to other structural features, such as a 5′-cap structure or a 3′-poly(A) tail. Both the 5′ UTR and the 3′ UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing.

In some embodiments, a composition includes an RNA polynucleotide having an open reading frame encoding at least one polypeptide having at least one modification, at least one 5′ terminal cap, and is formulated within a lipid nanoparticle. 5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap]; G(5′)ppp(5′)A; G(5′)ppp(5′)G; m7G(5′)ppp(5′)A; m7G(5′)ppp(5′)G (New England BioLabs, Ipswich, Mass.). 5′-capping of modified RNA may be completed post-transcriptionally using a Vaccinia Virus Capping Enzyme to generate the “Cap 0” structure: m7G(5′)ppp(5′)G (New England BioLabs, Ipswich, Mass.). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes may be derived from a recombinant source.

The 3′-poly(A) tail is typically a stretch of adenine nucleotides added to the 3′-end of the transcribed mRNA. It can, in some instances, comprise up to about 400 adenine nucleotides. In some embodiments, the length of the 3′-poly(A) tail may be an essential element with respect to the stability of the individual mRNA.

In some embodiments, a composition comprises an RNA (e.g., mRNA) having an ORF that encodes a signal peptide fused to the expressed polypeptide. Signal peptides, comprising the N-terminal 15-60 amino acids of proteins, are typically needed for the translocation across the membrane on the secretory pathway and, thus, universally control the entry of most proteins both in eukaryotes and prokaryotes to the secretory pathway. A signal peptide may have a length of 15-60 amino acids.

In some embodiments, an ORF encoding a polypeptide is codon optimized. Codon optimization methods are known in the art. For example, an ORF of any one or more of the sequences provided herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park Calif.) and/or proprietary methods. In some embodiments, the open reading frame (ORF) sequence is optimized using optimization algorithms.

In some embodiments, an RNA (e.g., mRNA) is not chemically modified and comprises the standard ribonucleotides consisting of adenosine, guanosine, cytosine and uridine. In some embodiments, nucleotides and nucleosides of the present disclosure comprise standard nucleoside residues such as those present in transcribed RNA (e.g. A, G, C, or U). In some embodiments, nucleotides and nucleosides of the present disclosure comprise standard deoxyribonucleosides such as those present in DNA (e.g. dA, dG, dC, or dT).

The compositions of the present disclosure comprise, in some embodiments, an RNA having an open reading frame encoding a polypeptide, wherein the nucleic acid comprises nucleotides and/or nucleosides that can be standard (unmodified) or modified as is known in the art. In some embodiments, nucleotides and nucleosides of the present disclosure comprise modified nucleotides or nucleosides. Such modified nucleotides and nucleosides can be naturally-occurring modified nucleotides and nucleosides or non-naturally occurring modified nucleotides and nucleosides. Such modifications can include those at the sugar, backbone, or nucleobase portion of the nucleotide and/or nucleoside as are recognized in the art.

In some embodiments, a naturally-occurring modified nucleotide or nucleotide of the disclosure is one as is generally known or recognized in the art. Non-limiting examples of such naturally occurring modified nucleotides and nucleotides can be found, inter alia, in the widely recognized MODOMICS database.

The present disclosure provides for modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

In some embodiments, modified nucleobases in nucleic acids (e.g., RNA nucleic acids, such as mRNA nucleic acids) comprise 1-methyl-pseudouridine (mlyψ), 1-ethyl-pseudouridine (e1ψ), 5-methoxy-uridine (mo5U), 5-methyl-cytidine (m5C), and/or pseudouridine (ψ). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, a mRNA of the disclosure comprises 1-methyl-pseudouridine (m1ψ) substitutions at one or more or all uridine positions of the nucleic acid.

In some embodiments, a mRNA of the disclosure comprises 1-methyl-pseudouridine (m1ψ) substitutions at one or more or all uridine positions of the nucleic acid and 5-methyl cytidine substitutions at one or more or all cytidine positions of the nucleic acid.

In some embodiments, a mRNA of the disclosure comprises pseudouridine (ψ) substitutions at one or more or all uridine positions of the nucleic acid.

In some embodiments, a mRNA of the disclosure comprises pseudouridine (ψ) substitutions at one or more or all uridine positions of the nucleic acid and 5-methyl cytidine substitutions at one or more or all cytidine positions of the nucleic acid.

In some embodiments, a mRNA of the disclosure comprises uridine at one or more or all uridine positions of the nucleic acid.

In some embodiments, mRNAs are uniformly modified (e.g., fully modified, modified throughout the entire sequence) for a particular modification. For example, a nucleic acid can be uniformly modified with 1-methyl-pseudouridine, meaning that all uridine residues in the mRNA sequence are replaced with 1-methyl-pseudouridine. Similarly, a nucleic acid can be uniformly modified for any type of nucleoside residue present in the sequence by replacement with a modified residue such as those set forth above.

The nucleic acids of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the poly(A) tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The mRNAs of the present disclosure may comprise one or more regions or parts which act or function as an untranslated region. Where mRNAs are designed to encode at least one polypeptide of interest, the nucleic may comprise one or more of these untranslated regions (UTRs). Wild-type untranslated regions of a nucleic acid are transcribed but not translated. In mRNA, the 5′ UTR starts at the transcription start site and continues to the start codon but does not include the start codon; whereas, the 3′ UTR starts immediately following the stop codon and continues until the transcriptional termination signal. The regulatory features of a UTR can be incorporated into the polynucleotides of the present disclosure to, among other things, enhance the stability of the molecule. The specific features can also be incorporated to ensure controlled down-regulation of the transcript in case they are misdirected to undesired organs sites. A variety of 5′UTR and 3′UTR sequences are known and available in the art.

In some embodiments, the nucleic acid (e.g., RNA, such as mRNA) comprises greater than or equal to 400 nucleotides, greater than or equal to 500 nucleotides, greater than or equal to 600 nucleotides, greater than or equal to 800 nucleotides, greater than or equal to 1,000 nucleotides, greater than or equal to 1,500 nucleotides, greater than or equal to 2,000 nucleotides, greater than or equal to 3,000 nucleotides, greater than or equal to 4,000 nucleotides, greater than or equal to 5,000 nucleotides, greater than or equal to 6,000 nucleotides, greater than or equal to 7,000 nucleotides, greater than or equal to 8,000 nucleotides, greater than or equal to 9,000 nucleotides, or greater than or equal to 10,000 nucleotides, greater than or equal to 11,000 nucleotides, greater than or equal to 12,000 nucleotides, greater than or equal to 13,000 nucleotides, greater than or equal to 14,000 nucleotides, greater than or equal to 15,000 nucleotides, greater than or equal to 16,000 nucleotides, greater than or equal to 17,000 nucleotides, or greater than or equal to 18,000 nucleotides. In certain embodiments, the nucleic acid (e.g., RNA, such as mRNA) comprises less than or equal to 20,000 nucleotides, less than or equal to 15,000 nucleotides, less than or equal to 14,000 nucleotides, less than or equal to 13,000 nucleotides, less than or equal to 12,000 nucleotides, less than or equal to 11,000 nucleotides, 10,000 nucleotides, less than or equal to 9,000 nucleotides, less than or equal to 8,000 nucleotides, less than or equal to 7,000 nucleotides, or less than or equal to 6,000 nucleotides. Combinations of these ranges are also possible (e.g., greater than or equal to 400 nucleotides and less than or equal to 20,000 nucleotides, greater than or equal to 400 nucleotides and less than or equal to 15,000 nucleotides, or greater than or equal to 4,000 nucleotides and less than or equal to 6,000 nucleotides).

Without wishing to be bound by theory, it is believed that it is more difficult to achieve sufficient stability in nucleic acids (e.g., RNA, such as mRNA) the more nucleotides it has. For example, in some cases, a trans-esterification reaction at a nucleotide of an mRNA can cleave the mRNA, such that it no longer encodes the desired protein. The more nucleotides there are in an mRNA strand, the higher the statistical likelihood that one of the nucleotides will be cleaved.

In some embodiments, the article and/or liquid pharmaceutical composition comprises a lipid carrier. Examples of lipid carriers include lipid nanoparticles, liposomes, and/or lipoplex. In certain embodiments, the nucleic acid (e.g., RNA, such as mRNA) is encapsulated within the lipid carrier (e.g., lipid nanoparticle, liposome, and/or lipoplex).

### Lipid Formulations

In some embodiments, the nucleic acids of are formulated as a lipid composition, such as a composition comprising a lipid nanoparticle, a liposome, and/or a lipoplex. In some embodiments, nucleic acids of the invention are formulated as lipid nanoparticle (LNP) compositions. Lipid nanoparticles typically comprise amino lipid, non-cationic lipid, structural lipid, and PEG lipid components along with the nucleic acid cargo of interest. The lipid nanoparticles of the invention can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2017/038426; PCT/US2014/027077; PCT/US2014/055394; PCT/US2016/52117; PCT/US2012/069610; PCT/US2017/027492; PCT/US2016/059575; PCT/US2016/069491; PCT/US2016/069493; and PCT/US2014/66242, all of which are incorporated by reference herein in their entirety.

In some embodiments, the lipid nanoparticle comprises at least one ionizable amino lipid, at least one non-cationic lipid, at least one sterol, and/or at least one polyethylene glycol (PEG)-modified lipid.

In some embodiments, the lipid nanoparticle comprises a molar ratio of 20-60% ionizable amino lipid, 5-25% non-cationic lipid, 25-55% structural lipid, and 0.5-15% PEG-modified lipid.

In some embodiments, the lipid nanoparticle comprises a molar ratio of 20-60% ionizable amino lipid, 5-30% non-cationic lipid, 10-55% structural lipid, and 0.5-15% PEG-modified lipid.

In some embodiments, the lipid nanoparticle comprises 40-50 mol % ionizable lipid, optionally 45-50 mol %, for example, 45-46 mol %, 46-47 mol %, 47-48 mol %, 48-49 mol %, or 49-50 mol % for example about 45 mol %, 45.5 mol %, 46 mol %, 46.5 mol %, 47 mol %, 47.5 mol %, 48 mol %, 48.5 mol %, 49 mol %, or 49.5 mol %.

In some embodiments, the lipid nanoparticle comprises 20-60 mol % ionizable amino lipid. For example, the lipid nanoparticle may comprise 20-50 mol %, 20-40 mol %, 20-30 mol %, 30-60 mol %, 30-50 mol %, 30-40 mol %, 40-60 mol %, 40-50 mol %, or 50-60 mol % ionizable amino lipid. In some embodiments, the lipid nanoparticle comprises 20 mol %, 30 mol %, 40 mol %, 50 mol %, or 60 mol % ionizable amino lipid. In some embodiments, the lipid nanoparticle comprises 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, 50 mol %, 51 mol %, 52 mol %, 53 mol %, 54 mol %, or 55 mol % ionizable amino lipid.

In some embodiments, the lipid nanoparticle comprises 45-55 mole percent (mol %) ionizable amino lipid. For example, lipid nanoparticle may comprise 45, 46, 47, 48, 49, 50, 51, 52, 53, 54, or 55 mol % ionizable amino lipid.

Ionizable Amino Lipids

In some embodiments, the ionizable amino lipid of the present disclosure is a compound of Formula (AI):

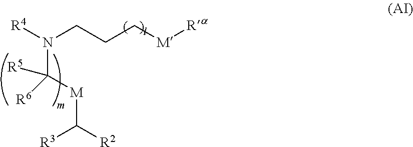

or its N-oxide, or a salt or isomer thereof,

wherein R′^{a} is R′^{branched}; wherein

R′^{branched} is:

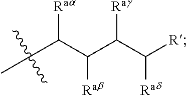

wherein

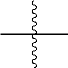

denotes a point of attachment;

wherein R^{aα}, R^{aβ}, R^{aγ}, and R^{aδ} are each independently selected from the group consisting of H, C₂₋₁₂ alkyl, and C₂₋₁₂ alkenyl;

R² and R³ are each independently selected from the group consisting of C₁₋₁₄ alkyl and C₂₋₁₄ alkenyl;

R⁴ is selected from the group consisting of —(CH₂)ₙOH, wherein n is selected from the group consisting of 1, 2, 3, 4, and 5, and

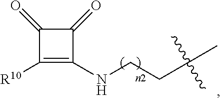

wherein

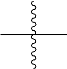

denotes a point of attachment; wherein

R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;

each R⁵ is independently selected from the group consisting of C₁₋₃ alkyl, C₂₋₃ alkenyl, and H;

each R⁶ is independently selected from the group consisting of C₁₋₃ alkyl, C₂₋₃ alkenyl, and H;

M and M′ are each independently selected from the group consisting of —C(O)O— and —OC(O)—;

R′ is a C₁₋₁₂ alkyl or C₂₋₁₂ alkenyl;

l is selected from the group consisting of 1, 2, 3, 4, and 5; and

m is selected from the group consisting of 5, 6, 7, 8, 9, 10, 11, 12, and 13.

In some embodiments of the compounds of Formula (AI), R′^{a} is R′^{branched}; R′^{branched} is

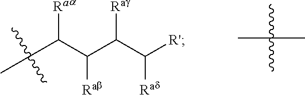

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments of the compounds of Formula (AI), R′^{a} is R′^{branched}; R′^{branched} is

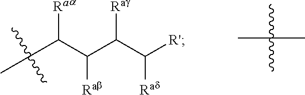

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 3; and m is 7.

In some embodiments of the compounds of Formula (AI), R′^{a} is R′^{branched}; R′^{branched} is

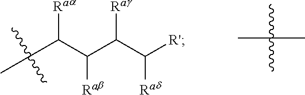

denotes a point of attachment; R^{aα} is C₂₋₁₂ alkyl; R^{aβ}, R^{aγ}, and R^{aδ} are each H; R² and R³ are each C₁₋₁₄ alkyl; R⁴ is H

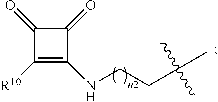

R¹⁰ NH(C₁₋₆ alkyl); n2 is 2; R⁵ is H; each R⁶ is H; M and M′ are each —C(O)O—; R′ is a C₁₋₁₂ alkyl; 1 is 5; and m is 7.

In some embodiments of the compounds of Formula (I), R′^{a} is R′^{branched}; R′^{branched} is

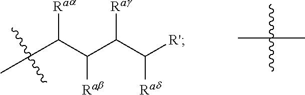

denotes a point of attachment; R^{aα}, R^{aβ}, and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments, the compound of Formula (I) is selected from:

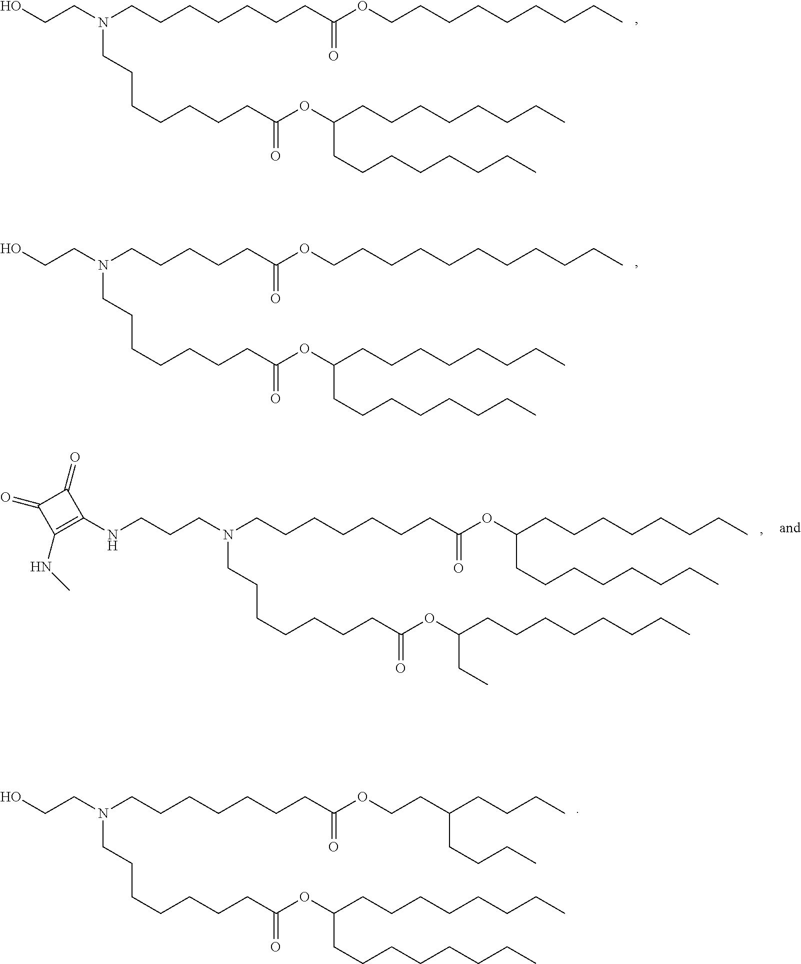 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC ; CCCCCCCCC(CC)OC(=O)CCCCCCCN(CCCCCCCC(=O)OC(CCCCCCCC)CCCCCCCC)CCCNc1c(NC)c(=O)c1=O ; CCCCCCCCC(CCCCCCCC)OC(=O)CCCCCCCN(CCO)CCCCCCCC(=O)OCCC(CCCC)CCCC]

In some embodiments, the ionizable amino lipid is a compound of Formula (AIa):

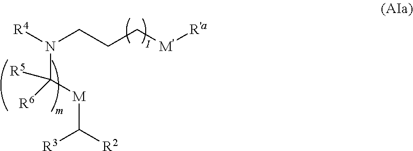

or its N-oxide, or a salt or isomer thereof,
wherein R′^{a} is R′^{branched}; wherein

R′^{branched} is:

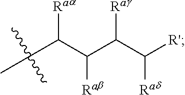

wherein

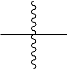

denotes a point of attachment;

wherein R^{aβ}, R^{aγ}, and R^{aδ} are each independently selected from the group consisting of H, C₂₋₁₂ alkyl, and C₂₋₁₂ alkenyl;

R² and R³ are each independently selected from the group consisting of C₁₋₁₄ alkyl and C₂₋₁₄ alkenyl;

R⁴ is selected from the group consisting of —(CH₂)ₙOH wherein n is selected from the group consisting of 1, 2, 3, 4, and 5, and

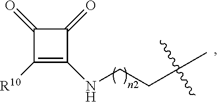

wherein

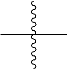

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;

each R⁵ is independently selected from the group consisting of C₁₋₃ alkyl, C₂₋₃ alkenyl, and H;

each R⁶ is independently selected from the group consisting of C₁₋₃ alkyl, C₂₋₃ alkenyl, and H;

M and M′ are each independently selected from the group consisting of —C(O)O— and —OC(O)—;

R′ is a C₁₋₁₂ alkyl or C₂₋₁₂ alkenyl;

l is selected from the group consisting of 1, 2, 3, 4, and 5; and

m is selected from the group consisting of 5, 6, 7, 8, 9, 10, 11, 12, and 13.

In some embodiments, the ionizable amino lipid is a compound of Formula (AIb):

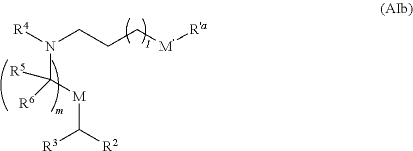

or its N-oxide, or a salt or isomer thereof,

wherein R′^{a} is R′^{branched}; wherein

R′^{branched} is:

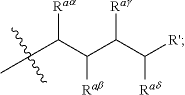

wherein

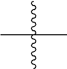

denotes a point of attachment;

wherein R^{aα}, R^{aβ}, R^{aγ}, and R^{aδ} are each independently selected from the group consisting of H, C₂₋₁₂ alkyl, and C₂₋₁₂ alkenyl;

R² and R³ are each independently selected from the group consisting of C₁₋₁₄ alkyl and C₂₋₁₄ alkenyl;

R⁴ is —(CH₂)ₙOH, wherein n is selected from the group consisting of 1, 2, 3, 4, and 5;

each R⁵ is independently selected from the group consisting of C₁₋₃ alkyl, C₂₋₃ alkenyl, and H;

each R⁶ is independently selected from the group consisting of C₁₋₃ alkyl, C₂₋₃ alkenyl, and H;

M and M′ are each independently selected from the group consisting of —C(O)O— and —OC(O)—;

R′ is a C₁₋₁₂ alkyl or C₂₋₁₂ alkenyl;

l is selected from the group consisting of 1, 2, 3, 4, and 5; and

m is selected from the group consisting of 5, 6, 7, 8, 9, 10, 11, 12, and 13.

In some embodiments of Formula (AI) or (AIb), R′^{a} is R′^{branched}; R′^{branched} is

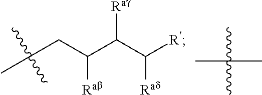

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments of Formula (AI) or (AIb), R′^{a} is R′^{branched}; R′^{branched} is

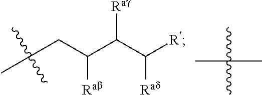

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 3; and m is 7.

In some embodiments of Formula (AI) or (AIb), R′^{a} is R′^{branched}; R′^{branched} is

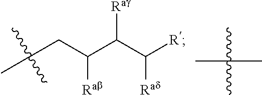

denotes a point of attachment; R^{aβ} and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments, the ionizable amino lipid is a compound of Formula (AIc):

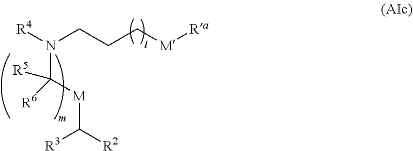

or its N-oxide, or a salt or isomer thereof,

wherein R′^{a} is R′^{branched}; wherein

R′^{branched} is:

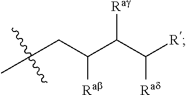

wherein

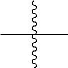

denotes a point of attachment;

wherein R^{aα}, R^{aβ}, R^{aγ}, and R^{aδ} are each independently selected from the group consisting of H, C₂₋₁₂ alkyl, and C₂₋₁₂ alkenyl;

R² and R³ are each independently selected from the group consisting of C₁₋₁₄ alkyl and C₂₋₁₄ alkenyl;

R⁴ is

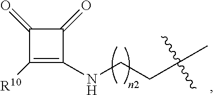

wherein

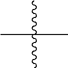

denotes a point of attachment; wherein R¹⁰ is N(R)₂; each R is independently selected from the group consisting of C₁₋₆ alkyl, C₂₋₃ alkenyl, and H; n2 is selected from the group consisting of 1, 2, 3, 4, 5, 6, 7, 8, 9, and 10;

each R⁵ is independently selected from the group consisting of C₁₋₃ alkyl, C₂₋₃ alkenyl, and H;

each R⁶ is independently selected from the group consisting of C₁₋₃ alkyl, C₂₋₃ alkenyl, and H;

M and M′ are each independently selected from the group consisting of —C(O)O— and —OC(O)—;

R′ is a C₁₋₁₂ alkyl or C₂₋₁₂ alkenyl;

l is selected from the group consisting of 1, 2, 3, 4, and 5; and

m is selected from the group consisting of 5, 6, 7, 8, 9, 10, 11, 12, and 13.

In some embodiments, R′^{a} is R′^{branched}; R′^{branched} is

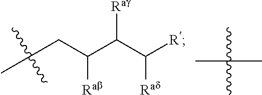

denotes a point of attachment; R^{aβ}, R^{aγ}, and R^{aδ} are each H; R^{aα} is C₂₋₁₂ alkyl; R² and R³ are each C₁₋₁₄ alkyl; R⁴ is

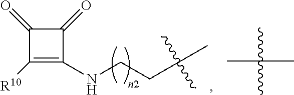

denotes a point of attachment; R¹⁰ is NH(C₁₋₆ alkyl); n2 is 2; each R⁵ is H; each R⁶ is H; M and M′ are each —C(O)O—; R′ is a C₁₋₁₂ alkyl; 1 is 5; and m is 7.

In some embodiments, the compound of Formula (AIc) is:

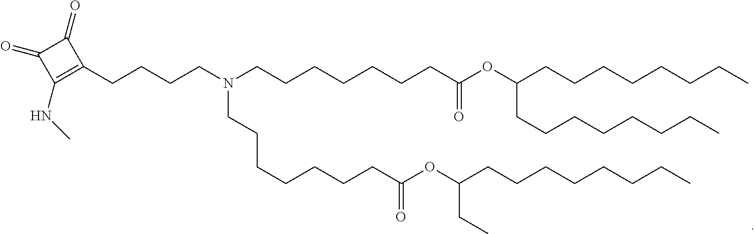 [SMILES: CCCCCCCCC(CC)OC(=O)CCCCCCCN(CCCCCCCC(=O)OC(CCCCCCCC)CCCCCCCC)CCCCc1c(NC)c(=O)c1=O]

In some embodiments, the ionizable amino lipid is a compound of Formula (AII):

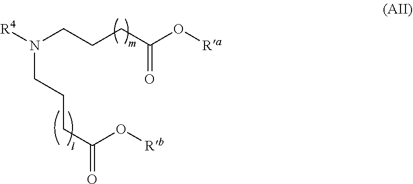

or its N-oxide, or a salt or isomer thereof,

wherein R′^{a} is R′^{branched} or R′^{cyclic}; wherein

R′^{branched} is

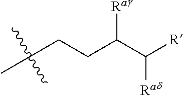

### and R′^{cyclic} is:

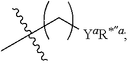

and

R′^{b} is:

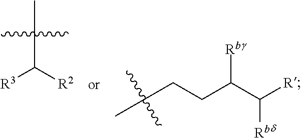

wherein

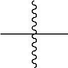

denotes a point of attachment;

R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;

R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;

R² and R³ are each independently selected from the group consisting of C₁₋₁₄ alkyl and C₂₋₁₄ alkenyl;

R⁴ is selected from the group consisting of —(CH₂)ₙOH wherein n is selected from the group consisting of 1, 2, 3, 4, and 5, and

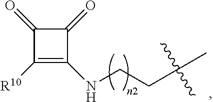

wherein

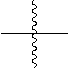

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;

each R′ independently is a C₁₋₁₂ alkyl or C₂₋₁₂ alkenyl;

Y^{a} is a C₃₋₆ carbocycle;

R*″^{a} is selected from the group consisting of C₁₋₁₅ alkyl and C₂₋₁₅ alkenyl; and

s is 2 or 3;

m is selected from 1, 2, 3, 4, 5, 6, 7, 8, and 9;

l is selected from 1, 2, 3, 4, 5, 6, 7, 8, and 9.

In some embodiments, the ionizable amino lipid is a compound of Formula (AII-a):

or its N-oxide, or a salt or isomer thereof,

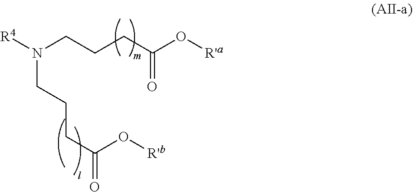

wherein R′^{a} is R′^{branched} or R′^{cyclic}; wherein

R′^{branched} is:

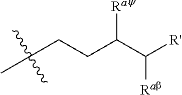

### and R′^{b} is:

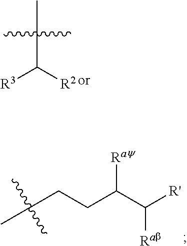

wherein

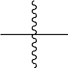

denotes a point of attachment;

R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;

R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;

R² and R³ are each independently selected from the group consisting of C₁₋₁₄ alkyl and C₂₋₁₄ alkenyl;

R⁴ is selected from the group consisting of —(CH₂)ₙOH wherein n is selected from the group consisting of 1, 2, 3, 4, and 5, and

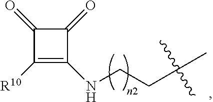

wherein

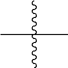

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;

each R′ independently is a C₁₋₁₂ alkyl or C₂₋₁₂ alkenyl;

m is selected from 1, 2, 3, 4, 5, 6, 7, 8, and 9;

l is selected from 1, 2, 3, 4, 5, 6, 7, 8, and 9.

In some embodiments, the ionizable amino lipid is a compound of Formula (AII-b):

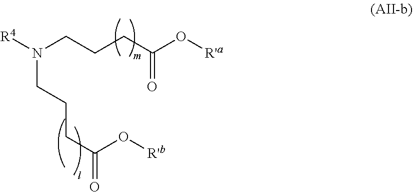

or its N-oxide, or a salt or isomer thereof,

wherein R′^{a} is R′^{branched} or R′^{cyclic}; wherein

R′^{branched} is:

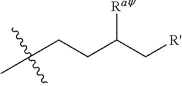

### and R′^{b} is:

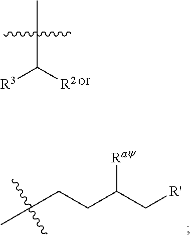

wherein

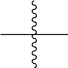

denotes a point of attachment;

R^{aγ} and R^{bγ} are each independently selected from the group consisting of C₁₋₁₂ alkyl and C₂₋₁₂ alkenyl;

R² and R³ are each independently selected from the group consisting of C₁₋₁₄ alkyl and C₂₋₁₄ alkenyl;

R⁴ is selected from the group consisting of —(CH₂)ₙOH wherein n is selected from the group consisting of 1, 2, 3, 4, and 5, and

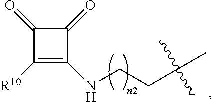

wherein

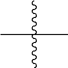

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;

each R′ independently is a C₁₋₁₂ alkyl or C₂₋₁₂ alkenyl;

m is selected from 1, 2, 3, 4, 5, 6, 7, 8, and 9;

l is selected from 1, 2, 3, 4, 5, 6, 7, 8, and 9.

In some embodiments, the ionizable amino lipid is a compound of Formula (AII-c):

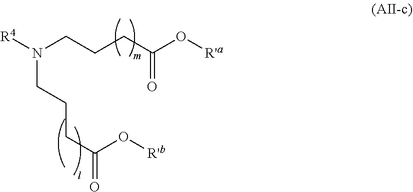

or its N-oxide, or a salt or isomer thereof,

wherein R′^{a} is R′^{branched} or R′^{cyclic}; wherein

R′^{branched} is:

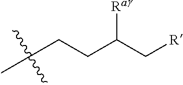

### and R′^{b} is:

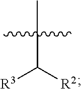

wherein

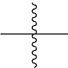

denotes a point of attachment;

wherein R^{aγ} is selected from the group consisting of C₁₋₁₂ alkyl and C₂₋₁₂ alkenyl;

R² and R³ are each independently selected from the group consisting of C₁₋₁₄ alkyl and C₂₋₁₄ alkenyl;

R⁴ is selected from the group consisting of —(CH₂)ₙOH wherein n is selected from the group consisting of 1, 2, 3, 4, and 5, and

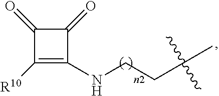

wherein

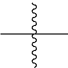

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;

R′ is a C₁₋₁₂ alkyl or C₂₋₁₂ alkenyl;

m is selected from 1, 2, 3, 4, 5, 6, 7, 8, and 9;

l is selected from 1, 2, 3, 4, 5, 6, 7, 8, and 9.

In some embodiments, the ionizable amino lipid is a compound of Formula (AII-d):

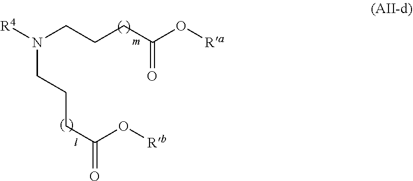

or its N-oxide, or a salt or isomer thereof,

wherein R′^{a} is R′^{branched} or R′ cyclic; wherein

R′^{branched} is:

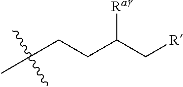

### and R′^{b} is:

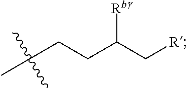

wherein

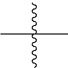

denotes a point of attachment;

wherein R^{aγ} and R^{bγ} are each independently selected from the group consisting of C₁₋₁₂ alkyl and C₂₋₁₂ alkenyl;

R⁴ is selected from the group consisting of —(CH₂)ₙOH wherein n is selected from the group consisting of 1, 2, 3, 4, and 5, and

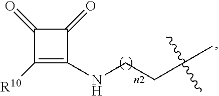

wherein

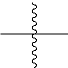

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;

each R′ independently is a C₁₋₁₂ alkyl or C₂₋₁₂ alkenyl;

m is selected from 1, 2, 3, 4, 5, 6, 7, 8, and 9;

l is selected from 1, 2, 3, 4, 5, 6, 7, 8, and 9.

In some embodiments, the ionizable amino lipid is a compound of Formula (AII-e):

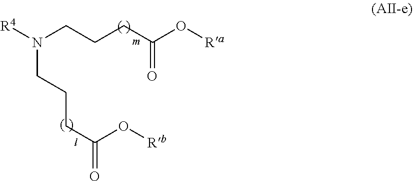

or its N-oxide, or a salt or isomer thereof,

wherein R′^{a} is R′^{branched} or R′^{cyclic}; wherein

R′^{branched} is:

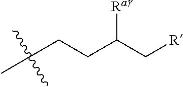

### and R′^{b} is:

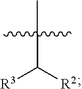

wherein

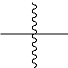

denotes a point of attachment;

wherein R^{aγ} is selected from the group consisting of C₁₋₁₂ alkyl and C₂₋₁₂ alkenyl;

R² and R³ are each independently selected from the group consisting of C₁₋₁₄ alkyl and C₂₋₁₄ alkenyl;

R⁴ is —(CH₂)ₙOH wherein n is selected from the group consisting of 1, 2, 3, 4, and 5;

R′ is a C₁₋₂ alkyl or C₂₋₁₂ alkenyl;

m is selected from 1, 2, 3, 4, 5, 6, 7, 8, and 9;

l is selected from 1, 2, 3, 4, 5, 6, 7, 8, and 9.

In some embodiments of the compound of Formula (AII), (AII-a), (AII-b), (AII-c), (AII-d), or (AII-e), m and l are each independently selected from 4, 5, and 6. In some embodiments of the compound of Formula (AII), (AII-a), (AII-b), (AII-c), (AII-d), or (AII-e), m and l are each 5.

In some embodiments of the compound of Formula (AII), (AII-a), (AII-b), (AII-c), (AII-d), or (AII-e), each R′ independently is a C₁₋₁₂ alkyl. In some embodiments of the compound of Formula (AII), (AII-a), (AII-b), (AII-c), (AII-d), or (AII-e), each R′ independently is a C₂₋₅ alkyl.

In some embodiments of the compound of Formula (AII), (AII-a), (AII-b), (AII-c), (AII-d), or (AII-e), R′^{b} is:

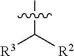

and R² and R³ are each independently a C₁₋₁₄ alkyl. In some embodiments of the compound of Formula (AII), (AII-a), (AII-b), (AII-c), (AII-d), or (AII-e), R′^{b} is:

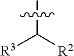

and R² and R³ are each independently a C₆₋₁₀ alkyl. In some embodiments of the compound of Formula (AII), (AII-a), (AII-b), (AII-c), (AII-d), or (AII-e), R′^{b} is:

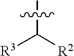

and R² and R³ are each a C₈ alkyl.

In some embodiments of the compound of Formula (AII), (AII-a), (AII-b), (AII-c), (AII-d), or (AII-e), R′^{branched} is:

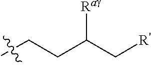

### and R′^{b} is:

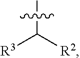

R^{aγ} is a C₁₋₁₂ alkyl and R² and R³ are each independently a C₆₋₁₀ alkyl. In some embodiments of the compound of Formula (AII), (AII-a), (AII-b), (AII-c), (AII-d), or (AII-e), R′^{branched} is:

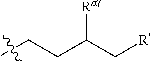

### and R′^{b} is:

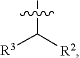

R^{aγ} is a C₂₋₆ alkyl and R² and R³ are each independently a C₆₋₁₀ alkyl. In some embodiments of the compound of Formula (AII), (AII-a), (AII-b), (AII-c), (AII-d), or (AII-e), R′^{branched} is:

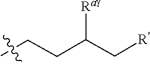

### and R′^{b} is:

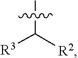

R^{aγ} is a C₂₋₆ alkyl, and R² and R³ are each a C₈ alkyl.

In some embodiments of the compound of Formula (AII), (AII-a), (AII-b), (AII-c), (AII-d), or (AII-e), R′^{branched} is:

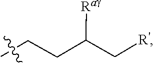

### R′^{b} is:

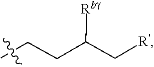

and R^{aγ} and R^{bγ} are each a C₁₋₁₂ alkyl. In some embodiments of the compound of Formula (AII), (AII-a), (AII-b), (AII-c), (AII-d), or (AII-e), R′^{branched} is:

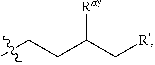

### R′^{b} is:

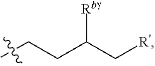

and R^{aγ} and R^{bγ} are each a C₂₋₆ alkyl.

In some embodiments of the compound of Formula (AII), (AII-a), (AII-b), (AII-c), (AII-d), or (AII-e), m and l are each independently selected from 4, 5, and 6 and each R′ independently is a C₁₋₁₂ alkyl. In some embodiments of the compound of Formula (AII), (AII-a), (AII-b), (AII-c), (AII-d), or (AII-e), m and l are each 5 and each R′ independently is a C₂₋₅ alkyl.

In some embodiments of the compound of (AII), (AII-a), (AII-b), (AII-c), (AII-d), or (AII-e), R′^{branched} is:

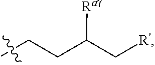

### R′^{b} is:

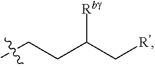

m and l are each independently selected from 4, 5, and 6, each R′ independently is a C₁₋₁₂ alkyl, and R^{aγ} and R^{bγ} are each a C₁₋₁₂ alkyl. In some embodiments of the compound of Formula (AII), (AII-a), (AII-b), (AII-c), (AII-d), or (AII-e), R′^{branched} is:

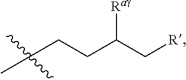

### R′^{b} is:

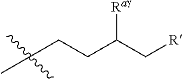

m and l are each 5, each R′ independently is a C₂₋₅ alkyl, and R^{aγ} and R^{bγ} are each a C₂₋₆ alkyl.

In some embodiments of the compound of Formula (AII), (AII-a), (AII-b), (AII-c), (AII-d), or (AII-e), R′^{branched} is:

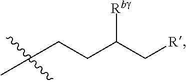

### and R′^{b} is:

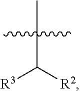

m and l are each independently selected from 4, 5, and 6, R′ is a C₁₋₁₂ alkyl, R^{aγ} is a C₁₋₁₂ alkyl and R² and R³ are each independently a C₆₋₁₀ alkyl.

In some embodiments of the compound of Formula (AII), (AII-a), (AII-b), (AII-c), (AII-d), or (AII-e), R′^{branched} is:

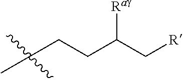

### and R′^{b} is:

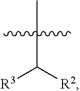

m and l are each 5, R′ is a C₂₋₅ alkyl, R^{aγ} is a C₂₋₆ alkyl, and R² and R³ are each a C₈ alkyl.

In some embodiments of the compound of (AII), (AII-a), (AII-b), (AII-c), (AII-d), or (AII-e), R⁴ is

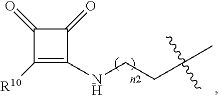

wherein R¹⁰ is NH(C₁₋₆ alkyl) and n2 is 2. In some embodiments of the compound of Formula (AII), (AII-a), (AII-b), (AII-c), (AII-d), or (AII-e), R⁴ is

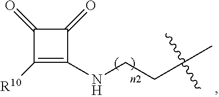

wherein R¹⁰ is NH(CH₃) and n2 is 2.

In some embodiments of the compound of Formula (AII), (AII-a), (AII-b), (AII-c), (AII-d), or (AII-e), R′^{branched} is:

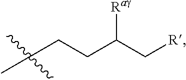

### R′^{b} is:

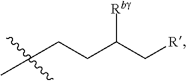

m and l are each independently selected from 4, 5, and 6, each R′ independently is a C₁₋₁₂ alkyl, R^{aγ} and R^{bγ} are each a C₁₋₁₂ alkyl, and R⁴ is

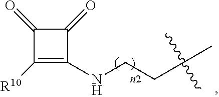

wherein R¹⁰ is NH(C₁₋₆ alkyl), and n2 is 2. In some embodiments of the compound of Formula (AII), (AII-a), (AII-b), (AII-c), (AII-d), or (AII-e), R′^{branched} is:

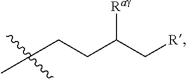

### R′^{b} is:

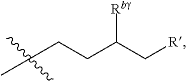

m and l are each 5, each R′ independently is a C₂₋₅ alkyl, R^{aγ} and R^{bγ} are each a C₂₋₆ alkyl, and R⁴ is

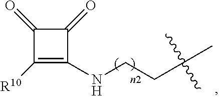

wherein R¹⁰ is NH(CH₃) and n2 is 2.

In some embodiments of the compound of Formula (AII), (AII-a), (AII-b), (AII-c), (AII-d), or (AII-e), R′^{branched} is:

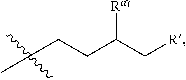

### and R′^{b} is:

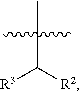

m and l are each independently selected from 4, 5, and 6, R′ is a C₁₋₁₂ alkyl, R² and R³ are each independently a C₆₋₁₀ alkyl, R^{aγ} is a C₁₋₁₂ alkyl, and R⁴ is

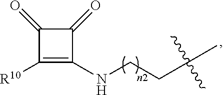

wherein R¹⁰ is NH(C₁₋₆ alkyl) and n2 is 2. In some embodiments of the compound of Formula (AII), (AII-a), (AII-b), (AII-c), (AII-d), or (AII-e), R′^{branched} is:

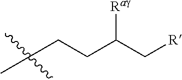

### and R′^{b} is:

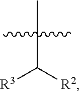

m and l are each 5, R′ is a C₂₋₅ alkyl, R^{aγ} is a C₂₋₆ alkyl, R² and R³ are each a C₈ alkyl, and R⁴ is

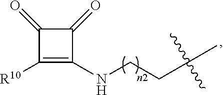

wherein R¹⁰ is NH(CH₃) and n2 is 2.

In some embodiments of the compound of Formula (AII), (AII-a), (AII-b), (AII-c), (AII-d), or (AII-e), R⁴ is —(CH₂)ₙOH and n is 2, 3, or 4. In some embodiments of the compound of Formula (AII), (AII-a), (AII-b), (AII-c), (AII-d), or (AII-e), R⁴ is —(CH₂)ₙOH and n is 2.

In some embodiments of the compound of Formula (AII), (AII-a), (AII-b), (AII-c), (AII-d), or (AII-e), R′^{branched} is:

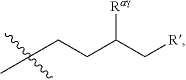

### R′^{b} is:

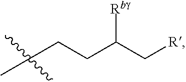

m and l are each independently selected from 4, 5, and 6, each R′ independently is a C₁₋₁₂ alkyl, R^{aγ} and R^{bγ} are each a C₁₋₁₂ alkyl, R⁴ is —(CH₂)ₙOH, and n is 2, 3, or 4. In some embodiments of the compound of Formula (AII), (AII-a), (AII-b), (AII-c), (AII-d), or (AII-e), R′^{branched} is:

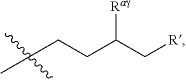

### R′^{b} is:

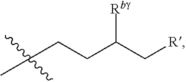

m and l are each 5, each R′ independently is a C₂₋₅ alkyl, R^{aγ} and R^{bγ} are each a C₂₋₆ alkyl, R⁴ is —(CH₂)ₙOH, and n is 2.

In some embodiments, the ionizable amino lipid is a compound of Formula (AII-f):

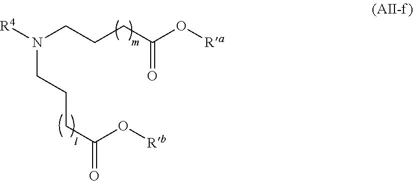

or its N-oxide, or a salt or isomer thereof,

wherein R′^{a} is R′^{branched} or R′^{cyclic}; wherein

R′^{branched} is:

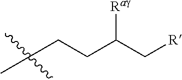

### and R′^{b} is:

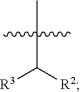

wherein

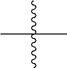

denotes a point of attachment;

R^{aγ} is a C₁₋₁₂ alkyl;

R² and R³ are each independently a C₁₋₁₄ alkyl;

R⁴ is —(CH₂)ₙOH wherein n is selected from the group consisting of 1, 2, 3, 4, and 5;

R′ is a C₁₋₁₂ alkyl;

m is selected from 4, 5, and 6; and

l is selected from 4, 5, and 6.

In some embodiments of the compound of Formula (AII-f), m and l are each 5, and n is 2, 3, or 4.

In some embodiments of the compound of Formula (AII-f) R′ is a C₂₋₅ alkyl, R^{a} is a C₂₋₆ alkyl, and R² and R³ are each a C₆₋₁₀ alkyl.

In some embodiments of the compound of Formula (AII-f), m and l are each 5, n is 2, 3, or 4, R′ is a C₂₋₅ alkyl, R^{a} is a C₂₋₆ alkyl, and R² and R³ are each a C₆₋₁₀ alkyl.

In some embodiments, the ionizable amino lipid is a compound of Formula (AII-g):

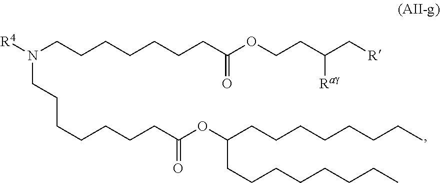

wherein

R^{aγ} is a C₂₋₆ alkyl;

R′ is a C₂₋₅ alkyl; and

R⁴ is selected from the group consisting of —(CH₂)ₙOH wherein n is selected from the group consisting of 3, 4, and 5, and

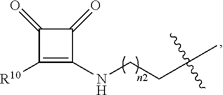

wherein

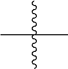

denotes a point of attachment, R¹⁰ is NH(C₁₋₆ alkyl), and n2 is selected from the group consisting of 1, 2, and 3.

In some embodiments, the ionizable amino lipid is a compound of Formula (AII-h):

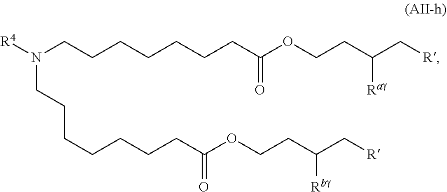

wherein

R^{aγ} and R^{bγ} are each independently a C₂₋₆ alkyl;

each R′ independently is a C₂₋₅ alkyl; and

R⁴ is selected from the group consisting of —(CH₂)ₙOH wherein n is selected from the group consisting of 3, 4, and 5, and

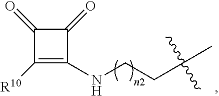

wherein

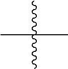

denotes a point of attachment, R¹⁰ is NH(C₁₋₆ alkyl), and n2 is selected from the group consisting of 1, 2, and 3.

In some embodiments of the compound of Formula (AII-g) or (AII-h), R⁴ is

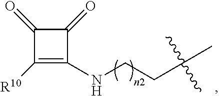

wherein

R¹⁰ is NH(CH₃) and n2 is 2.

In some embodiments of the compound of Formula (AII-g) or (AII-h), R⁴ is —(CH₂)₂OH.

In some embodiments, the ionizable amino lipids of the present disclosure may be one or more of compounds of Formula (VI):

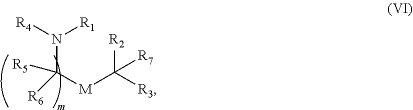

or their N-oxides, or salts or isomers thereof, wherein:

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of hydrogen, a C₃₋₆ carbocycle, —(CH₂)ₙQ, —(CH₂)ₙCHQR,

—CHQR, —CQ(R)₂, and unsubstituted C₁₋₆ alkyl, where Q is selected from a carbocycle, heterocycle, —OR, —O(CH₂)n N(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —N(R)S(O)₂R⁸, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —OC(O)-M″—C(O)O—, —C(O)N(R′)—,

—N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;

R₇ is selected from the group consisting of C₁₋₃ alkyl, C₂₋₃ alkenyl, and H;

R₈ is selected from the group consisting of C₃₋₆ carbocycle and heterocycle;

R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₅ alkyl and C₃₋₁₅ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13; and wherein when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In some embodiments, another subset of compounds of Formula (VI) includes those in which: R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and a 5- to 14-membered heterocycloalkyl having one or more heteroatoms selected from N, O, and S which is substituted with one or more substituents selected from oxo (═O), OH, amino, mono- or di-alkylamino, and C₁₋₃ alkyl, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

R⁸ is selected from the group consisting of C₃₋₆ carbocycle and heterocycle;

R₉ is selected from the group consisting of H, CN, N02, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13, or salts or isomers thereof.

In some embodiments, another subset of compounds of Formula (VI) includes those in which:

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5 to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N (R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is —CHQR, and —CQ(R)₂, then Q is either a 5- to 14-membered heteroaryl or 8- to 14-membered heterocycloalkyl;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

R⁸ is selected from the group consisting of C₃₋₆ carbocycle and heterocycle;

R₉ is selected from the group consisting of H, CN, N02, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13, or salts or isomers thereof.

In some embodiments, another subset of compounds of Formula (VI) includes those in which:

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

R⁸ is selected from the group consisting of C₃₋₆ carbocycle and heterocycle;

R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or isomers thereof.

In some embodiments, another subset of compounds of Formula (VI) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is —(CH₂)ₙQ or —(CH₂)ₙCHQR, where Q is —N(R)₂, and n is selected from 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₁₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13, or salts or isomers thereof.

In some embodiments, another subset of compounds of Formula (VI) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of —(CH₂)ₙQ, —(CH₂)ₙCHQR, —CHQR, and —CQ(R)₂, where Q is —N(R)₂, and n is selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₁₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13, or salts or isomers thereof.

In certain embodiments, a subset of compounds of Formula (VI) includes those of Formula (VI-A):

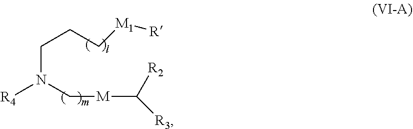

or its N-oxide, or a salt or isomer thereof, wherein l is selected from 1, 2, 3, 4, and 5; m is selected from 5, 6, 7, 8, and 9; M₁ is a bond or M′; R₄ is hydrogen, unsubstituted C₁₋₃ alkyl, or —(CH₂)ₙQ, in which Q is

OH, —NHC(S)N(R)₂, —NHC(O)N(R)₂, —N(R)C(O)R, —N(R)S(O)₂R, —N(R)R₈,
—NHC(═NR₉)N(R)₂, —NHC(═CHR₉)N(R)₂, —OC(O)N(R)₂, —N(R)C(O)OR, heteroaryl or heterocycloalkyl; M and M′ are independently selected
from —C(O)O—, —OC(O)—, —OC(O)-M″—C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (VI) includes those of Formula (VI-B):

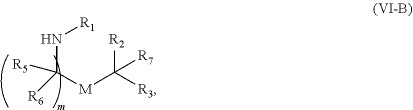

or its N-oxide, or a salt or isomer thereof in which all variables are as defined herein. For example, m is selected from 5, 6, 7, 8, and 9; R₄ is hydrogen, unsubstituted C₁₋₃ alkyl, or —(CH₂)ₙQ, in which Q is
H, —NHC(S)N(R)₂, —NHC(O)N(R)₂, —N(R)C(O)R, —N(R)S(O)₂R, —N(R)R⁸,
—NHC(═NR₉)N(R)₂, —NHC(═CHR₉)N(R)₂, —OC(O)N(R)₂, —N(R)C(O)OR, heteroaryl or heterocycloalkyl; M and M′ are independently selected
from —C(O)O—, —OC(O)—, —OC(O)-M″—C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (VI) includes those of Formula (VII):

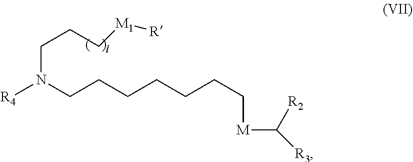

or its N-oxide, or a salt or isomer thereof, wherein l is selected from 1, 2, 3, 4, and 5; M₁ is a bond or M′; R₄ is hydrogen, unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R⁸, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″—C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, the compounds of Formula (VI) are of Formula (VIIa),

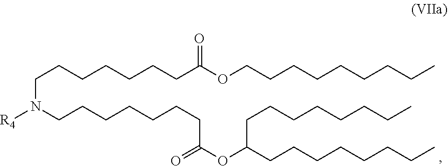

or their N-oxides, or salts or isomers thereof, wherein R₄ is as described herein.

In another embodiment, the compounds of Formula (VI) are of Formula (VIIb),

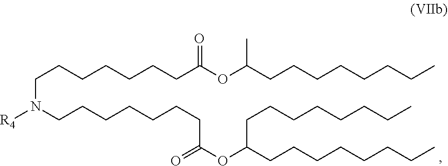

or their N-oxides, or salts or isomers thereof, wherein R₄ is as described herein.

In another embodiment, the compounds of Formula (VI) are of Formula (VIIc) or (VIIe):

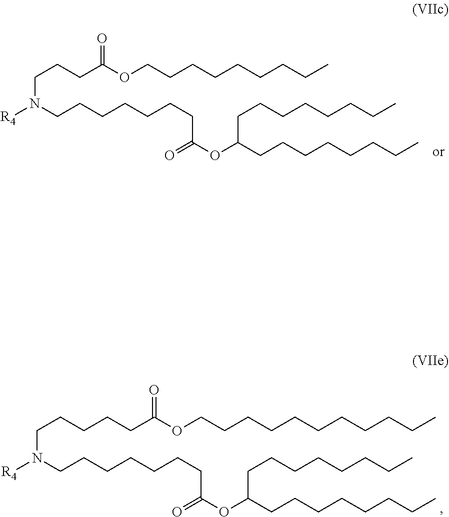

or their N-oxides, or salts or isomers thereof, wherein R₄ is as described herein.

In another embodiment, the compounds of Formula (VI) are of Formula (VIIf):

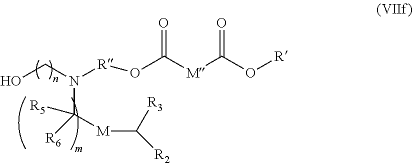

or their N-oxides, or salts or isomers thereof,

wherein M is —C(O)O— or —OC(O)—, M″ is C₁₋₆ alkyl or C₂₋₆ alkenyl, R₂ and R₃ are independently selected from the group consisting of C₅₋₁₄ alkyl and C₅₋₁₄ alkenyl, and n is selected from 2, 3, and 4.

In a further embodiment, the compounds of Formula (VI) are of Formula (VIId),

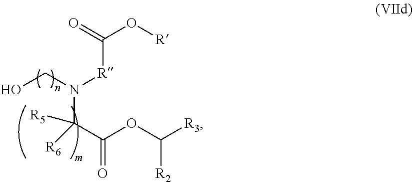

or their N-oxides, or salts or isomers thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In some embodiments, an ionizable amino lipid of the disclosure comprises a compound having structure:

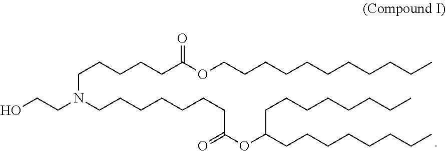 [SMILES: CCCCCCCCCCCOC(=O)CCCCCN(CCO)CCCCCCCC(=O)OC(CCCCCCCC)CCCCCCCC]

In some embodiments, an ionizable amino lipid of the disclosure comprises a compound having structure:

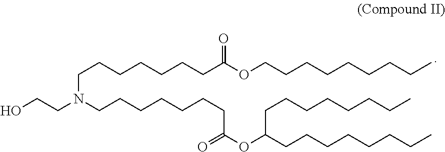 [SMILES: CCCCCCCCCOC(=O)CCCCCCCN(CCO)CCCCCCCC(=O)OC(CCCCCCCC)CCCCCCCC]

In a further embodiment, the compounds of Formula (VI) are of Formula (VIIg),

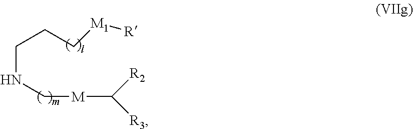

or their N-oxides, or salts or isomers thereof, wherein l is selected from 1, 2, 3, 4, and 5; m is selected from 5, 6, 7, 8, and 9; M₁ is a bond or M′; M and M′ are independently selected
from —C(O)O—, —OC(O)—, —OC(O)-M″—C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, M″ is C₁₋₆ alkyl (e.g., C₁₋₄ alkyl) or C₂₋₆ alkenyl (e.g. C₂₋₄ alkenyl). For example, R₂ and R₃ are independently selected from the group consisting of C₅₋₁₄ alkyl and C₅₋₁₄ alkenyl.

In some embodiments, the ionizable amino lipids are one or more of the compounds described in U.S. Application Nos. 62/220,091, 62/252,316, 62/253,433, 62/266,460, 62/333,557, 62/382,740, 62/393,940, 62/471,937, 62/471,949, 62/475,140, and 62/475,166, and PCT Application No. PCT/US2016/052352.

The central amine moiety of a lipid according to Formula (VI), (VI-A), (VI-B), (VII), (VIIa), (VIIb), (VIIc), (VIId), (VIIe), (VIIf), or (VIIg) may be protonated at a physiological pH. Thus, a lipid may have a positive or partial positive charge at physiological pH. Such amino lipids may be referred to as cationic lipids, ionizable lipids, cationic amino lipids, or ionizable amino lipids. Amino lipids may also be zwitterionic, i.e., neutral molecules having both a positive and a negative charge.

In some embodiments, the ionizable amino lipids of the present disclosure may be one or more of compounds of formula (VIII),

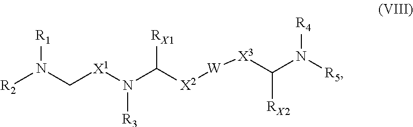

or salts or isomers thereof, wherein

W is

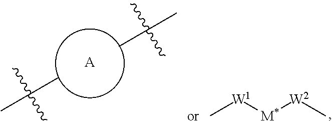

ring A is

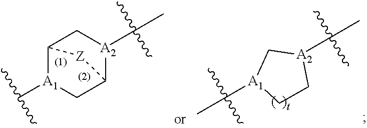

t is 1 or 2;

A₁ and A₂ are each independently selected from CH or N;

Z is CH₂ or absent wherein when Z is CH₂, the dashed lines (1) and (2) each represent a single bond; and when Z is absent, the dashed lines (1) and (2) are both absent;

R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;

R_{X1} and R_{X2} are each independently H or C₁₋₃ alkyl;

each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —C(O)S—, —SC(O)—, an aryl group, and a heteroaryl group;

M* is C₁-C₆ alkyl,

W¹ and W² are each independently selected from the group consisting of —O— and —N(R₆)—;

each R₆ is independently selected from the group consisting of H and C₁₋₅ alkyl; X¹, X², and X³ are independently selected from the group consisting of a bond, —CH₂—, —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)ₙ—OC(O)—, —C(O)O—(CH₂)ₙ—, —CH(OH)—, —C(S)—, and —CH(SH)—;

each Y is independently a C₃₋₆ carbocycle;

each R* is independently selected from the group consisting of C₁₋₁₂ alkyl and C₂₋₁₂ alkenyl;

each R is independently selected from the group consisting of C₁₋₃ alkyl and a C₃₋₆ carbocycle;

each R′ is independently selected from the group consisting of C₁₋₁₂ alkyl, C₂₋₁₂ alkenyl, and H;

each R″ is independently selected from the group consisting of C₃₋₁₂ alkyl, C₃₋₁₂ alkenyl and —R*MR′; and

n is an integer from 1-6;

wherein when ring A is

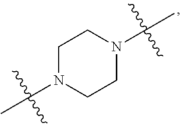 [SMILES: *N1CCN(*)CC1]

then

i) at least one of X¹, X², and X³ is not —CH₂—; and/or

ii) at least one of R₁, R₂, R₃, R₄, and R₅ is —R″MR′.

In some embodiments, the compound is of any of formulae (VIIIa1)-(VIIIa8):

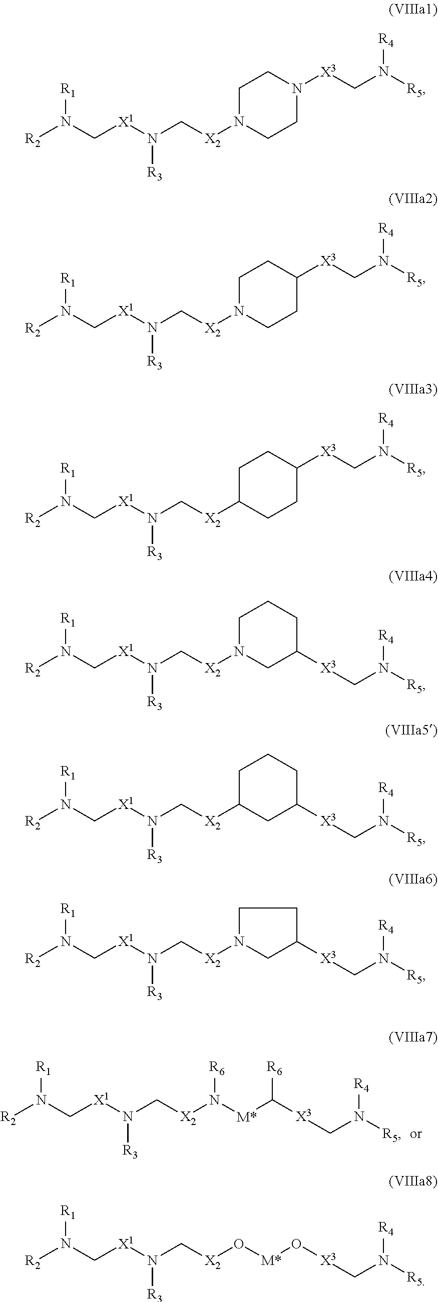

In some embodiments, the ionizable amino lipid is

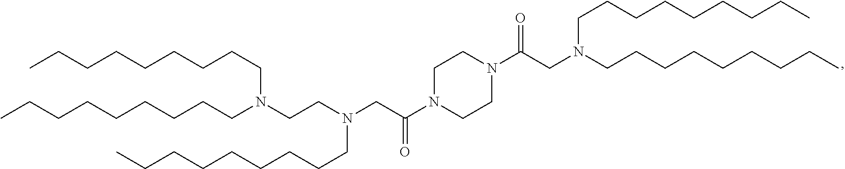 [SMILES: CCCCCCCCCN(CCCCCCCCC)CCN(CCCCCCCCC)CC(=O)N1CCN(C(=O)CN(CCCCCCCCC)CCCCCCCCC)CC1]

or a salt thereof.

The central amine moiety of a lipid according to Formula (VIII), (VIIIa1), (VIIIa2), (VIIIa3), (VIIIa4), (VIIIa5), (VIIIa6), (VIIIa7), or (VIIIa8) may be protonated at a physiological pH. Thus, a lipid may have a positive or partial positive charge at physiological pH.

In some embodiments, the lipid nanoparticle comprises a lipid having the structure:

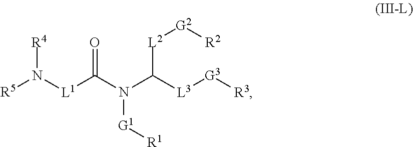

or a pharmaceutically acceptable salt, tautomer, or stereoisomer thereof, wherein:

R¹ is optionally substituted C₁-C₂₄ alkyl or optionally substituted C₂-C₂₄ alkenyl;

R² and R³ are each independently optionally substituted C₁-C₃₆ alkyl;

R⁴ and R⁵ are each independently optionally substituted C₁-C₆ alkyl, or R⁴ and R⁵ join, along with the N to which they are attached, to form a heterocyclyl or heteroaryl;

L¹, L², and L³ are each independently optionally substituted C₁—C is alkylene; G¹ is a direct bond, —(CH₂)ₙO(C═O)—, —(CH₂)ₙ(C═O)O—, or —(C═O)—; G² and G³ are each independently —(C═O)O— or —O(C═O)—; and n is an integer greater than 0.

In some embodiments, the lipid nanoparticle comprises a lipid having the structure:

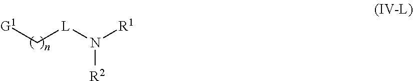

or a pharmaceutically acceptable salt, tautomer, or stereoisomer thereof, wherein:

G¹ is —N(R³)R⁴ or —OR⁵;

R¹ is optionally substituted branched, saturated or unsaturated C₁₂-C₃₆ alkyl;

R² is optionally substituted branched or unbranched, saturated or unsaturated C₁₂-C₃₆ alkyl when L is —C(═O)—; or R² is optionally substituted branched or unbranched, saturated or unsaturated C₄-C₃₆ alkyl when L is C₆-C₁₂ alkylene, C₆-C₁₂ alkenylene, or C₂-C₆ alkynylene;

R³ and R⁴ are each independently H, optionally substituted branched or unbranched, saturated or unsaturated C₁-C₆ alkyl; or R³ and R⁴ are each independently optionally substituted branched or unbranched, saturated or unsaturated C₁-C₆ alkyl when L is C₆-C₁₂ alkylene, C₆-C₁₂ alkenylene, or C₂-C₆ alkynylene; or R³ and R⁴, together with the nitrogen to which they are attached, join to form a heterocyclyl;

R⁵ is H or optionally substituted C₁-C₆ alkyl;

L is —C(═O)—, C₆-C₁₂ alkylene, C₆-C₁₂ alkenylene, or C₂-C₆ alkynylene; and

n is an integer from 1 to 12.

In some embodiments, the lipid nanoparticle comprises a lipid having the structure:

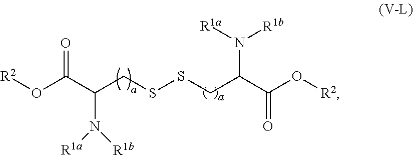

or a pharmaceutically acceptable salt thereof, wherein:

each R^{1a} is independently hydrogen, R^{1c}, or R^{1d};

each R^{1b} is independently R^{1c} or R^{1d};

each R^{1c} is independently —[CH₂]₂C(O)X¹R³;

each R^{1d} Is independently —C(O)R⁴;

each R² is independently —[C(R^{2a})₂]_{c}R^{2b};

each R^{2a} is independently hydrogen or C₁-C₆ alkyl;

R^{2b} is —N(L₁-B)₂; —(OCH₂CH₂)₆OH; or —(OCH₂CH₂)_{b}OCH₃;

each R³ and R⁴ is independently C₆-C₃₀ aliphatic;

each I.₃ is independently C₁-C₁₀ alkylene;

each B is independently hydrogen or an ionizable nitrogen-containing group;

each X¹ is independently a covalent bond or O;

each a is independently an integer of 1-10;

each b is independently an integer of 1-10; and

each c is independently an integer of 1-10.

In some embodiments, the lipid nanoparticle comprises a lipid having the structure:

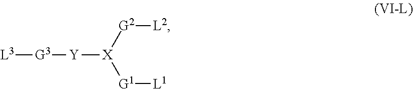

or a pharmaceutically acceptable salt, prodrug or stereoisomer thereof, wherein:

X is N, and Y is absent; or X is CR, and Y is NR;

L¹ is —O(C—O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c}, or —NR^{a}C(═O)OR¹;

L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;

L³ is —O(C═O)R³ or —(C═O)OR³;

G¹ and G² are each independently C₂-C₁₂ alkylene or C₂-C₁₂ alkenylene;

G³ is C₁-C₂₄ alkylene, C₂-C₂₄ alkenylene, C₁-C₂₄ heteroalkylene or C₂-C₂₄ heteroalkenylene when X is CR, and Y is NR; and G³ is C₁-C₂₄ heteroalkylene or C₂-C₂₄ heteroalkenylene when X is N, and Y is absent;

R^{a}, R^{b}, R^{d} and R^{c} are each independently H or C₁-C₁₂ alkyl or C₁-C₁₂ alkenyl;

R^{c} and R^{f} are each independently C₁-C₁₂ alkyl or C₂-C₁₂ alkenyl;

each R is independently H or C₁-C₁₂ alkyl;

R¹, R² and R³ are each independently C₁-C₂₄ alkyl or C₂-C₂₄ alkenyl; and x is 0, 1 or 2, and

wherein each alkyl, alkenyl, alkylene, alkenylene, heteroalkylene and heteroalkenylene is independently substituted or unsubstituted unless otherwise specified.

In some embodiments, the lipid nanoparticle comprises a lipid having the structure:

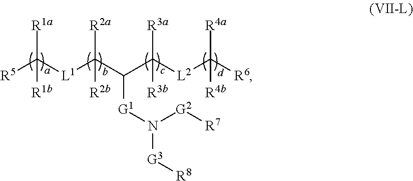

or a pharmaceutically acceptable salt, tautomer, prodrug or stereoisomer thereof, wherein:

L¹ and L² are each independently -0(C=0)-, —(C=0)0-, —C(═O)—, -0-, —S(0)x-ₛ-S—S—, —C(=0)S—, —SC(=0)-, —NR^{a}C(=0)-, —C(=0)NR^{a}—, —NR^{a}C(=0)NR^{a}—, —OC(=0)NR^{a}—, —NR^{a}C(=0)0- or a direct bond;

G¹ is C₁-C₂ alkylene, —(C=0)-, -0(C=0)-, —SC(=0)-, —NR^{a}C(=0)- or a direct bond;

G² is —C(0)-, —(CO)O—, —C(=0)S—, —C(=0)NR^{a}— or a direct bond;

G³ is C₁-C₆ alkylene;

R^{a} is H or C₁-C₁₂ alkyl;

R^{1a} and R^{1b} are, at each occurrence, independently either: (a) H or C₁-C₁₂ alkyl; or (b) R^{1a} is H or C₁-C₁₂ alkyl, and R^{Ib} together with the carbon atom to which it is bound is taken together with an adjacent R^{1b} and the carbon atom to which it is bound to form a carbon-carbon double bond;

R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;

R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond;

R^{4A} and R^{4B} are, at each occurrence, independently either: (a) H or C₁-C₁₂ alkyl; or (b) R^{4A} is H or C₁-C₁₂ alkyl, and R^{4B} together with the carbon atom to which it is bound is taken together with an adjacent R^{4B} and the carbon atom to which it is bound to form a carbon-carbon double bond;

R⁵ and R⁶ are each independently H or methyl;

R⁷ is H or C₁-C₂₀ alkyl;

R⁸ is OH, —N(R⁹)(C=0)R¹⁰, —(C=0)NR⁹R¹⁰, —NR⁹R¹⁰, —(C=0)OR″¹ or -0(C=0)R″, provided

that G³ is C₄-C₆ alkylene when R⁸ is —NR⁹R¹⁰,

R⁹ and R¹⁰ are each independently H or C₁-C₁₂ alkyl;

R″ is aralkyl;

a, b, c and d are each independently an integer from 1 to 24; and x is 0, 1 or 2,

wherein each alkyl, alkylene and aralkyl is optionally substituted.

In some embodiments, the lipid nanoparticle comprises a lipid having the structure:

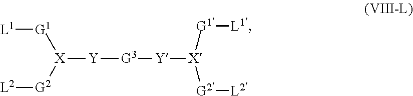

or a pharmaceutically acceptable salt, prodrug or stereoisomer thereof, wherein:

X and X′ are each independently N or CR;

Y and Y′ are each independently absent, —O(C═O)—, —(C═O)O— or NR, provided that:
a) Y is absent when X is N;
b) Y′ is absent when X′ is N;
c) Y is —O(C═O)—, —(C═O)O— or NR when X is CR; and
d) Y′ is —O(C═O)—, —(C═O)O— or NR when X′ is CR,

L¹ and L^{1′} are each independently —O(C═O)R′, —(C═O)OR′, —C(═O)R′, —OR¹, —S(O)_{z}R′, —S—SR¹, —C(═O)SR′, —SC(═O)R′, —NR^{a}C(═O)R′, —C(═O)NR^{b}R^{c}, —NR^{a}C(═O)NR^{b}R^{c}, —OC(═O)NR^{b}R′ or —NR^{a}C(═O)OR′;

L² and L^{2′} are each independently —O(C═O)R², —(C═O)OR², —C(═O)R², —OR², —S(O)_{z}R², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;

G¹. G^{1′}, G² and G^{2′} are each independently C₂-C₁₂ alkylene or C₂-C₁₂ alkenylene;

G is C₂-C₂₄ heteroalkylene or C₂-C₂₄ heteroalkenylene;

R^{a}, R^{b}, R^{d} and R^{c} are, at each occurrence, independently H, C₁-C₁₂ alkyl or C₂-C₁₂ alkenyl;

R^{c} and R^{f} are, at each occurrence, independently C₁-C₁₂ alkyl or C₂-C₁₂ alkenyl;

R is, at each occurrence, independently H or C₁-C₁₂ alkyl;

R¹ and R² are, at each occurrence, independently branched C₆-C₂₄ alkyl or branched C₆-C₂₄ alkenyl;

z is 0, 1 or 2, and wherein each alkyl, alkenyl, alkylene, alkenylene, heteroalkylene and heteroalkenylene is independently substituted or unsubstituted unless otherwise specified.

In some embodiments, the lipid nanoparticle comprises a lipid having the structure:

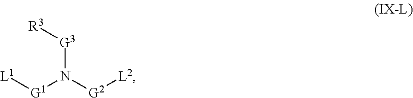

or a pharmaceutically acceptable salt, prodrug or stereoisomer thereof, wherein:

L¹ is —O(C═O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;

L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R².

G¹ and G² are each independently C₂-C₁₂ alkylene or C₂-C₁₂ alkenylene;

G³ is C₁-C₂₄ alkylene, C₂-C₂₄ alkenylene, C₃-C₈ cycloalkylene or C₃-C₈ cycloalkenylene;

R^{a}, R^{b}, R^{d} and R^{c} are each independently H or C₁-C₁₂ alkyl or C₁-C₁₂ alkenyl;

R^{c} and R^{f} are each independently C₁-C₁₂ alkyl or C₂-C₁₂ alkenyl;

R¹ and R² are each independently branched C₆-C₂₄ alkyl or branched C₆-C₂₄ alkenyl;

R³ is —N(R⁴)R⁵;

R⁴ is C₁-C₁₂ alkyl;

R⁵ is substituted C₁-C₁₂ alkyl; and

x is 0, 1 or 2, and

wherein each alkyl, alkenyl, alkylene, alkenylene, cycloalkylene, cycloalkenylene, aryl and aralkyl is independently substituted or unsubstituted unless otherwise specified.

In some embodiments, the lipid nanoparticle comprises a lipid having the structure:

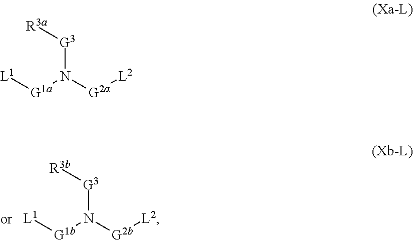

or a pharmaceutically acceptable salt, prodrug or stereoisomer thereof, wherein:

L¹ is —O(C═O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;

L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R².

G^{1a} and G^{2b} are each independently C₂-C₁₂ alkylene or C₂-C₁₂ alkenylene; G^{1b} and G^{2b} are each independently C₁-C₁₂ alkylene or C₂-C₁₂ alkenylene;

G³ is C₁-C₂₄ alkylene, C₂-C₂₄ alkenylene, C₃-C₈ cycloalkylene or C₃-C₈ cycloalkenylene;

R^{a}, R^{b}, R^{d} and R^{c} are each independently H or C₁-C₁₂ alkyl or C₂-C₁₂ alkenyl;

R^{c} and R^{f} are each independently C₁-C₁₂ alkyl or C₂-C₁₂ alkenyl;

R¹ and R² are each independently branched C₆-C₂₄ alkyl or branched C₆-C₂₄ alkenyl;

R^{3a} is —C(═O)N(R^{4a})R^{5a} or —C(═O)OR⁶;

R^{3b} is —NR^{4b}C(═O)R^{5b};

R^{4a} is C₁-C₁₂ alkyl;

R^{4b} is H, C₁-C₁₂ alkyl or C₂-C₁₂ alkenyl;

R^{5a} is H, C₁-C₈ alkyl or C₂-C₈ alkenyl;

R^{5b} is C₂-C₁₂ alkyl or C₂-C₁₂ alkenyl when R^{4b} is H; or R^{5b} is C₁-C₁₂ alkyl or C₂-C₁₂ alkenyl when R^{4b} is C₁-C₁₂ alkyl or C₂-C₁₂ alkenyl;

R⁶ is H, aryl or aralkyl; and

x is 0, 1 or 2, and

wherein each alkyl, alkenyl, alkylene, alkenylene, cycloalkylene, cycloalkenylene, aryl and aralkyl is independently substituted or unsubstituted.

In some embodiments, the lipid nanoparticle comprises a lipid having the structure:

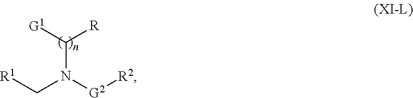

or a pharmaceutically acceptable salt, prodrug or stereoisomer thereof, wherein:

G¹ is —OH, —R³R⁴, —(C=0)R⁵ or —R³(C=0)R⁵;

G² is —CH₂— or —(C=0)-;

R is, at each occurrence, independently H or OH;

R¹ and R² are each independently optionally substituted branched, saturated or unsaturated C₁₂-C₃₆ alkyl;

R³ and R⁴ are each independently H or optionally substituted straight or branched, saturated or unsaturated C₁-C₆ alkyl;

R⁵ is optionally substituted straight or branched, saturated or unsaturated C₁-C₆ alkyl; and

n is an integer from 2 to 6.

In some embodiments, the lipid nanoparticle comprises a lipid having the structure:

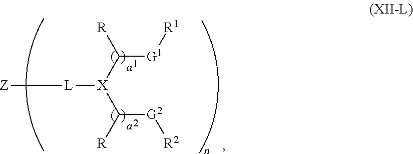

or a pharmaceutically acceptable salt, prodrug or stereoisomer thereof, wherein:

one of G¹ or G² is, at each occurrence, —O(C═O)—, —(C═O)O—, —C(═O)—, —O—, —S(O), —S—S—, —C(═O)S—, SC(═O)—, —N(R^{a})C(═O)—, —C(═O)N(R^{a})—, —N(R^{a})C(═O)N(R^{a})—, —OC(═O)N(R^{a})— or —N(R^{a})C(═O)O—, and the other of G¹ or G² is, at each occurrence, —O(C═O)—, —(C═O)O—, —C(═O)—, —O—, —S(O), —S—S—, —C(═O)S—, —SC(═O)—, —N(R^{a})C(═O)—, —C(═O)N(R^{a})—, —N(R^{a})C(═O)N(R^{a})—, —OC(═O)N(R^{a})— or —N(R^{a})C(═O)O— or a direct bond;

L is, at each occurrence, ˜O(C═O)—, wherein ˜ represents a covalent bond to X; X is CR^{a};

Z is alkyl, cycloalkyl or a monovalent moiety comprising at least one polar functional group when n is 1; or Z is alkylene, cycloalkylene or a polyvalent moiety comprising at least one polar functional group when n is greater than 1;

R^{a} is, at each occurrence, independently H, C₁-C₁₂ alkyl, C₁-C₁₂ hydroxylalkyl, C₁-C₁₂ aminoalkyl, C₁-C₁₂ alkylaminylalkyl, C₁-C₁₂ alkoxyalkyl, C₁-C₁₂ alkoxycarbonyl, C₁-C₁₂ alkylcarbonyloxy, C₁-C₁₂ alkylcarbonyloxyalkyl or C₁-C₁₂ alkylcarbonyl;

R is, at each occurrence, independently either: (a) H or C₁-C₁₂ alkyl; or (b) R together with the carbon atom to which it is bound is taken together with an adjacent R and the carbon atom to which it is bound to form a carbon-carbon double bond;

R¹ and R² have, at each occurrence, the following structure, respectively:

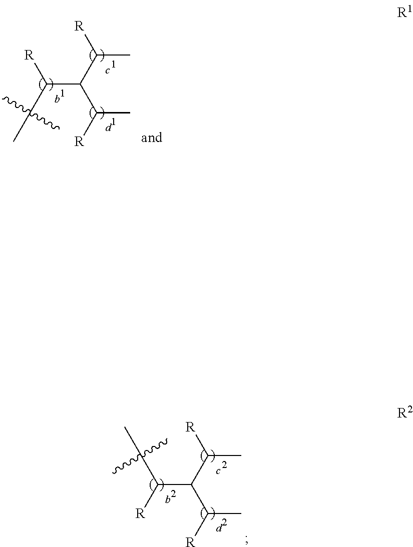

a¹ and a² are, at each occurrence, independently an integer from 3 to 12; b¹ and b² are, at each occurrence, independently 0 or 1;
c¹ and c² are, at each occurrence, independently an integer from 5 to 10; d¹ and d² are, at each occurrence, independently an integer from 5 to 10; y is, at each occurrence, independently an integer from 0 to 2; and n is an integer from 1 to 6,

wherein each alkyl, alkylene, hydroxylalkyl, aminoalkyl, alkylaminylalkyl, alkoxyalkyl, alkoxycarbonyl, alkylcarbonyloxy, alkylcarbonyloxyalkyl and alkylcarbonyl is optionally substituted with one or more substituent.

In some embodiments, the lipid nanoparticle comprises a lipid having the structure:

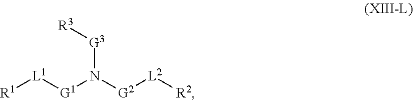

or a pharmaceutically acceptable salt, prodrug or stereoisomer thereof, wherein:

one of L¹ or L² is —O(C═O)—, —(C═O)O—, —C(═O)—, —O—, —S(O)ₓ—, —S—S—, —C(═O)S—, —SC(═O)—, —R^{a}C(═O)—, —C(═O)R^{a}—, R^{a}C(═O)R^{a}—, —OC(═O)R^{a}— or —R^{a}C(═O)O—, and the other of L¹ or L² is —O(C═O)—, —(C═O)O—, —C(═O)—, —O—, —S(O)ₓ—, —S—S—, —C(═O)S—, SC(═O)—, —R^{a}C(═O)—, —C(═O)R^{a}—, R^{a}C(═O)R^{a}—, —OC(═O)R^{a}— or —NR^{a}C(═O)O— or a direct bond;

G¹ and G² are each independently unsubstituted C₁-C₁₂ alkylene or C₁-C₁₂ alkenylene;

G³ is C₁-C₂₄ alkylene, C₁-C₂₄ alkenylene, C₃-C₈ cycloalkylene, C₃-C₈ cycloalkenylene;

R^{a} is H or C₁-C₁₂ alkyl;

R¹ and R² are each independently C₆-C₂₄ alkyl or C₆-C₂₄ alkenyl;

R³ is H, OR⁵, CN, —C(═O)OR⁴, —OC(═O)R⁴ or —R⁵C(═O)R⁴;

R⁴ is C₁-C₁₂ alkyl;

R⁵ is H or C₁-C₆ alkyl; and

x is 0, 1 or 2.

In some embodiments, the lipid nanoparticle comprises a lipid having the structure:

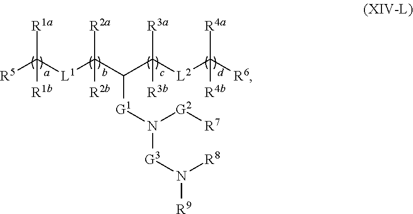

or a pharmaceutically acceptable salt, tautomer, prodrug or stereoisomer thereof, wherein:

L¹ and L² are each independently -0(C=0)-, —(C=0)0-, —C(=0)-, -0-, —S(0)ₓ—, —S—S—, —C(=0)S—, —SC(=0)-, —R^{a}C(=0)-, —C(=0)R^{a}—, —R^{a}C(=0)R^{a}—, —OC(=0)R^{a}—, —R^{a}C(=0)0- or a direct bond;

G¹ is C₁-C₂ alkylene, —(C=0)-, -0(C=0)-, —SC(=0)-, —R^{a}C(=0)- or a direct bond: G² is —C(=0)-, —(C=0)0-, —C(=0)S—, —C(=0)NR^{a}— or a direct bond;

G³ is C₁-C₆ alkylene;

R^{a} is H or C₁-C₁₂ alkyl;

R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;

R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;

R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond;

R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;

R⁵ and R⁶ are each independently H or methyl;

R⁷ is C₄-C₂₀ alkyl;

R⁸ and R⁹ are each independently C₁-C₁₂ alkyl; or R⁸ and R⁹, together with the nitrogen atom to which they are attached, form a 5, 6 or 7-membered heterocyclic ring;

a, b, c and d are each independently an integer from 1 to 24; and x is 0, 1 or 2.

In some embodiments, the lipid nanoparticle comprises a lipid having the structure:

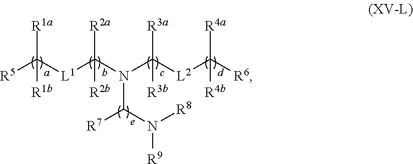

or a pharmaceutically acceptable salt, tautomer, prodrug or stereoisomer thereof, wherein:

L¹ and L² are each independently -0(C=0)-, —(C=0)0- or a carbon-carbon double bond;

R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;

R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;

R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;

R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;

R⁵ and R⁶ are each independently methyl or cycloalkyl;

R⁷ is, at each occurrence, independently H or C₁-C₁₂ alkyl; R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom;

a and d are each independently an integer from 0 to 24; b and c are each independently an integer from 1 to 24; and e is 1 or 2,

provided that:

at least one of R^{1a}, R^{2a}, R^{3a} or R^{4a} is C₁-C₁₂ alkyl, or at least one of L¹ or L² is -0(C=0)- or —(C=0)0-; and

R^{1a} and R^{1b} are not isopropyl when a is 6 or n-butyl when a is 8.

In some embodiments, the lipid nanoparticle comprises a lipid having the structure:

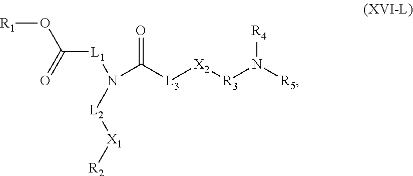

or a pharmaceutically acceptable salt thereof, wherein

R₁ and R₂ are the same or different, each a linear or branched alkyl with 1-9 carbons, or as alkenyl or alkynyl with 2 to 11 carbon atoms,

L₁ and L₂ are the same or different, each a linear alkyl having 5 to 18 carbon atoms, or form a heterocycle with N,

X₁ is a bond, or is —CG-G- whereby L2-CO—O—R₂ is formed,

X₂ is S or O,

L₃ is a bond or a lower alkyl, or form a heterocycle with N,

R₃ is a lower alkyl, and

R₄ and R₅ are the same or different, each a lower alkyl.

In some embodiments, the lipid nanoparticle comprises an ionizable lipid having the structure:

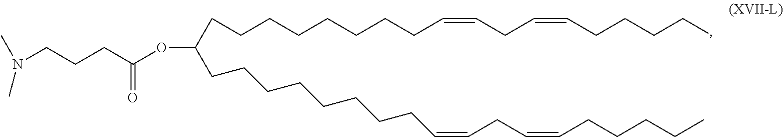

or a pharmaceutically acceptable salt thereof.

In some embodiments, the lipid nanoparticle comprises a lipid having the structure:

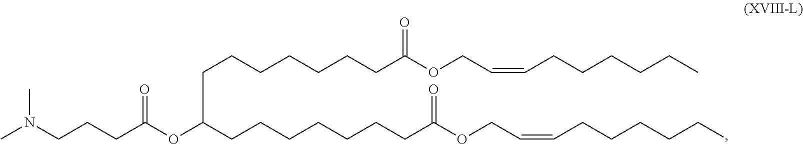

or a pharmaceutically acceptable salt thereof.

In some embodiments, the lipid nanoparticle comprises a lipid having the structure:

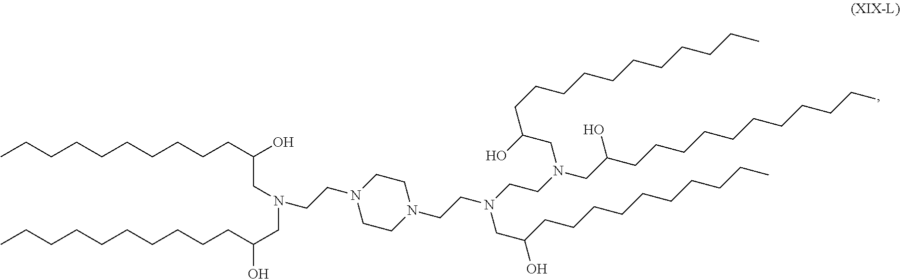

or a pharmaceutically acceptable salt thereof.

In some embodiments, the lipid nanoparticle comprises a lipid having the structure:

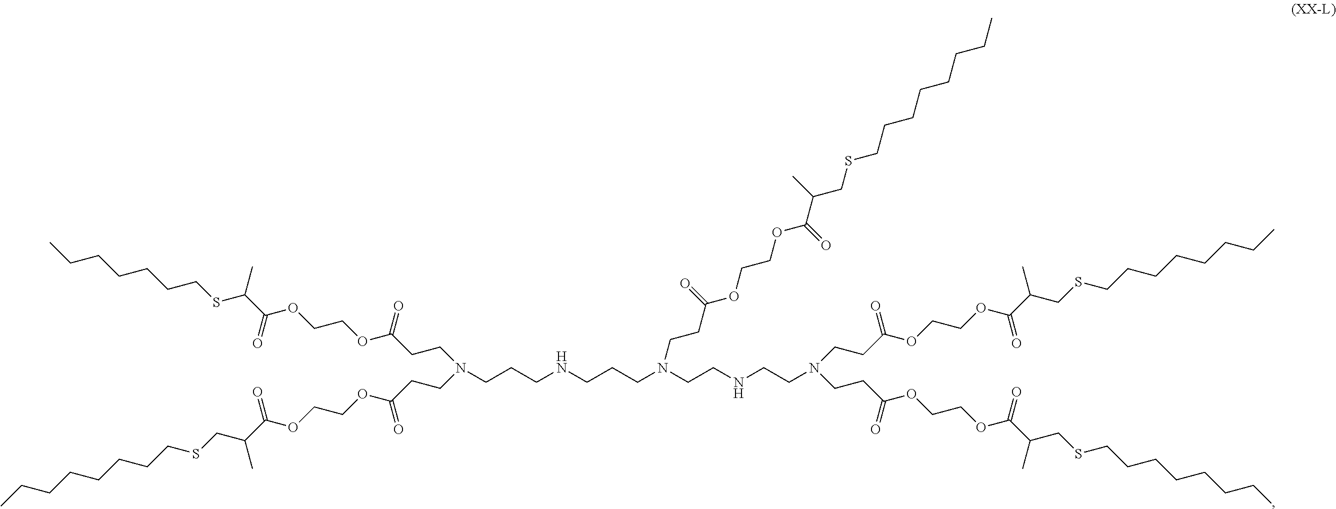

or a pharmaceutically acceptable salt thereof.

In some embodiments, the lipid nanoparticle comprises a lipid having the structure:

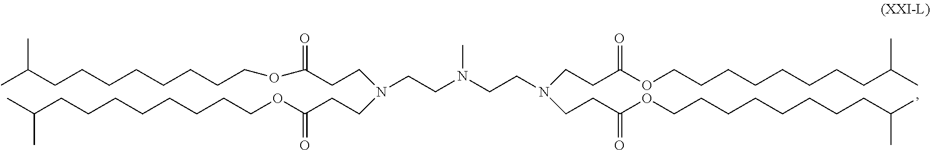

or a pharmaceutically acceptable salt thereof.

In some embodiments, the lipid nanoparticle comprises a lipid having the structure:

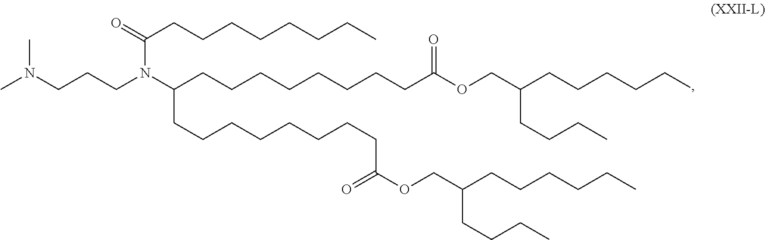

or a pharmaceutically acceptable salt thereof.

In some embodiments, the lipid nanoparticle comprises a lipid having the structure:

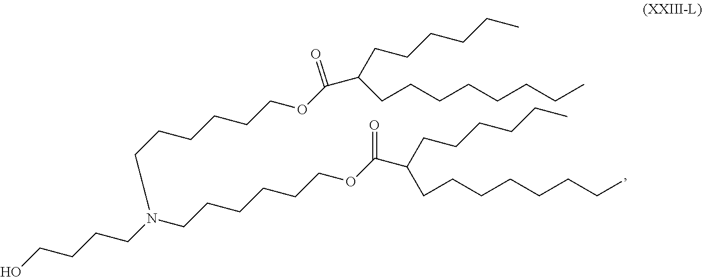

or a pharmaceutically acceptable salt thereof.

In some embodiments, the lipid nanoparticle comprises a lipid having the structure:

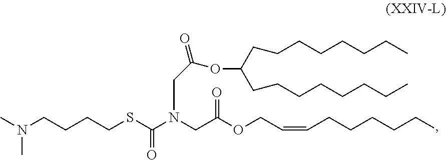

or a pharmaceutically acceptable salt thereof.

In some embodiments, the lipid nanoparticle comprises a lipid having the structure:

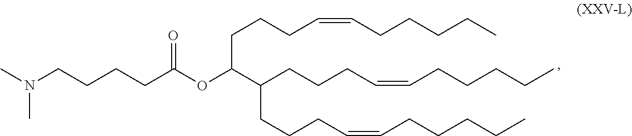 [SMILES: CCCCC/C=C\CCCC(CCC/C=C\CCCCC)C(CCC/C=C\CCCCC)OC(=O)CCCCN(C)C]

or a pharmaceutically acceptable salt thereof.

In some embodiments, the lipid nanoparticle comprises a lipid having the structure:

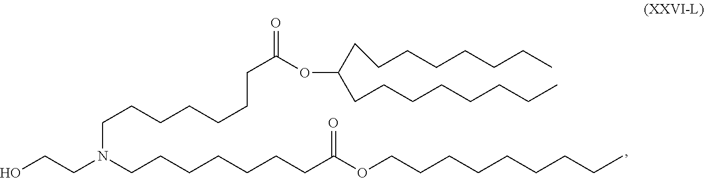 [SMILES: CCCCCCCCCOC(=O)CCCCCCCN(CCO)CCCCCCCC(=O)OC(CCCCCCCC)CCCCCCCC]

or a pharmaceutically acceptable salt thereof.

In some embodiments, the lipid nanoparticle comprises a lipid having the structure:

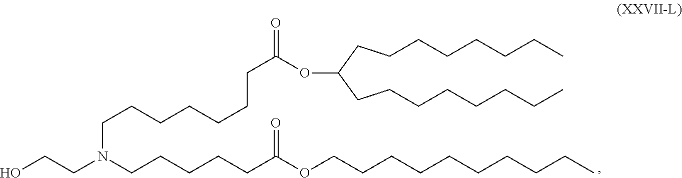 [SMILES: CCCCCCCCCCOC(=O)CCCCCN(CCO)CCCCCCCC(=O)OC(CCCCCCCC)CCCCCCCC]

or a pharmaceutically acceptable salt thereof.

### Non-Cationic Lipids

In certain embodiments, the lipid nanoparticles described herein comprise one or more non-cationic lipids. Non-cationic lipids may be phospholipids.

In some embodiments, the lipid nanoparticle comprises 5-25 mol % non-cationic lipid. For example, the lipid nanoparticle may comprise 5-20 mol %, 5-15 mol %, 5-10 mol %, 10-25 mol %, 10-20 mol %, 10-25 mol %, 15-25 mol %, 15-20 mol %, or 20-25 mol % non-cationic lipid. In some embodiments, the lipid nanoparticle comprises 5 mol %, 10 mol %, 15 mol %, 20 mol %, or 25 mol % non-cationic lipid.

In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

In some embodiments, the lipid nanoparticle comprises 5-15 mol %, 5-10 mol %, or 10-15 mol % DSPC. For example, the lipid nanoparticle may comprise 5, 6, 7, 8, 9, 10, 11, 12, 13, 14, or 15 mol % DSPC.

In certain embodiments, the lipid composition of the lipid nanoparticle composition disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, a phospholipid of the invention comprises 1,2-distearoyl-sn-glycero-3-phosphocholine (DSPC), 1,2-Distearoyl-sn-glycero-3-phosphoethanolamine (DSPE), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

In certain embodiments, a phospholipid useful or potentially useful in the present invention is an analog or variant of DSPC. In certain embodiments, a phospholipid useful or potentially useful in the present invention is a compound of Formula (IX):

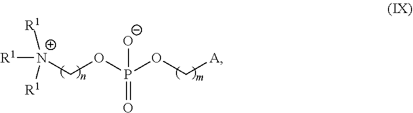

or a salt thereof, wherein:

each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;

n is 1, 2, 3, 4, 5, 6, 7, 8, 9, or 10;

m is 0, 1, 2, 3, 4, 5, 6, 7, 8, 9, or 10;

A is of the formula:

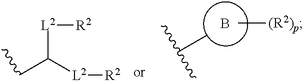

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), —NR^{N}C(O)O, or NR^{N}C(O)N(R^{N});

each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O) OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;

each instance of R^{N} is independently hydrogen, optionally substituted alkyl, or a nitrogen protecting group;

Ring B is optionally substituted carbocyclyl, optionally substituted heterocyclyl, optionally substituted aryl, or optionally substituted heteroaryl; and

p is 1 or 2;

provided that the compound is not of the formula:

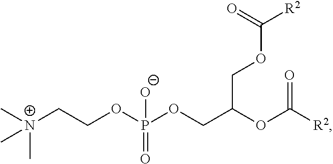

wherein each instance of R² is independently unsubstituted alkyl, unsubstituted alkenyl, or unsubstituted alkynyl.

In some embodiments, the phospholipids may be one or more of the phospholipids described in PCT Application No. PCT/US2018/037922.

In some embodiments, the lipid nanoparticle comprises a molar ratio of 5-25% non-cationic lipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% non-cationic lipid. In some embodiments, the lipid nanoparticle comprises a molar ratio of 5%, 10%, 15%, 20%, 25%, or 30% non-cationic lipid.

In some embodiments, the lipid nanoparticle comprises a molar ratio of 5-25% phospholipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% phospholipid. In some embodiments, the lipid nanoparticle comprises a molar ratio of 5%, 10%, 15%, 20%, 25%, or 30% phospholipid lipid.

### Structural Lipids

The lipid composition of a pharmaceutical composition disclosed herein can comprise one or more structural lipids. As used herein, the term “structural lipid” includes sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, the structural lipids may be one or more of the structural lipids described in U.S. application Ser. No. 16/493,814.

In some embodiments, the lipid nanoparticle comprises a molar ratio of 25-55% structural lipid relative to the other lipid components. For example, the lipid nanoparticle may comprise a molar ratio of 10-55%, 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% structural lipid. In some embodiments, the lipid nanoparticle comprises a molar ratio of 10%, 15%, 20%, 25%, 30%, 35%, 40%, 45%, 50%, or 55% structural lipid.

In some embodiments, the lipid nanoparticle comprises 30-45 mol % sterol, optionally 35-40 mol %, for example, 30-31 mol %, 31-32 mol %, 32-33 mol %, 33-34 mol %, 35-35 mol %, 35-36 mol %, 36-37 mol %, 38-38 mol %, 38-39 mol %, or 39-40 mol %. In some embodiments, the lipid nanoparticle comprises 25-55 mol % sterol. For example, the lipid nanoparticle may comprise 25-50 mol %, 25-45 mol %, 25-40 mol %, 25-35 mol %, 25-30 mol %, 30-55 mol %, 30-50 mol %, 30-45 mol %, 30-40 mol %, 30-35 mol %, 35-55 mol %, 35-50 mol %, 35-45 mol %, 35-mol %, 40-55 mol %, 40-50 mol %, 40-45 mol %, 45-55 mol %, 45-50 mol %, or 50-55 mol % sterol. In some embodiments, the lipid nanoparticle comprises 25 mol %, 30 mol %, 35 mol %, 40 mol %, 45 mol %, 50 mol %, or 55 mol % sterol.

In some embodiments, the lipid nanoparticle comprises 35-40 mol % cholesterol. For example, the lipid nanoparticle may comprise 35, 35.5, 36, 36.5, 37, 37.5, 38, 38.5, 39, 39.5, or mol % cholesterol.

### Polyethylene Glycol (PEG)-Lipids

The lipid composition of a pharmaceutical composition disclosed herein can comprise one or more polyethylene glycol (PEG) lipids.

As used herein, the term “PEG-lipid” or “PEG-modified lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines, and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In some embodiments, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG, and/or PEG-DPG.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In some embodiments, the PEG-lipid is PEG₂ₖ-DMG.

In some embodiments, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

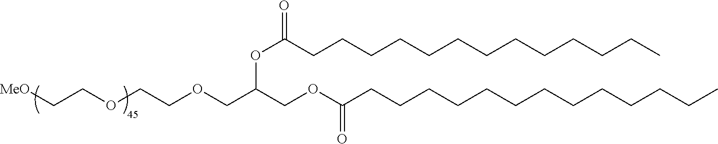

In some embodiments, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present invention.

In certain embodiments, a PEG lipid useful in the present invention is a compound of Formula (X):

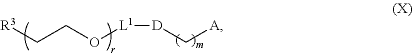

or salts thereof, wherein:

R³ is —OR^{O};

R^{O} is hydrogen, optionally substituted alkyl, or an oxygen protecting group;

r is an integer between 1 and 100, inclusive;

L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});

D is a moiety obtained by click chemistry or a moiety cleavable under physiological conditions;

m is 0, 1, 2, 3, 4, 5, 6, 7, 8, 9, or 10;

A is of the formula:

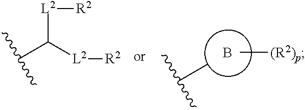

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), —NR^{N}C(O)O, or NR^{N}C(O)N(R^{N});

each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O) OS(O) S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;

each instance of R^{N} is independently hydrogen, optionally substituted alkyl, or a nitrogen protecting group;

Ring B is optionally substituted carbocyclyl, optionally substituted heterocyclyl, optionally substituted aryl, or optionally substituted heteroaryl; and

p is 1 or 2.

In certain embodiments, the compound of Formula (X) is a PEG-OH lipid (i.e., R³ is —OR^{O}, and R^{O} is hydrogen). In certain embodiments, the compound of Formula (X) is of Formula (X—OH):

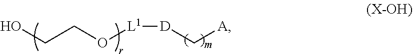

or a salt thereof.

In certain embodiments, a PEG lipid useful in the present invention is a PEGylated fatty acid. In certain embodiments, a PEG lipid useful in the present invention is a compound of Formula (XI). Provided herein are compounds of Formula (XI):

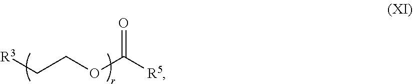

or a salts thereof, wherein:

R³ is —OR^{O};

R^{O} is hydrogen, optionally substituted alkyl or an oxygen protecting group;
r is an integer between 1 and 100, inclusive;

R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), —C(O)N(R^{N}), NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), —C(S)N(R^{N}), NR^{N}C(S), NR^{N}C(S)N(R^{N}), S(O), OS(O), S(O)O, OS(O)O, OS(O)₂, S(O)₂O, —OS(O)₂O, N(R^{N})S(O), S(O)N(R^{N}), N(R^{N})S(O)N(R^{N}), OS(O)N(R^{N}), N(R^{N})S(O)O, S(O)₂, —N(R^{N})S(O)₂, S(O)₂N(R^{N}), N(R^{N})S(O)₂N(R^{N}), OS(O)₂N(R^{N}), or N(R^{N})S(O)₂O; and

each instance of R^{N} is independently hydrogen, optionally substituted alkyl, or a nitrogen protecting group.

In certain embodiments, the compound of Formula (XI) is of Formula (XI-OH):

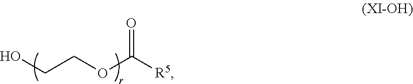

or a salt thereof. In some embodiments, r is 40-50.

In yet other embodiments the compound of Formula (XI) is:

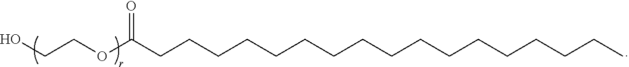

or a salt thereof.

In some embodiments, the compound of Formula (XI) is

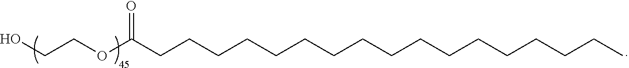

In some embodiments, the lipid composition of the pharmaceutical compositions disclosed herein does not comprise a PEG-lipid.

In some embodiments, the PEG-lipids may be one or more of the PEG lipids described in U.S. Application No. U.S. Ser. No. 15/674,872.

In some embodiments, the lipid nanoparticle comprises a molar ratio of 0.5-15% PEG lipid relative to the other lipid components. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15% PEG lipid. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-lipid.

In some embodiments, the lipid nanoparticle comprises 1-5% PEG-modified lipid, optionally 1-3 mol %, for example 1.5 to 2.5 mol %, 1-2 mol %, 2-3 mol %, 3-4 mol %, or 4-5 mol %. In some embodiments, the lipid nanoparticle comprises 0.5-15 mol % PEG-modified lipid. For example, the lipid nanoparticle may comprise 0.5-10 mol %, 0.5-5 mol %, 1-15 mol %, 1-10 mol %, 1-5 mol %, 2-15 mol %, 2-10 mol %, 2-5 mol %, 5-15 mol %, 5-10 mol %, or 10-15 mol %. In some embodiments, the lipid nanoparticle comprises 0.5 mol %, 1 mol %, 2 mol %, 3 mol %, 4 mol %, 5 mol %, 6 mol %, 7 mol %, 8 mol %, 9 mol %, 10 mol %, 11 mol %, 12 mol %, 13 mol %, 14 mol %, or 15 mol % PEG-modified lipid.

In some embodiments, the lipid nanoparticle comprises 20-60 mol % ionizable amino lipid, 5-25 mol % non-cationic lipid, 25-55 mol % sterol, and 0.5-15 mol % PEG-modified lipid.

In some embodiments, a LNP of the disclosure comprises an ionizable amino lipid of Compound 1, wherein the non-cationic lipid is DSPC, the structural lipid that is cholesterol, and the PEG lipid is DMG-PEG.

In some embodiments, a LNP of the invention comprises an ionizable amino lipid of any of Formula VI, VII or VIIII, a phospholipid comprising DSPC, a structural lipid, and a PEG lipid comprising PEG-DMG.

In some embodiments, a LNP of the invention comprises an ionizable amino lipid of any of Formula VI, VII or VIII, a phospholipid comprising DSPC, a structural lipid, and a PEG lipid comprising a compound having Formula XI.

In some embodiments, a LNP of the invention comprises an ionizable amino lipid of Formula VI, VII or VIII, a phospholipid comprising a compound having Formula VIII, a structural lipid, and the PEG lipid comprising a compound having Formula X or XI.

In some embodiments, a LNP of the invention comprises an ionizable amino lipid of Formula VI, VII or VIII, a phospholipid comprising a compound having Formula IX, a structural lipid, and the PEG lipid comprising a compound having Formula X or XI.

In some embodiments, a LNP of the invention comprises an ionizable amino lipid of Formula VI, VII or VIII, a phospholipid having Formula IX, a structural lipid, and a PEG lipid comprising a compound having Formula XI.

In some embodiments, the lipid nanoparticle comprises 49 mol % ionizable amino lipid, mol % DSPC, 38.5 mol % cholesterol, and 2.5 mol % DMG-PEG.

In some embodiments, the lipid nanoparticle comprises 49 mol % ionizable amino lipid, 11 mol % DSPC, 38.5 mol % cholesterol, and 1.5 mol % DMG-PEG.

In some embodiments, the lipid nanoparticle comprises 48 mol % ionizable amino lipid, 11 mol % DSPC, 38.5 mol % cholesterol, and 2.5 mol % DMG-PEG.

In some embodiments, a LNP of the invention comprises an N:P ratio of from about 2:1 to about 30:1.

In some embodiments, a LNP of the invention comprises an N:P ratio of about 6:1.

In some embodiments, a LNP of the invention comprises an N:P ratio of about 3:1, 4:1, or 5:1.

In some embodiments, a LNP of the invention comprises a wt/wt ratio of the ionizable amino lipid component to the RNA of from about 10:1 to about 100:1.

In some embodiments, a LNP of the invention comprises a wt/wt ratio of the ionizable amino lipid component to the RNA of about 20:1.

In some embodiments, a LNP of the invention comprises a wt/wt ratio of the ionizable amino lipid component to the RNA of about 10:1.

Some embodiments comprise a composition having one or more LNPs having a diameter of about 150 nm or less, such as about 140 nm, 130 nm, 120 nm, 110 nm, 100 nm, 90 nm, 80 nm, 70 nm, 60 nm, 50 nm, 40 nm, 30 nm, or 20 nm or less. Some embodiments comprise a composition having a mean LNP diameter of about 150 nm or less, such as about 140 nm, 130 nm, 120 nm, 110 nm, 100 nm, 90 nm, 80 nm, 70 nm, 60 nm, 50 nm, 40 nm, 30 nm, or 20 nm or less. In some embodiments, the composition has a mean LNP diameter from about 30 nm to about 150 nm, or a mean diameter from about 60 nm to about 120 nm.

A LNP may comprise or one or more types of lipids, including but not limited to amino lipids (e.g., ionizable amino lipids), neutral lipids, non-cationic lipids, charged lipids, PEG-modified lipids, phospholipids, structural lipids and sterols. In some embodiments, a LNP may further comprise one or more cargo molecules, including but not limited to nucleic acids (e.g., mRNA, plasmid DNA, DNA or RNA oligonucleotides, siRNA, shRNA, snRNA, snoRNA, lncRNA, etc.), small molecules, proteins and peptides.

In some embodiments, the composition comprises a liposome. A liposome is a lipid particle comprising lipids arranged into one or more concentric lipid bilayers around a central region. The central region of a liposome may comprises an aqueous solution, suspension, or other aqueous composition.

In some embodiments, a lipid nanoparticle may comprise two or more components (e.g., amino lipid and nucleic acid, PEG-lipid, phospholipid, structural lipid). For instance, a lipid nanoparticle may comprise an amino lipid and a nucleic acid. Compositions comprising the lipid nanoparticles, such as those described herein, may be used for a wide variety of applications, including the stealth delivery of therapeutic payloads with minimal adverse innate immune response.

Effective in vivo delivery of nucleic acids represents a continuing medical challenge. Exogenous nucleic acids (i.e., originating from outside of a cell or organism) are readily degraded in the body, e.g., by the immune system. Accordingly, effective delivery of nucleic acids to cells often requires the use of a particulate carrier (e.g., lipid nanoparticles). The particulate carrier should be formulated to have minimal particle aggregation, be relatively stable prior to intracellular delivery, effectively deliver nucleic acids intracellularly, and illicit no or minimal immune response. To achieve minimal particle aggregation and pre-delivery stability, many conventional particulate carriers have relied on the presence and/or concentration of certain components (e.g., PEG-lipid). However, it has been discovered that certain components may decrease the stability of encapsulated nucleic acids (e.g., mRNA molecules). The reduced stability may limit the broad applicability of the particulate carriers. As such, there remains a need for methods by which to improve the stability of nucleic acid (e.g., mRNA) encapsulated within lipid nanoparticles.

In some embodiments, the lipid nanoparticles comprise one or more of ionizable molecules, polynucleotides, and optional components, such as structural lipids, sterols, neutral lipids, phospholipids and a molecule capable of reducing particle aggregation (e.g., polyethylene glycol (PEG), PEG-modified lipid), such as those described above.

In some embodiments, a LNP described herein may include one or more ionizable molecules (e.g., amino lipids or ionizable lipids). The ionizable molecule may comprise a charged group and may have a certain pKa. In certain embodiments, the pKa of the ionizable molecule may be greater than or equal to about 6, greater than or equal to about 6.2, greater than or equal to about 6.5, greater than or equal to about 6.8, greater than or equal to about 7, greater than or equal to about 7.2, greater than or equal to about 7.5, greater than or equal to about 7.8, greater than or equal to about 8. In some embodiments, the pKa of the ionizable molecule may be less than or equal to about 10, less than or equal to about 9.8, less than or equal to about 9.5, less than or equal to about 9.2, less than or equal to about 9.0, less than or equal to about 8.8, or less than or equal to about 8.5. Combinations of the above referenced ranges are also possible (e.g., greater than or equal to 6 and less than or equal to about 8.5). Other ranges are also possible. In embodiments in which more than one type of ionizable molecule are present in a particle, each type of ionizable molecule may independently have a pKa in one or more of the ranges described above.

In general, an ionizable molecule comprises one or more charged groups. In some embodiments, an ionizable molecule may be positively charged or negatively charged. For instance, an ionizable molecule may be positively charged. For example, an ionizable molecule may comprise an amine group. As used herein, the term “ionizable molecule” has its ordinary meaning in the art and may refer to a molecule or matrix comprising one or more charged moiety. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidizolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule and/or matrix may be selected as desired.

In some cases, an ionizable molecule (e.g., an amino lipid or ionizable lipid) may include one or more precursor moieties that can be converted to charged moieties. For instance, the ionizable molecule may include a neutral moiety that can be hydrolyzed to form a charged moiety, such as those described above. As a non-limiting specific example, the molecule or matrix may include an amide, which can be hydrolyzed to form an amine, respectively. Those of ordinary skill in the art will be able to determine whether a given chemical moiety carries a formal electronic charge (for example, by inspection, pH titration, ionic conductivity measurements, etc.), and/or whether a given chemical moiety can be reacted (e.g., hydrolyzed) to form a chemical moiety that carries a formal electronic charge.

The ionizable molecule (e.g., amino lipid or ionizable lipid) may have any suitable molecular weight. In certain embodiments, the molecular weight of an ionizable molecule is less than or equal to about 2,500 g/mol, less than or equal to about 2,000 g/mol, less than or equal to about 1,500 g/mol, less than or equal to about 1,250 g/mol, less than or equal to about 1,000 g/mol, less than or equal to about 900 g/mol, less than or equal to about 800 g/mol, less than or equal to about 700 g/mol, less than or equal to about 600 g/mol, less than or equal to about 500 g/mol, less than or equal to about 400 g/mol, less than or equal to about 300 g/mol, less than or equal to about 200 g/mol, or less than or equal to about 100 g/mol. In some instances, the molecular weight of an ionizable molecule is greater than or equal to about 100 g/mol, greater than or equal to about 200 g/mol, greater than or equal to about 300 g/mol, greater than or equal to about 400 g/mol, greater than or equal to about 500 g/mol, greater than or equal to about 600 g/mol, greater than or equal to about 700 g/mol, greater than or equal to about 1000 g/mol, greater than or equal to about 1,250 g/mol, greater than or equal to about 1,500 g/mol, greater than or equal to about 1,750 g/mol, greater than or equal to about 2,000 g/mol, or greater than or equal to about 2,250 g/mol. Combinations of the above ranges (e.g., at least about 200 g/mol and less than or equal to about 2,500 g/mol) are also possible. In embodiments in which more than one type of ionizable molecules are present in a particle, each type of ionizable molecule may independently have a molecular weight in one or more of the ranges described above.

In some embodiments, the percentage (e.g., by weight, or by mole) of a single type of ionizable molecule (e.g., amino lipid or ionizable lipid) and/or of all the ionizable molecules within a particle may be greater than or equal to about 15%, greater than or equal to about 16%, greater than or equal to about 17%, greater than or equal to about 18%, greater than or equal to about 19%, greater than or equal to about 20%, greater than or equal to about 21%, greater than or equal to about 22%, greater than or equal to about 23%, greater than or equal to about 24%, greater than or equal to about 25%, greater than or equal to about 30%, greater than or equal to about 35%, greater than or equal to about 40%, greater than or equal to about 42%, greater than or equal to about 45%, greater than or equal to about 48%, greater than or equal to about 50%, greater than or equal to about 52%, greater than or equal to about 55%, greater than or equal to about 58%, greater than or equal to about 60%, greater than or equal to about 62%, greater than or equal to about 65%, or greater than or equal to about 68%. In some instances, the percentage (e.g., by weight, or by mole) may be less than or equal to about 70%, less than or equal to about 68%, less than or equal to about 65%, less than or equal to about 62%, less than or equal to about 60%, less than or equal to about 58%, less than or equal to about 55%, less than or equal to about 52%, less than or equal to about 50%, or less than or equal to about 48%. Combinations of the above referenced ranges are also possible (e.g., greater than or equal to 20% and less than or equal to about 60%, greater than or equal to 40% and less than or equal to about 55%, etc.). In embodiments in which more than one type of ionizable molecule is present in a particle, each type of ionizable molecule may independently have a percentage (e.g., by weight, or by mole) in one or more of the ranges described above. The percentage (e.g., by weight, or by mole) may be determined by extracting the ionizable molecule(s) from the dried particles using, e.g., organic solvents, and measuring the quantity of the agent using high pressure liquid chromatography (i.e., HPLC), liquid chromatography-mass spectrometry (LC-MS), nuclear magnetic resonance (NMR), or mass spectrometry (MS). Those of ordinary skill in the art would be knowledgeable of techniques to determine the quantity of a component using the above-referenced techniques. For example, HPLC may be used to quantify the amount of a component, by, e.g., comparing the area under the curve of a HPLC chromatogram to a standard curve.

It should be understood that the terms “charged” or “charged moiety” does not refer to a “partial negative charge” or “partial positive charge” on a molecule. The terms “partial negative charge” and “partial positive charge” are given their ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

According to the disclosures herein, a lipid composition may comprise one or more lipids as described herein. Such lipids may include those useful in the preparation of lipid nanoparticle formulations as described above or as known in the art.

The term “pure” as used herein refers to material that has only the target nucleic acid active agents such that the presence of unrelated nucleic acids is reduced or eliminated, i.e., impurities or contaminants, including RNA fragments, double stranded RNA, and reverse complement impurities. For example, a purified RNA sample includes one or more target or test nucleic acids but is preferably substantially free of other nucleic acids detectable by methods described herein. As used herein, the term “substantially free” is used operationally, in the context of analytical testing of the material. Preferably, purified material is substantially free of one or more impurities or contaminants including the reverse complement transcription products and/or cytokine-inducing RNA contaminant described herein and for instance is at least 50%, 55%, 60%, 63%, 65%, 70%, 75%, 80%, 90%, 91%, 92%, 93%, 94%, 95%, 96%, or 97% pure; more preferably, at least 98% pure, and more preferably still at least 99% pure. In some embodiments a pure RNA (e.g., mRNA) sample is comprised of 100% of the target or test RNAs and includes no other RNA. In certain embodiments, the nucleic acid (e.g., mRNA) is not self-replicating RNA.

As used herein, the term “intact” refers to material (e.g., RNA, such as mRNA) that is full length (i.e., does not include fragments). In some embodiments, the intact material (e.g., RNA, such as mRNA) is pure RNA.

The purity of a composition may be characterized based on the presence of impurities in the composition at any particular point in time. Impurities include, for instance, lipid-RNA adducts, which are typical degradation products of mRNA-LNPs or elemental metals. In some embodiments, a composition is considered to have an adequate purity if less than 10% of the RNA in a composition is in the form of a lipid-RNA adduct. In some embodiments, a composition is considered to have an adequate purity if less than 9%, 8%, 7%, 6%, 5%, 4%, 3%, 2%, 1%, 0.5%, or 0.1% of the RNA in a composition is in the form of a lipid-RNA adduct.

According to the present disclosure, the term “elemental metal” is given its ordinary meaning in the art. A metal is an element that readily forms positive ions (i.e., cations) and forms metallic bonds. An elemental metal refers to a metal which is not present in a salt form or otherwise within a compound. Those of ordinary skill in the art will, in general, recognize elemental metals.

Purity can be determined by any suitable method known in the art. Non-limiting examples of methods to determine the purity of a compound include melting point determination, boiling point determination, spectroscopy (e.g., UV-VIS spectroscopy), titration, chromatography (e.g., liquid chromatography or gas chromatography, such as anion exchange chromatography, high performance liquid chromatography (HPLC), or reversed-phase ultra high-performance liquid chromatography (RP-UHPLC)), mass spectrometry, capillary electrophoresis, and optical rotation. In some embodiments, the percentage of intact RNA is determined by performing HPLC or RP-UHPLC and integrating the area under the curve (AUC) of all RNA peaks (including products shorter than the full-length product and the full-length product) and taking the main peak (representative of full length RNA) as an area percent of the total peak area.

According to some embodiments, compositions (e.g., liquid pharmaceutical compositions) disclosed herein are formulated in aqueous solutions. An aqueous solution is a solution in which components are dissolved or otherwise dispersed within water or an aqueous buffer solution.

In some embodiments, an aqueous solution disclosed herein has a given pH value. In some embodiments, the pH of an aqueous solution disclosed herein is within the range of about 4.5 to about 8.5. In some embodiments, the pH of an aqueous solution is within the range of about 5 to about 8, about 6 to about 8, about 7 to about 8, about 6.5 to about 8, about 6.5 to about 7.5, about 6.5 to about 7, about 7.5 to about 8.5, or any range or combination thereof. In some embodiments, the pH of an aqueous solution is or is about 5, is or is about 5.5, is or is about 6, is or is about 6.5, is or is about 7, is or is about 7.4, is or is about 7.5, or is or is about 8.

In some embodiments, an aqueous solution disclosed herein comprises a pH buffer component, such as a phosphate buffer, a tris buffer, an acetate buffer, a histidine buffer or a citrate buffer, among others. Such a buffer acts to modulate the pH of an aqueous solution, such as an aqueous solution having a pH of 5, 5.5, 6, 6.5, 7, 7.4, 7.5 or 8.

Aqueous solutions may comprise various concentrations of salts (e.g., buffer salts, sucrose, NaCl, etc.). In some embodiments, an aqueous solution may comprise a salt (e.g., NaCl) in a concentration of or about 50 mM, of or about 60 mM, of or about 70 mM, of or about 80 mM, of or about 90 mM, of or about 100 mM, of or about 110 mM, of or about 120 mM, of or about 130 mM, of or about 140 mM, of or about 150 mM, of or about 160 mM, of or about 170 mM, of or about 180 mM, of or about 190 mM, of or about 200 mM, or any intermediate concentration therein. In embodiments in which an aqueous solution comprises more than one salt, each salt may independently have a concentration of one or more of the values described above.

In some embodiments, the article comprises a container. In certain cases, the container houses the liquid pharmaceutical composition. In some embodiments, the article and/or the container comprises a vial, a syringe, a cartridge, an infusion pump, and/or a light protective container.

In certain embodiments, the article and/or the container comprises a label (e.g., a label on the container). In accordance with certain embodiments, the label identifies a number of individual doses of the liquid pharmaceutical composition housed in the container, an amount of each individual dose of the liquid pharmaceutical composition to be administered to a subject, and/or an effective dose of RNA within the liquid pharmaceutical composition within each individual dose.

In some instances, the label indicates appropriate storage conditions for the article and/or container. For example, in some cases, the label indicates that the article should not be stored at the glass transition temperature of the composition (e.g., liquid pharmaceutical composition). Without wishing to be bound by theory, it is believed that the stability of the RNA (e.g., mRNA) is lowest at the glass transition temperature. As used herein, the glass transition temperature is the temperature at which an amorphous substance transitions from a hard and relatively brittle (“glassy”) state into a rubbery or viscous state.

In some embodiments, the glass transition temperature of the composition is greater than or equal to −50° C., greater than or equal to −45° C., greater than or equal to −40° C., or greater than or equal to −35° C. In certain cases, the glass transition temperature of the composition is less than or equal to −20° C., less than or equal to −25° C., less than or equal to −30° C., less than or equal to −35° C., or less than or equal to −40° C. Combinations of these ranges are also possible (e.g., greater than or equal to −50° C. and less than or equal to −20° C., greater than or equal to −45° C. and less than or equal to −30° C., or greater than or equal to −35° C. and less than or equal to −30° C.).

In certain embodiments, the label indicates that the article should not be stored at a particular temperature. For example, in some instances, the label indicates that the article should not be stored at a temperature of greater than or equal to −70° C., greater than or equal to −50° C., greater than or equal to −45° C., greater than or equal to −40° C., or greater than or equal to −35° C. In certain cases, the label indicates that the article should not be stored at a temperature of less than or equal to −20° C., less than or equal to −25° C., less than or equal to −30° C., less than or equal to −35° C., or less than or equal to −40° C. Combinations of these ranges are also possible (e.g., greater than or equal to −50° C. and less than or equal to −20° C., greater than or equal to −45° C. and less than or equal to −30° C., greater than or equal to −35° C. and less than or equal to −30° C., or greater than or equal to −40° C. and less than or equal to −20° C.).

According to some embodiments, the label suggests an amount of the liquid pharmaceutical composition to be administered to a subject. In certain embodiments, the amount is greater than or equal to (1+the fraction of the RNA that would degrade in the liquid pharmaceutical composition over the shelf-life of the article)×(an individual dose of the liquid pharmaceutical composition). For example, if the shelf-life of the article were 3 months at 5° C., and if 10% (or 0.1) of the RNA in the liquid pharmaceutical composition would degrade after 3 months stored at 5° C., then the amount is greater than or equal to (1+0.1)×(an individual dose of the liquid pharmaceutical composition). For example, if the individual dose of the liquid pharmaceutical composition was 100 micrograms, then the amount would be greater than or equal to 110 micrograms.

In some embodiments, the amount is greater than or equal to (1+the fraction of the RNA that would have degraded in the liquid pharmaceutical composition at the time of administration)×(an individual dose of the liquid pharmaceutical composition). For example, if the RNA in the liquid pharmaceutical composition degrades at a rate of 10% (or 0.1) per month at 5° C., then the label would suggest administering greater than or equal to (1+0.1)×(an individual dose of the liquid pharmaceutical composition) after 1 month of storage at 5° C., greater than or equal to (1+0.2)×(an individual dose of the liquid pharmaceutical composition) after 2 months of storage at 5° C., and/or greater than or equal to (1+0.3)×(an individual dose of the liquid pharmaceutical composition) after 3 months of storage at 5° C.

The fraction of the RNA (e.g., mRNA) that would degrade in the liquid pharmaceutical composition (e.g., over the shelf-life of the article or by the time of administration) is determined by the rate of decay (wherein the rate of decay is degradation over time) of the RNA (e.g., mRNA) in given conditions (e.g., at a particular temperature, such as 5° C.) and the amount of time. The rate of decay and/or the fraction of the RNA (e.g., mRNA) that degrades may be measured as a decrease in purity over time (e.g., an increase in mRNA fragments or a decrease in intact mRNA). Purity may be measured by reverse phase HPLC.

In some embodiments, the degradation follows first order kinetics. For example, in certain cases, degradation follows the following equation:

P(t)=P(0)e^{-kt}

where P(0) is percent mRNA purity at time 0, t is the number of months after time 0, P(t) is the percent mRNA purity at time t, and k is the fraction of the mRNA that would degrade in one month in the given conditions. For example, if 1.7% of the mRNA would degrade in 1 month at the given conditions (e.g., at 5° C.) then k would be 0.017. If the purity were 100% at time 0 (so P(0) is 100%) and the product would no longer be effective if the purity of the mRNA dropped below 50% (P(t) is 50%), then the amount of time that the product could be kept in those conditions (e.g., 5° C.) and still be effective could be determined as follows: t=ln (50%/100%)/−0.027=40 months. In cases where P(0) is not 100%, P(0) may artificially be set as 100% and P(t) may be normalized accordingly.

In certain embodiments, the rate of decay of the RNA (e.g., mRNA) at a given temperature (e.g., any temperature disclosed herein) (e.g., −70° C., −40° C., −20° C., 5° C., and/or 25° C.) is greater than or equal to 0.1%/month, greater than or equal to 0.5%/month, greater than or equal to 1%/month, greater than or equal to 3%/month, greater than or equal to 5%/month, greater than or equal to 7%/month, greater than or equal to 8%/month, greater than or equal to 9%/month, greater than or equal to 10%/month, greater than or equal to 12%/month, greater than or equal to 20%/month, greater than or equal to 30%/month, greater than or equal to 40%/month, or greater than or equal to 50%/month. In some embodiments, the rate of decay of the RNA (e.g., mRNA) at a given temperature (e.g., any temperature disclosed herein) (e.g., −70° C., −40° C., −20° C., 5° C., and/or 25° C.) is less than or equal to 60%/month, less than or equal to 50%/month, less than or equal to 40%/month, less than or equal to 30%/month, less than or equal to 20%/month, less than or equal to 15%/month, less than or equal to 12%/month, less than or equal to 11%/month, less than or equal to 10%/month, less than or equal to 9%/month, less than or equal to 8%/month, less than or equal to 5%/month, less than or equal to 3%/month, less than or equal to 2%/month, or less than or equal to 1%/month. Combinations of these ranges are also possible (e.g., greater than or equal to 0.1%/month and less than or equal to 60%/month, greater than or equal to 1%/month and less than or equal to 15%/month, greater than or equal to 7%/month and less than or equal to 11%/month, or greater than or equal to 8%/month and less than or equal to 10%/month).

For example, in some cases, the rate of decay of the RNA at −70° C. and/or −40° C. is greater than or equal to 0.1%/month and less than or equal to 5%/month or greater than or equal to 0.1%/month and less than or equal to 1%/month. As another example, in certain instances, the rate of decay of the RNA at −20° C. is greater than or equal to 0.1%/month and less than or equal to 8%/month, greater than or equal to 0.5%/month and less than or equal to 5%/month, or greater than or equal to 1%/month and less than or equal to 3%/month. As yet another example, in some instances, the rate of decay of the RNA at 25° C. is greater than or equal to 10%/month and less than or equal to 60%/month, greater than or equal to 30%/month and less than or equal to 60%/month, or greater than or equal to 50%/month and less than or equal to 60%/month).

In certain embodiments, the rate of decay of the RNA (e.g., mRNA) at greater than or equal to 0° C. and less than or equal to 10° C. (e.g., 5° C.) is greater than or equal to 1%/month, greater than or equal to 3%/month, greater than or equal to 5%/month, greater than or equal to 7%/month, greater than or equal to 8%/month, greater than or equal to 9%/month, greater than or equal to 10%/month, or greater than or equal to 12%/month. In some embodiments, the rate of decay of the RNA (e.g., mRNA) at greater than or equal to 0° C. and less than or equal to 10° C. (e.g., 5° C.) is less than or equal to 15%/month, less than or equal to 12%/month, less than or equal to 11%/month, less than or equal to 10%/month, less than or equal to 9%/month, less than or equal to 8%/month, less than or equal to 5%/month, or less than or equal to 3%/month. Combinations of these ranges are also possible (e.g., greater than or equal to 1%/month and less than or equal to 15%/month, greater than or equal to 7%/month and less than or equal to 11%/month, or greater than or equal to 8%/month and less than or equal to 10%/month).

In some embodiments, the amount is greater than or equal to 1.05×(an individual dose of the liquid pharmaceutical composition), greater than or equal to 1.07×(an individual dose of the liquid pharmaceutical composition), greater than or equal to 1.08×(an individual dose of the liquid pharmaceutical composition), greater than or equal to 1.10×(an individual dose of the liquid pharmaceutical composition), greater than or equal to 1.15×(an individual dose of the liquid pharmaceutical composition), greater than or equal to 1.2×(an individual dose of the liquid pharmaceutical composition), greater than or equal to 1.25×(an individual dose of the liquid pharmaceutical composition), or greater than or equal to 1.3×(an individual dose of the liquid pharmaceutical composition). In certain embodiments, the amount is less than or equal to 2.00×(an individual dose of the liquid pharmaceutical composition), less than or equal to 1.8×(an individual dose of the liquid pharmaceutical composition), less than or equal to 1.6×(an individual dose of the liquid pharmaceutical composition), less than or equal to 1.4×(an individual dose of the liquid pharmaceutical composition), less than or equal to 1.3×(an individual dose of the liquid pharmaceutical composition), less than or equal to 1.25×(an individual dose of the liquid pharmaceutical composition), less than or equal to 1.2×(an individual dose of the liquid pharmaceutical composition), or less than or equal to 1.1×(an individual dose of the liquid pharmaceutical composition). Combinations of these ranges are also possible (e.g., greater than or equal to 1.05×(an individual dose of the liquid pharmaceutical composition) and less than or equal to 2.00×(an individual dose of the liquid pharmaceutical composition) or greater than or equal to 1.2×(an individual dose of the liquid pharmaceutical composition) and less than or equal to 1.3×(an individual dose of the liquid pharmaceutical composition)).

In accordance with certain embodiments, the container comprises a total amount of RNA (e.g., mRNA). In some cases, the total amount of RNA (e.g., mRNA) comprises greater than or equal to 40%, greater than or equal to 45%, greater than or equal to 50%, greater than or equal to 55%, greater than or equal to 60%, greater than or equal to 65%, greater than or equal to 70%, greater than or equal to 75%, greater than or equal to 80%, greater than or equal to 85%, greater than or equal to 90%, or greater than or equal to 95% intact RNA (e.g., when administered to a subject, at the time of expiration, after storage, and/or at the end of its shelf-life). In certain instances, the total amount of RNA (e.g., mRNA) comprises less than or equal to 95%, less than or equal to 90%, less than or equal to 85%, less than or equal to 80%, less than or equal to 75%, less than or equal to 70%, less than or equal to 65%, less than or equal to 60%, less than or equal to 55%, or less than or equal to 50% intact RNA (e.g., when administered to a subject, at the time of expiration, after storage, and/or at the end of its shelf-life). Combinations of these ranges are also possible (e.g., greater than or equal to 40% and less than or equal to 95%, greater than or equal to 40% and less than or equal to 80%, greater than or equal to 40% and less than or equal to 70%, greater than or equal to 70% and less than or equal to 95%, greater than or equal to 75% and less than or equal to 90%, or greater than or equal to 75% and less than or equal to 80%).

In certain cases, the percentage of intact RNA (e.g., mRNA) (e.g., in the container) comprises the percentage of intact RNA that would degrade in the liquid pharmaceutical composition over a shelf-life of the article+greater than or equal to 15%, greater than or equal to 20%, greater than or equal to 25%, greater than or equal to 30%, greater than or equal to 35%, greater than or equal to 40%, greater than or equal to 45%, greater than or equal to 50%, greater than or equal to 55%, greater than or equal to 60%, greater than or equal to 65%, greater than or equal to 70%, or greater than or equal to 75% of the total RNA. In some instances, the percentage of intact RNA (e.g., mRNA) (e.g., in the container) comprises the percentage of intact RNA that would degrade in the liquid pharmaceutical composition over a shelf-life of the article+less than or equal to 80%, less than or equal to 75%, less than or equal to 70%, less than or equal to 65%, less than or equal to 60%, less than or equal to 55%, less than or equal to 50%, less than or equal to 45%, or less than or equal to 40% of the total RNA. Combinations of these ranges are also possible (e.g., the percentage of intact RNA that would degrade in the liquid pharmaceutical composition over a shelf-life of the article+greater than or equal to 15% and less than or equal to 80% of the total RNA, the percentage of intact RNA that would degrade in the liquid pharmaceutical composition over a shelf-life of the article+greater than or equal to 25% and less than or equal to 70%, or the percentage of intact RNA that would degrade in the liquid pharmaceutical composition over a shelf-life of the article+greater than or equal to 40% and less than or equal to 60%).

In some embodiments, the total amount of RNA (e.g., mRNA) comprises greater than or equal to 5%, greater than or equal to 10%, greater than or equal to 15%, greater than or equal to 20%, greater than or equal to 25%, greater than or equal to 30%, greater than or equal to 35%, greater than or equal to 40%, greater than or equal to 45%, greater than or equal to 50%, or greater than or equal to 55% RNA that is less than full length RNA (e.g., fragmented RNA) (e.g., when administered to a subject, at the time of expiration, after storage, and/or at the end of its shelf-life). In certain embodiments, the total amount of RNA (e.g., mRNA) comprises less than or equal to 60%, less than or equal to 55%, less than or equal to 50%, less than or equal to 45%, less than or equal to 40%, less than or equal to 35%, less than or equal to 30%, less than or equal to 25%, less than or equal to 20%, less than or equal to 15%, or less than or equal to 10% RNA that is less than full length RNA (e.g., fragmented RNA) (e.g., when administered to a subject, at the time of expiration, after storage, and/or at the end of its shelf-life). Combinations of these ranges are also possible (e.g., greater than or equal to 5% and less than or equal to 60%, greater than or equal to 20% and less than or equal to 60%, greater than or equal to 30% and less than or equal to 60%, greater than or equal to 5% and less than or equal to 30%, or greater than or equal to 20% and less than or equal to 25%).

According to certain embodiments, the total amount of RNA (e.g., mRNA) in the container has a value of at least the number of individual doses in the container times 5% greater (e.g., at least 10% greater, 15% greater, 20% greater, 25% greater, 30% greater, 35% greater, 40% greater, 45% greater, or 50% greater) than the amount of the effective dose of RNA within each individual dose. In some embodiments, the total amount of RNA (e.g., mRNA) in the container has a value of less than or equal to the number of individual doses in the container times 100% greater (e.g., less than or equal to 80% greater, 60% greater, 50% greater, 40% greater, 30% greater, 25% greater, 20% greater, or 10% greater) than the amount of the effective dose of RNA within each individual dose. Combinations of these ranges are also possible (e.g., at least the number of individual doses in the container times 5% greater than the amount of the effective dose of RNA within each individual dose and less than or equal to the number of individual doses in the container times 100% greater than the amount of the effective dose of RNA within each individual dose, at least the number of individual doses in the container times 20% greater than the amount of the effective dose of RNA within each individual dose and less than or equal to the number of individual doses in the container times 50% greater than the amount of the effective dose of RNA within each individual dose). For example, if the total amount of RNA in the container has a value of at least the number of individual doses in the container times 5% greater than the amount of the effective dose of RNA within each individual dose, the container has 10 individual doses, and each dose is 100 micrograms of RNA, then the container would have at least (1.05*10 * 100) 1,050 micrograms.

In some embodiments, an individual dose is the individual dose needed to produce a therapeutically effective amount of a protein in the subject. In certain instances, the individual dose of the liquid pharmaceutical composition is the individual dose of the liquid pharmaceutical composition needed at the time of manufacturing to produce a therapeutically effective amount of a protein in the subject. In certain cases, an individual dose is the individual dose approved by a regulatory agency (such as the FDA) to stimulate an antigen specific immune response in the subject.

In certain embodiments, an effective dose and/or effective amount of RNA (e.g., mRNA) (e.g., intact RNA) is the amount of RNA (e.g., mRNA) (e.g., intact RNA) needed to produce a therapeutically effective amount of a protein in the subject. In certain cases, an effective dose and/or effective amount of RNA (e.g., mRNA) (e.g., intact RNA) is the amount of RNA (e.g., mRNA) (e.g., intact RNA) approved by a regulatory agency (such as the FDA) to stimulate an antigen specific immune response in the subject.

As used herein, the term “amount” refers to total mass (e.g., mg). As a person of ordinary skill in the art would understand, the total mass of a component (e.g., RNA) may be adjusted in multiple ways. For example, if an article is comprised of a solution comprising RNA, the total mass of the RNA in the article could be increased in multiple ways, such as adding more of the RNA to the article (e.g., by increasing the concentration of the RNA in the solution) and/or increasing the volume of the solution (e.g., a solution with a constant concentration). Thus, the amount of a liquid pharmaceutical composition is an amount comprising a total mass of RNA. An amount of RNA is a mass of RNA. An amount of intact RNA is a mass of full length RNA.

Similarly, as used herein, the term “dose” or “individual dose” refers to total mass (e.g., mg). For example, a dose of full length RNA is 50 mg of full length RNA in some embodiments. As a person of ordinary skill in the art would understand, while a dose may be referred to in units other than mass (e.g., 1 pill, 2 capsules, 1 tube of ointment, 2 drops, 1 mL of solution, etc.), the dose may always be translated into mass. For example, if a dose is 1 mL of a liquid pharmaceutical composition, and the liquid pharmaceutical composition has a density of 10 mg/mL, and the concentration of full length RNA in the liquid pharmaceutical is 1 mg/mL, then the dose of liquid pharmaceutical composition is 10 mg of liquid pharmaceutical composition and the dose of full length RNA is 1 mg. A baseline dose is a dose having a specific mass of RNA prior to storage of a composition.

In certain embodiments, an individual dose and/or effective amount is at least 5 micrograms, at least 10 micrograms, at least 20 micrograms, at least 30 micrograms, at least 40 micrograms, at least 50 micrograms, at least 60 micrograms, at least 70 micrograms, at least 80 micrograms, at least 90 micrograms, at least 100 micrograms, at least 125 micrograms, or at least 150 micrograms of intact mRNA. In some embodiments, an individual dose and/or effective amount is less than or equal to 200 micrograms, less than or equal to 175 micrograms, less than or equal to 150 micrograms, less than or equal to 125 micrograms, less than or equal to 100 micrograms, less than or equal to 90 micrograms, less than or equal to 80 micrograms, less than or equal to 70 micrograms, less than or equal to 60 micrograms, less than or equal to 50 micrograms, or less than or equal to 40 micrograms. Combinations of these ranges are also possible (e.g., at least 5 micrograms and less than or equal to 200 micrograms, at least 20 micrograms and less than or equal to 50 micrograms, or at least 40 micrograms and less than or equal to 60 micrograms).

In some embodiments, a composition and/or an article (e.g., a container) comprises at least 5%, at least 10%, at least 15%, at least 20%, at least 25%, at least 30%, at least 40%, at least 50%, at least 60%, at least 70%, at least 80%, at least 90% more intact RNA than an individual dose and/or effective amount of the intact RNA. In certain embodiments, a composition and/or an article (e.g., a container) comprises less than or equal to 200%, less than or equal to 150%, less than or equal to 100%, less than or equal to 90%, less than or equal to 80%, less than or equal to 70%, less than or equal to 60%, less than or equal to 50%, less than or equal to 40%, less than or equal to 30%, or less than or equal to 20% more intact RNA than an individual dose and/or effective amount of the intact RNA. Combinations of these ranges are also possible (e.g., at least 5% and less than or equal to 20, at least 20% and less than or equal to 100%, or at least 20% and less than or equal to 50%).

In some embodiments, the article has a particular shelf-life at a particular temperature. As used herein, the shelf-life is the amount of time for which the article can be stored in a particular set of conditions and still be used safely and effectively (e.g., the amount of time for which the article can be stored in a particular set of conditions and still be used according to FDA guidelines). For example, in certain cases, the article has a shelf-life of and/or can be stored (or is stored) for greater than or equal to 1 month, greater than or equal to 2 months, greater than or equal to 3 months, greater than or equal to 6 months, or greater than or equal to 9 months. In some embodiments, the article has a shelf-life of and/or can be stored (or is stored) for less than or equal to 1 year, less than or equal to 9 months, or less than or equal to 6 months. Combinations of these ranges are also possible (e.g., greater than or equal to 3 months and less than or equal to 1 year).

In some instances, the shelf-life is determined when stored at a temperature of (and/or the composition and/or article can be stored (or is stored) at a temperature of) greater than 0° C., greater than or equal to 1° C., greater than or equal to 2° C., greater than or equal to 3° C., greater than or equal to 4° C., or greater than or equal to 5° C. In certain embodiments, the shelf-life is determined when stored at a temperature of (and/or the composition and/or article can be stored (or is stored) at a temperature of) less than or equal to 10° C., less than or equal to 9° C., less than or equal to 8° C., less than or equal to 7° C., less than or equal to 6° C., or less than or equal to 5° C. Combinations of these ranges are also possible (e.g., greater than 0° C. and less than or equal to 10° C., or 5° C.).

As used herein, the shelf-life is determined at standard pressure and in the absence of any additional components (e.g., contaminations or stabilizers) that do not form part of the article and/or liquid pharmaceutical composition (e.g., do not form part of the article and/or liquid pharmaceutical composition as approved by the FDA).

In some embodiments, the shelf-life comprises a first period of time at a first temperature followed by a second period of time at a second temperature. In some instances, the first period of time is greater than the second period of time. In certain embodiments, the second temperature is higher than the first temperature. For example, in some cases, the article (e.g., liquid pharmaceutical composition) may be stored frozen (e.g., at −70° C.) for a period of time (such as greater than or equal to 1 year after it is filled). In some embodiments the first period of time can be at multiple frozen temperatures (e.g., −70° C. and then −20° C.). In some cases, it may then be transported to a consumer, where it may be stored as a liquid (e.g., at 5° C.) for greater than or equal to 3 months.

In certain cases, the first period of time is greater than or equal to 3 months, greater than or equal to 6 months, greater than or equal to 9 months, greater than or equal to 1 year, greater than or equal to 15 months, or greater than or equal to 18 months. In some instances, the first period of time is less than or equal to 2 years, less than or equal to 18 months, less than or equal to 1 year, or less than or equal to 6 months. Combinations of these range are also possible (e.g., greater than or equal to 3 months and less than or equal to 2 years).

In some instances, the first temperature is less than or equal to −20° C., less than or equal to −30° C., less than or equal to −40° C., less than or equal to −50° C., less than or equal to −60° C., or less than or equal to −70° C. In certain embodiments, the first temperature is greater than or equal to −90° C., greater than or equal to −80° C., greater than or equal to −70° C., greater than or equal to −60° C., greater than or equal to −50° C., greater than or equal to −40° C., or greater than or equal to −30° C. Combinations of these ranges are also possible (e.g., less than or equal to −20° C. and greater than or equal to −90° C., less than or equal to −50° C. and greater than or equal to −90° C., or −70° C.).

In certain embodiments, the second period is greater than or equal to 1 month, greater than or equal to 2 months, greater than or equal to 3 months, greater than or equal to 6 months, or greater than or equal to 9 months. In some embodiments, the second period is less than or equal to 1 year, less than or equal to 9 months, or less than or equal to 6 months. Combinations of these ranges are also possible (e.g., greater than or equal to 3 months and less than or equal to 1 year).

In some embodiments, the second temperature is greater than 0° C., greater than or equal to 1° C., greater than or equal to 2° C., greater than or equal to 3° C., greater than or equal to 4° C., or greater than or equal to 5° C. In certain embodiments, the second temperature is less than or equal to 10° C., less than or equal to 9° C., less than or equal to 8° C., less than or equal to 7° C., less than or equal to 6° C., or less than or equal to 5° C. Combinations of these ranges are also possible (e.g., greater than 0° C. and less than or equal to 10° C., or 5° C.).

In certain embodiments, a particular percentage of the RNA (e.g., mRNA) is intact at the end of the shelf-life and/or after storage (e.g., after 3 months at 5° C.). For example, in certain cases, greater than or equal to 15%, greater than or equal to 18%, greater than or equal to 20%, greater than or equal to 25%, greater than or equal to 30%, greater than or equal to 35%, greater than or equal to 40%, greater than or equal to 45%, greater than or equal to 50%, greater than or equal to 55%, greater than or equal to 60%, greater than or equal to 65%, greater than or equal to 70%, greater than or equal to 75%, greater than or equal to 80%, greater than or equal to 85%, greater than or equal to 90%, or greater than or equal to 95% of the RNA (e.g., mRNA) is intact at the end of the shelf-life and/or after storage. In some instances, less than or equal to 95%, less than or equal to 90%, less than or equal to 85%, less than or equal to 80%, less than or equal to 75%, less than or equal to 70%, less than or equal to 65%, less than or equal to 60%, less than or equal to 55%, less than or equal to 50%, less than or equal to 45%, less than or equal to 40%, less than or equal to 35%, less than or equal to 30%, less than or equal to 25%, or less than or equal to 20% of the RNA (e.g., mRNA) is intact at the end of the shelf-life and/or after storage. Combinations of these ranges are also possible (e.g., greater than or equal to 15% and less than or equal to 95%, greater than or equal to 40% and less than or equal to 95%, greater than or equal to 40% and less than or equal to 80%, greater than or equal to 40% and less than or equal to 70%, greater than or equal to 70% and less than or equal to 95%, greater than or equal to 75% and less than or equal to 90%, or greater than or equal to 75% and less than or equal to 80%).

In some embodiments, methods of filling an article (e.g., any article described herein) are described. In certain embodiments, the method comprises adding a nucleic acid (e.g., RNA, such as mRNA) to the article. In some cases, the method comprises adding a lipid carrier (e.g., a lipid nanoparticle, liposome, and/or lipoplex) to the article. In certain instances, the nucleic acid (e.g., mRNA) and lipid carrier (e.g., LNP) may be added separately or in combination (e.g., in the form of a liquid pharmaceutical composition, for example, where the nucleic acid (e.g., mRNA) is formulated in the lipid carrier (e.g., LNP)). In some embodiments, the method comprises freezing the nucleic acid (e.g., mRNA) and/or lipid carrier (e.g., LNP) (individually or in combination as a pharmaceutical composition) prior to addition to the article. According to some embodiments, the addition of the nucleic acid (e.g., mRNA) and/or the lipid carrier (or the liquid pharmaceutical composition) forms an amount of a liquid pharmaceutical composition in the article.

According to some embodiments, the amount of the liquid pharmaceutical composition formed in the article is greater than or equal to (1+the fraction of the RNA that would degrade in the liquid pharmaceutical composition over the shelf-life of the article)×(an individual dose of the liquid pharmaceutical composition). In some embodiments, the amount is greater than or equal to 1.05×(an individual dose of the liquid pharmaceutical composition), greater than or equal to 1.07×(an individual dose of the liquid pharmaceutical composition), greater than or equal to 1.08×(an individual dose of the liquid pharmaceutical composition), greater than or equal to 1.10×(an individual dose of the liquid pharmaceutical composition), greater than or equal to 1.15×(an individual dose of the liquid pharmaceutical composition), greater than or equal to 1.2×(an individual dose of the liquid pharmaceutical composition), greater than or equal to 1.25×(an individual dose of the liquid pharmaceutical composition), or greater than or equal to 1.3×(an individual dose of the liquid pharmaceutical composition). In certain embodiments, the amount is less than or equal to 2.00×(an individual dose of the liquid pharmaceutical composition), less than or equal to 1.8×(an individual dose of the liquid pharmaceutical composition), less than or equal to 1.6×(an individual dose of the liquid pharmaceutical composition), less than or equal to 1.4×(an individual dose of the liquid pharmaceutical composition), less than or equal to 1.3×(an individual dose of the liquid pharmaceutical composition), less than or equal to 1.25×(an individual dose of the liquid pharmaceutical composition), less than or equal to 1.2×(an individual dose of the liquid pharmaceutical composition), or less than or equal to 1.1×(an individual dose of the liquid pharmaceutical composition). Combinations of these ranges are also possible (e.g., greater than or equal to 1.05×(an individual dose of the liquid pharmaceutical composition) and less than or equal to 2.00×(an individual dose of the liquid pharmaceutical composition) or greater than or equal to 1.2×(an individual dose of the liquid pharmaceutical composition) and less than or equal to 1.3×(an individual dose of the liquid pharmaceutical composition)).

In accordance with certain embodiments, the method comprises storing the article for a duration of time (e.g., up to 1 year or up to 3 years) at a temperature (e.g., greater than 0° C. and less than 10° C., or 5° C.). In some instances, the method comprises storing the article for a duration of time up to the shelf-life of the article (e.g., any shelf-life described herein).

In certain cases, a particular percentage of the RNA (e.g., mRNA) is intact after the storing step (e.g., a particular percentage of the RNA is intact if stored for the shelf-life of the article). For example, in some instances, greater than or equal to 15%, greater than or equal to 18%, greater than or equal to 20%, greater than or equal to 25%, greater than or equal to 30%, greater than or equal to 35%, greater than or equal to 40%, greater than or equal to 45%, greater than or equal to 50%, greater than or equal to 55%, greater than or equal to 60%, greater than or equal to 65%, greater than or equal to 70%, greater than or equal to 75%, greater than or equal to 80%, greater than or equal to 85%, greater than or equal to 90%, or greater than or equal to 95% of the RNA (e.g., mRNA) is intact after the storing step. In some instances, less than or equal to 95%, less than or equal to 90%, less than or equal to 85%, less than or equal to 80%, less than or equal to 75%, less than or equal to 70%, less than or equal to 65%, less than or equal to 60%, less than or equal to 55%, less than or equal to 50%, less than or equal to 45%, less than or equal to 40%, less than or equal to 35%, less than or equal to 30%, less than or equal to 25%, or less than or equal to 20% of the RNA (e.g., mRNA) is intact after the storing step. Combinations of these ranges are also possible (e.g., greater than or equal to 15% and less than or equal to 95%, greater than or equal to 40% and less than or equal to 95%, greater than or equal to 40% and less than or equal to 80%, greater than or equal to 40% and less than or equal to 70%, greater than or equal to 70% and less than or equal to 95%, greater than or equal to 75% and less than or equal to 90%, or greater than or equal to 75% and less than or equal to 80%).

In some embodiments, the percentage of the RNA (e.g., mRNA) that is intact after the storing step is lower than the percentage of the RNA (e.g., mRNA) that is intact prior to the storing step. In certain embodiments, the percentage of the RNA (e.g., mRNA) that is intact prior to the storing step is at least 40%, such as at least 50%, at least 55%, at least 60%, at least 63%, at least 65%, at least 70%, at least 75%, at least 80%, at least 85%, at least 90%, or at least 95%. In some cases, the percentage of the RNA (e.g., mRNA) that is intact prior to the storing step is less than or equal to 100%, less than or equal to 99%, less than or equal to 98%, less than or equal to 95%, less than or equal to 90%, less than or equal to 85%, less than or equal to 80%, less than or equal to 75%, less than or equal to 70%, less than or equal to 65%, less than or equal to 3%, less than or equal to 60%, less than or equal to 55%, or less than or equal to 55%. Combinations of these ranges are also possible (e.g., at least 40% and less than or equal to 100%, at least 40% and less than or equal to 90%, or at least 50% and less than or equal to 80%).

In certain embodiments, the total amount of intact RNA (e.g., mRNA) prior to storage and/or the total amount of intact RNA (e.g., mRNA) after storage is greater than or equal to an effective amount of intact RNA.

In some instances, the storing step does not include storing at the glass transition temperature of the composition (e.g., liquid pharmaceutical composition). In certain embodiments, the storing step does not include storing at a temperature of greater than or equal to −50° C., greater than or equal to −45° C., greater than or equal to −40° C., or greater than or equal to −35° C. In certain cases, the storing step does not include storing at a temperature of less than or equal to −20° C., less than or equal to −25° C., less than or equal to −30° C., less than or equal to −35° C., or less than or equal to −40° C. Combinations of these ranges are also possible (e.g., greater than or equal to −50° C. and less than or equal to −20° C., greater than or equal to −45° C. and less than or equal to −30° C., or greater than or equal to −35° C. and less than or equal to −30° C.).

In certain embodiments, the method (e.g., any method disclosed herein) and/or composition and/or article (e.g., any article disclosed herein) mitigates and/or accounts for degradation (e.g., from transesterification) of RNA (e.g., mRNA, such as any mRNA disclosed herein). For example, in some embodiments, the method and/or composition and/or article mitigates and/or accounts for degradation of RNA at certain conditions (e.g., any conditions disclosed herein, such as the shelf-life conditions and/or storage conditions disclosed herein, such as in a refrigerator, such as at 5° C.). In some cases, the method and/or composition and/or article mitigates and/or accounts for degradation of RNA (e.g., at certain conditions) by ensuring that a sufficient amount of intact RNA is provided at the time of administration and/or throughout the shelf-life of the article. In certain instances, ensuring that a sufficient amount of intact RNA is provided at the time of administration and/or throughout the shelf-life of the article comprises providing a sufficient amount of intact RNA at the time of manufacture and/or sale (e.g., providing a sufficient amount of intact RNA at the time of manufacture and/or sale taking into account the amount of RNA that will degrade until the time of administration and/or throughout the shelf-life). In some embodiments, the total amount of intact RNA prior to storage of the composition for a period of time (e.g., as disclosed elsewhere herein) is calculated to account for degradation of the mRNA (e.g., from transesterification of the mRNA) during the storage of the composition for the period of time and/or to ensure at least an effective amount of intact RNA is present throughout the storage and/or shelf-life (and/or at the time of administration).

In some embodiments, methods of delivering an effective dose of a nucleic acid (e.g., RNA, such as mRNA) are described herein. In certain embodiments, the method comprises administering a liquid pharmaceutical composition (e.g., any composition or liquid pharmaceutical composition disclosed herein) to a subject. For example, in accordance with certain embodiments, the liquid pharmaceutical composition comprises a nucleic acid (e.g., any nucleic acid disclosed herein, such as an RNA or mRNA encoding a protein) and a lipid carrier (e.g., any lipid carrier disclosed herein, such as an LNP).

In some cases, a total dose of nucleic acid (e.g., RNA, such as mRNA) is administered to the subject that is at least 5% (e.g., at least 10%, at least 15%, at least 20%, at least 25%, at least 30%, at least 35%, at least 40%, at least 45%, at least 50%; less than or equal to 100%, less than or equal to 80%, less than or equal to 60%, less than or equal to 50%, less than or equal to 40%, less than or equal to 30%, less than or equal to 25%, less than or equal to 20%, less than or equal to 15%, or less than or equal to 10%; combinations of these ranges are also possible (e.g., at least 5% and less than or equal to 100% or at least 20% and less than or equal to 50%) greater than an effective dose of the nucleic acid (e.g., mRNA).

In some embodiments, a subject to which a composition comprising a nucleic acid (e.g., mRNA) formulated in a lipid (e.g., LNP) is administered is a subject that suffers from or is at risk of suffering from a disease, disorder or condition, including a communicable or non-communicable disease, disorder or condition. As used herein, “treating” a subject can include either therapeutic use or prophylactic use relating to a disease, disorder or condition, and may be used to describe uses for the alleviation of symptoms of a disease, disorder or condition, uses for vaccination against a disease, disorder or condition, and uses for decreasing the contagiousness of a disease, disorder or condition, among other uses.

In certain embodiments, the nucleic acid (e.g., RNA, such as mRNA) encodes a therapeutic protein. In some embodiments the nucleic acid is an mRNA vaccine designed to achieve particular biologic effects. Exemplary vaccines of the invention feature mRNAs encoding a particular antigen of interest (or an mRNA or mRNAs encoding antigens of interest). In exemplary aspects, the vaccines of the invention feature an mRNA or mRNAs encoding antigen(s) derived from infectious diseases or cancers.

Diseases or conditions, in some embodiments include those caused by or associated with infectious agents, such as bacteria, viruses, fungi and parasites. Non-limiting examples of such infectious agents include Gram-negative bacteria, Gram-positive bacteria, RNA viruses (including (+)ssRNA viruses, (−)ssRNA viruses, dsRNA viruses), DNA viruses (including dsDNA viruses and ssDNA viruses), reverse transcriptase viruses (including ssRNA-RT viruses and dsDNA-RT viruses), protozoa, helminths, and ectoparasites.

In certain embodiments, the article comprises a vaccine (e.g., an infectious disease vaccine). In some embodiments, the antigen comprises an infectious disease antigen. The antigen of the infectious disease vaccine is a viral or bacterial antigen. In some embodiments the infectious agent is a strain of virus selected from the group consisting of adenovirus; Herpes simplex, type 1; Herpes simplex, type 2; encephalitis virus, papillomavirus, Varicella-zoster virus; Epstein-barr virus; Human cytomegalovirus; Human herpes virus, type 8; Human papillomavirus; BK virus; JC virus; Smallpox; polio virus; Hepatitis B virus; Human bocavirus; Parvovirus B19; Human astrovirus; Norwalk virus; coxsackievirus; hepatitis A virus; poliovirus; rhinovirus; Severe acute respiratory syndrome virus; Hepatitis C virus; Yellow Fever virus; Dengue virus; West Nile virus; Rubella virus; Hepatitis E virus; Human Immunodeficiency virus (HIV); Influenza virus; Guanarito virus; Junin virus; Lassa virus; Machupo virus; Sabia virus; Crimean-Congo hemorrhagic fever virus; Ebola virus; Marburg virus; Measles virus; Mumps virus; Parainfluenza virus; Respiratory syncytial virus; Human metapneumovirus; Hendra virus; Nipah virus; Rabies virus; Hepatitis D; Rotavirus; Orbivirus; Coltivirus; Banna virus; Human Enterovirus; Japanese encephalitis virus; Vesicular exanthernavirus; and Eastern equine encephalitis.

In some embodiments, a disease, disorder or condition is caused by or associated with a virus.

In some embodiments, a disease, disorder or condition is caused by or associated with a Plasmodium parasite. In some embodiments, the disease, disorder or condition is malaria. In some embodiments, the Plasmodium parasite is P. falciparum, P. malariae, P. ovale, P. vivax or P. knowlesi.

In some embodiments, a disease, disorder or condition is caused by or associated with a malignant cell. In some embodiments, the disease, disorder or condition is cancer. In some embodiments, the cancer is acute lymphoblastic leukemia (ALL), acute myeloid leukemia (AML), adrenocortical carcinoma, AIDS-related cancer (including Kaposi sarcoma, AIDS-related lymphoma and primary CNS lymphoma), anal cancer, appendix cancer, astrocytoma, atypical reratoid/rhabdoid tumor, basal cell carcinoma of the skin, bile duct cancer, bladder cancer, bone cancer (including Ewing sarcoma, osteosarcoma and malignant fibrous histiocytoma), brain cancer, breast cancer, Burkitt lymphoma, cancer of the central nervous system (including medulloblastoma, germ cell tumor and primary CNS lymphoma), cervical cancer, cholangiocarcinoma, chordoma, chronic lymphocytic leukemia (CLL), chronic myelogenous leukemia (CML), chronic myeloproliferative neoplasm, colorectal cancer, craniopharyngioma, cutaneous T-cell lymphoma, ductal carcinoma in situ (DCIS), endometrial cancer (uterine cancer), ependymoma, esophageal cancer, esthesioneuroblastoma, extracranial germ cell tumor, extragonadal germ cell tumor, eye cancer (including intraocular melanoma, uveal melanoma and retinoblastoma), Fallopian tube cancer, gallbladder cancer, gastric (stomach) cancer, gastrointestinal carcinoid tumor, gastrointestinal stromal tumors (GIST), germ cell tumors (including childhood central nervous system germ cell tumors, childhood extracranial germ cell tumors, extragonadal germ cell tumors, ovarian germ cell tumors and testicular cancer), gestational trophoblastic disease, hairy cell leukemia, head and neck cancer, childhood heart tumors, hepatocellular cancer, Langerhans cell histiocytosis, Hodgkin lymphoma, hypopharyngeal cancer, islet cell tumors, pancreatic neuroendocrine tumors, Kaposi sarcoma, kidney (renal cell) cancer, laryngeal cancer, leukemia, lip and oral cavity cancer, liver cancer, lung cancer (including non-small cell lung cancer, small cell lung cancer, pleuropulmonary blastoma, and tracheobronchial tumor), lymphoma, melanoma, Merkel cell carcinoma, mesothelioma, midline tract carcinoma with NUT gene changes, mouth cancer, multiple endocrine neoplasia syndromes, multiple myeloma/plasma cell neoplasms, mycosis fungoides, myelodysplastic syndromes, nasal cavity and paranasal sinus cancer, nasopharyngeal cancer, neuroblastoma, non-Hodgkin lymphoma, oral cancer, oropharyngeal cancer, ovarian cancer, pancreatic cancer, pancreatic neuroendocrine tumors (islet cell tumors), paraganglioma, paranasal sinus and nasal cavity cancer, parathyroid cancer, penile cancer, pharyngeal cancer, pheochromocytoma, pituitary tumor, plasma cell neoplasm/multiple myeloma, primary peritoneal cancer, prostate cancer, rectal cancer, rhabdomyosarcoma, salivary gland cancer, sarcoma (including rhabdomyosarcoma, childhood vascular tumors, Ewing sarcoma, Kaposi sarcoma, osteosarcoma, soft tissue sarcoma and uterine sarcoma), Sézary syndrome, skin cancer, small intestine cancer, squamous cell carcinoma of the skin, testicular cancer, throat cancer (including nasopharyngeal cancer, oropharyngeal cancer and hypopharyngeal cancer), thymoma and thymic carcinoma, thyroid cancer, tracheobronchial tumors, transitional cell cancer of the renal pelvis and ureter, urethral cancer, vaginal cancer, vulvar cancer, or Wilms tumor.

The vaccines may be traditional or personalized cancer or infectious disease vaccines. A traditional cancer vaccine, for instance, is a vaccine including a cancer antigen that is known to be found in cancers or tumors generally or in a specific type of cancer or tumor. Antigens that are expressed in or by tumor cells are referred to as “tumor associated antigens”. A particular tumor associated antigen may or may not also be expressed in non-cancerous cells. Many tumor mutations are known in the art. Personalized vaccines, for instance, may include RNA (e.g., mRNA) encoding for one or more known cancer antigens specific for the tumor or cancer antigens specific for each subject (e.g., personalized cancer antigen), referred to as neoepitopes or patient specific epitopes or antigens. A “patient specific cancer antigen” is an antigen that has been identified as being expressed in a tumor of a particular patient. The patient specific cancer antigen may or may not be typically present in tumor samples generally. Tumor associated antigens that are not expressed or rarely expressed in non-cancerous cells, or whose expression in non-cancerous cells is sufficiently reduced in comparison to that in cancerous cells and that induce an immune response induced upon vaccination, are referred to as neoepitopes.

The compositions of the invention are also useful for treating or preventing a symptom of diseases characterized by missing or aberrant protein activity, by replacing the missing protein activity or overcoming the aberrant protein activity. Because of the rapid initiation of protein production following introduction of mRNAs, as compared to viral DNA vectors, the compounds of the present disclosure are particularly advantageous in treating acute diseases such as sepsis, stroke, and myocardial infarction. Moreover, the lack of transcriptional regulation of the alternative mRNAs of the present disclosure is advantageous in that accurate titration of protein production is achievable. Multiple diseases are characterized by missing (or substantially diminished such that proper protein function does not occur) protein activity. Such proteins may not be present, are present in very low quantities or are essentially non-functional. The present disclosure provides a method for treating such conditions or diseases in a subject by introducing polynucleotide or cell-based therapeutics containing the alternative polynucleotides provided herein, wherein the alternative polynucleotides encode for a protein that replaces the protein activity missing from the target cells of the subject.

Diseases characterized by dysfunctional or aberrant protein activity include, but are not limited to, cancer and other proliferative diseases, genetic diseases (e.g., cystic fibrosis), autoimmune diseases, diabetes, neurodegenerative diseases, cardiovascular diseases, and metabolic diseases. The present disclosure provides a method for treating such conditions or diseases in a subject by introducing polynucleotide or cell-based therapeutics containing the polynucleotides provided herein, wherein the polynucleotides encode for a protein that antagonizes or otherwise overcomes the aberrant protein activity present in the cell of the subject.

Specific examples of a dysfunctional protein are the missense or nonsense mutation variants of the cystic fibrosis transmembrane conductance regulator (CFTR) gene, which produce a dysfunctional or nonfunctional, respectively, protein variant of CFTR protein, which causes cystic fibrosis.

Thus, provided are methods of treating cystic fibrosis in a mammalian subject by contacting a cell of the subject with an alternative polynucleotide having a translatable region that encodes a functional CFTR polypeptide, under conditions such that an effective amount of the CTFR polypeptide is present in the cell. Preferred target cells are epithelial cells, such as the lung, and methods of administration are determined in view of the target tissue; i.e., for lung delivery, the polynucleotides are formulated for administration by inhalation.

In another embodiment, the present disclosure provides a method for treating hyperlipidemia in a subject, by introducing into a cell population of the subject with a polynucleotide molecule encoding Sortilin, thereby ameliorating the hyperlipidemia in a subject. The SORT1 gene encodes a trans-Golgi network (TGN) transmembrane protein called Sortilin.

In certain embodiments, the polypeptide of interest encoded by the polynucleotide of the invention is granulocyte colony-stimulating factor (GCSF), and the polynucleotide or pharmaceutical composition of the invention is for use in treating a neurological disease such as cerebral ischemia, or treating neutropenia, or for use in increasing the number of hematopoietic stem cells in the blood (e.g., before collection by leukapheresis for use in hematopoietic stem cell transplantation).

In certain embodiments, the polypeptide of interest encoded by the polynucleotide of the invention is erythropoietin (EPO), and the polynucleotide or pharmaceutical composition of the invention is for use in treating anemia, inflammatory bowel disease (such as Crohn's disease and/or ulcer colitis), or myelodysplasia.

In some embodiments, “administering” or “administration” means providing a material to a subject in a manner that is pharmacologically useful. In some embodiments, a composition disclosed herein is administered to a subject enterally. In some embodiments, an enteral administration of the composition is oral. In some embodiments, a composition disclosed herein is administered to the subject parenterally. In some embodiments, a composition disclosed herein is administered to a subject subcutaneously, intraocularly, intravitreally, subretinally, intravenously (IV), intracerebro-ventricularly, intramuscularly, intrathecally (IT), intracisternally, intraperitoneally, via inhalation, topically, or by direct injection to one or more cells, tissues, or organs.

To “treat” a disease as the term is used herein, means to reduce the frequency or severity of at least one sign or symptom of a disease, disorder or condition experienced by a subject. The compositions described above or elsewhere herein are typically administered to a subject in an effective amount, that is, an amount capable of producing a desirable result. The desirable result will depend upon the active agent being administered. For example, an effective amount of a composition comprising a nucleic acid (e.g., mRNA) formulated in a lipid (e.g., LNP) may be an amount of the composition that is capable of increasing expression of a protein in the subject. A therapeutically acceptable amount may be an amount that is capable of treating a disease or condition, e.g., a disease or condition that that can be relieved by increasing expression of a protein in a subject. As is well known in the medical and veterinary arts, dosage for any one subject depends on many factors, including the subject's size, body surface area, age, the particular composition to be administered, the active ingredient(s) in the composition, the intended outcome of the administration, time and route of administration, general health, and other drugs being administered concurrently.

In some embodiments, a subject is administered a composition comprising a nucleic acid (e.g., mRNA) formulated in a lipid (e.g., LNP) in an amount sufficient to increase expression of a protein in the subject.

In certain embodiments, LNP preparations (e.g., populations or formulations) are analyzed for polydispersity in size (e.g., particle diameter) and/or composition (e.g., amino lipid amount or concentration, phospholipid amount or concentration, structural lipid amount or concentration, PEG-lipid amount or concentration, mRNA amount (e.g., mass) or concentration) and, optionally, further assayed for in vitro and/or in vivo activity. Fractions or pools thereof can also be analyzed for accessible mRNA and/or purity (e.g., purity as determined by reverse-phase (RP) chromatography).

Particle size (e.g., particle diameter) can be determined by Dynamic Light Scattering (DLS). DLS measures a hydrodynamic diameter. Smaller particles diffuse more quickly, leading to faster fluctuations in the scattering intensity and shorter decay times for the autocorrelation function. Larger particles diffuse more slowly, leading to slower fluctuations in the scattering intensity and longer decay times in the autocorrelation function.

mRNA purity can be determined by high-performance liquid chromatography (HPLC) (e.g., reverse phase high-performance liquid chromatography (RP-HPLC) or reverse phase high-performance liquid chromatography (RP-HPLC) size based separation) or capillary electrophoresis (CE) (e.g., frontal analysis capillary electrophoresis (FA-CE)). Reverse phase high-performance liquid chromatography (RP-HPLC) size based separation can be used to assess mRNA integrity by a length-based gradient RP separation and UV detection of RNA at 260 nm. As used herein “main peak” or “main peak purity” refers to the RP-HPLC signal detected from mRNA that corresponds to the full size mRNA molecule loaded within a given LNP formulation. mRNA purity can also be assessed by fragmentation analysis. Fragmentation analysis (FA) is a method by which nucleic acid (e.g., mRNA) fragments can be analyzed by capillary electrophoresis. Fragmentation analysis involves sizing and quantifying nucleic acids (e.g., mRNA), for example by using an intercalating dye coupled with an LED light source. Such analysis may be completed, for example, with a Fragment Analyzer from Advanced Analytical Technologies, Inc.

Compositions formed via the methods described herein may be particularly useful for administering an agent to a subject in need thereof. In some embodiments, the compositions are used to deliver a pharmaceutically active agent. In some instances, the compositions are used to deliver a prophylactic agent. The compositions may be administered in any way known in the art of drug delivery, for example, orally, parenterally, intravenously, intramuscularly, subcutaneously, intradermally, transdermally, intrathecally, submucosally, sublingually, rectally, vaginally, etc.

Once the compositions have been prepared, they may be combined with pharmaceutically acceptable excipients to form a pharmaceutical composition. As would be appreciated by one of skill in this art, the excipients may be chosen based on the route of administration as described below, the agent being delivered, and the time course of delivery of the agent.

Pharmaceutical compositions described herein and for use in accordance with the embodiments described herein may include a pharmaceutically acceptable excipient. As used herein, the term “pharmaceutically acceptable excipient” means a non-toxic, inert solid, semi-solid or liquid filler, diluent, encapsulating material or formulation auxiliary of any type. Some examples of materials which can serve as pharmaceutically acceptable excipients are sugars such as lactose, glucose, and sucrose; starches such as corn starch and potato starch; cellulose and its derivatives such as sodium carboxymethyl cellulose, methylcellulose, hydroxypropylmethylcellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen free water; isotonic saline; citric acid, acetate salts, Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions of this invention can be administered to humans and/or to animals, orally, rectally, parenterally, intracisternally, intravaginally, intranasally, intraperitoneally, topically (as by powders, creams, ointments, or drops), bucally, or as an oral or nasal spray.

Liquid dosage forms for oral administration include pharmaceutically acceptable emulsions, microemulsions, solutions, suspensions, syrups, and elixirs. In addition to the active ingredients (i.e., the particles), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3 butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, ethanol, U.S.P., and isotonic sodium chloride solution. In addition, sterile, fixed oils are conventionally employed as a solvent or suspending medium. For this purpose any bland fixed oil can be employed including synthetic mono or diglycerides. In addition, fatty acids such as oleic acid are used in the preparation of injectables.

The injectable formulations can be sterilized, for example, by filtration through a bacteria retaining filter, or by incorporating sterilizing agents in the form of sterile solid compositions which can be dissolved or dispersed in sterile water or other sterile injectable medium prior to use.

Compositions for rectal or vaginal administration may be suppositories which can be prepared by mixing the particles with suitable non irritating excipients or carriers such as cocoa butter, polyethylene glycol, or a suppository wax which are solid at ambient temperature but liquid at body temperature and therefore melt in the rectum or vaginal cavity and release the particles.

Solid dosage forms for oral administration include capsules, tablets, pills, powders, and granules. In such solid dosage forms, the particles are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Solid compositions of a similar type may also be employed as fillers in soft and hard filled gelatin capsules using such excipients as lactose or milk sugar as well as high molecular weight polyethylene glycols and the like.

The solid dosage forms of tablets, dragées, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Dosage forms for topical or transdermal administration of a pharmaceutical composition include ointments, pastes, creams, lotions, gels, powders, solutions, sprays, inhalants, or patches. The particles are admixed under sterile conditions with a pharmaceutically acceptable carrier and any needed preservatives or buffers as may be required. Ophthalmic formulation, ear drops, and eye drops are also possible.

The ointments, pastes, creams, and gels may contain, in addition to the compositions of this invention, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the compositions of this invention, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compositions in a proper medium. Absorption enhancers can also be used to increase the flux of the compound across the skin. The rate can be controlled by either providing a rate controlling membrane or by dispersing the compositions in a polymer matrix or gel.

In other embodiments, the stabilized compositions of the invention are loaded and stored in prefilled syringes and cartridges for patient-friendly autoinjector and infusion pump devices.

Kits for use in preparing or administering the compositions are also provided. A kit for forming compositions may include any solvents, solutions, buffer agents, acids, bases, salts, targeting agent, etc. needed in the composition formation process. Different kits may be available for different targeting agents. In certain embodiments, the kit includes materials or reagents for purifying, sizing, and/or characterizing the resulting compositions. The kit may also include instructions on how to use the materials in the kit. The one or more agents (e.g., pharmaceutically active agent) to be contained within the composition are typically provided by the user of the kit.

Kits are also provided for using or administering the compositions. The compositions may be provided in convenient dosage units for administration to a subject. The kit may include multiple dosage units. For example, the kit may include 1-100 dosage units. In certain embodiments, the kit includes a week supply of dosage units, or a month supply of dosage units. In certain embodiments, the kit includes an even longer supply of dosage units. The kits may also include devices for administering the compositions. Exemplary devices include syringes, spoons, measuring devices, etc. The kit may optionally include instructions for administering the compositions (e.g., prescribing information).

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As disclosed herein, the terms “composition” and “formulation” are used interchangeably.

In some embodiments, article A comprises a liquid pharmaceutical composition comprising RNA formulated in a lipid nanoparticle, liposome, or lipoplex; wherein the article has a shelf-life of at least three months when stored at a temperature of greater than 0° C. and less than or equal to 10° C.; and wherein the amount is greater than or equal to (1+the fraction of the RNA that would degrade in the liquid pharmaceutical composition over the shelf-life of the article)×(an individual dose of the liquid pharmaceutical composition).

According to some embodiments of article A, the article comprises a total amount of full length RNA, and the total amount of full length RNA is greater than or equal to (1+the fraction of the full length RNA that would degrade in the liquid pharmaceutical composition over the shelf-life of the article)×(an individual dose of the full length RNA)×(the number of individual doses of the liquid pharmaceutical composition in the article).

In certain embodiments, article AA comprises a liquid pharmaceutical composition comprising RNA formulated in a lipid nanoparticle, liposome, or lipoplex; wherein the article has a shelf-life of at least three months when stored at a temperature of greater than 0° C. and less than or equal to 10° C.; and wherein the article comprises a total amount of full length RNA, and the total amount of full length RNA is greater than or equal to (1+the fraction of the full length RNA that would degrade in the liquid pharmaceutical composition over the shelf-life of the article)×(an individual dose of the full length RNA)×(the number of individual doses of the liquid pharmaceutical composition in the article.

In certain embodiments of articles A and/or AA, the article further comprises a label, suggesting an amount of the liquid pharmaceutical composition to be administered to a subject.

In some embodiments of articles A and/or AA, the article comprises a vial, a syringe, a cartridge, an infusion pump, and/or a light protective container. In certain embodiments of articles A and/or AA, the amount is greater than or equal to 1.05×(an individual dose of the liquid pharmaceutical composition) and/or greater than or equal to 1.2×(an individual dose of the liquid pharmaceutical composition). In some embodiments of articles A and/or AA, the amount is less than or equal to 2.00×(an individual dose of the liquid pharmaceutical composition).

According to some embodiments of article A and/or AA, the amount of the liquid pharmaceutical composition is a volume of the liquid pharmaceutical composition and the dose is a mass of RNA in a unit volume.

In accordance with certain embodiments of articles A and/or AA, the RNA is encapsulated within the lipid nanoparticle, liposome, or lipoplex. According to some embodiments of articles A and/or AA, the lipid nanoparticle, liposome, or lipoplex comprises a lipid nanoparticle. In certain embodiments of articles A and/or AA, the lipid nanoparticle, liposome, or lipoplex comprises a liposome. In some embodiments of articles A and/or AA, the lipid nanoparticle, liposome, or lipoplex comprises a lipoplex.

According to certain embodiments, article B comprises a liquid pharmaceutical composition comprising an RNA encoding an antigen formulated in a lipid carrier housed in a container; wherein the container comprises a total amount of RNA and wherein the total amount of RNA includes 40%-95% intact RNA and 5%-60% RNA that is less than full length RNA. In some embodiments the composition comprises 40%-95% pure RNA. In some embodiments of article B, the percentage of intact RNA is greater than or equal to 15%+the percentage of the RNA that would degrade in the liquid pharmaceutical composition over a shelf-life of the article. In certain embodiments of article B, the article comprises at least 5% more intact RNA than a minimum therapeutically effective dose of the intact RNA.

In some embodiments of article B, the total amount of RNA includes 40%-80% intact RNA and 20%-60% RNA that is less than full length RNA. In certain embodiments of article B, the total amount of RNA includes 40%-70% intact RNA and 30%-60% RNA that is less than full length RNA. In accordance with some embodiments of article B, the total amount of RNA includes 60%-80% intact RNA and 20%-40% RNA that is less than full length RNA. According to certain embodiments of article B, the total amount of RNA includes 70%-95% intact RNA and 5%-30% RNA that is less than full length RNA. In some embodiments of article B, the total amount of RNA includes 75-90% intact RNA and 10%-25% RNA that is less than full length RNA. In certain embodiments of article B, the total amount of RNA includes 75-80% intact RNA and 20%-25% RNA that is less than full length RNA.

In some embodiments of article B, the article further comprises a label on the container, wherein the label identifies a number of individual doses of the liquid pharmaceutical composition housed in the container, an amount of each individual dose of the liquid pharmaceutical composition to be administered to a subject, and an effective dose of RNA within the liquid pharmaceutical composition within each individual dose, wherein the container comprises a total amount of RNA, wherein the total amount of RNA has a value of at least the number of individual doses in the container times 5% greater than the amount of the effective dose of RNA within each individual dose.

In certain embodiments, article C comprises a liquid pharmaceutical composition comprising an RNA formulated in a lipid carrier housed in a container; wherein the container comprises a total amount of RNA, wherein the total amount of RNA has a value of at least a number of individual doses in the container times 5% greater than the amount of the effective dose of RNA within each individual dose.

According to some embodiments of article C, the container comprises a total amount of full length RNA, wherein the total amount of full length RNA is at least the number of individual doses in the container times 5% greater than the amount of the effective dose of full length RNA within each individual dose.

In some embodiments of article C, the article further comprises a label on the container, wherein the label identifies the number of individual doses of the liquid pharmaceutical composition housed in the container, an amount of each individual dose of the liquid pharmaceutical composition to be administered to a subject, and an effective dose of RNA within the liquid pharmaceutical composition within each individual dose.

According to certain embodiments of articles B and/or C, the total amount of RNA has a value of at least the number of individual doses in the container times 20% greater than the amount of the effective dose of RNA within each individual dose. In accordance with some embodiments of articles B and/or C, the total amount of RNA has a value of at least the number of individual doses in the container times 30% greater than the amount of the effective dose of RNA within each individual dose. In some embodiments or articles B and/or C, the total amount of RNA has a value of less than or equal to the number of individual doses in the container times 100% greater than the amount of the effective dose of RNA within each individual dose.

In accordance with certain embodiments of articles B and/or C, the article has a shelf-life of at least one month when stored at a temperature of greater than 0° C. and less than or equal to 10° C. According to some embodiments of articles B and/or C, the article has a shelf-life of at least three months when stored at a temperature of greater than 0° C. and less than or equal to 10° C.

In some embodiments of articles A, AA, B and/or C, the article has a shelf-life of at least one month when stored at a temperature of 5° C. In certain embodiments of articles A, AA, B, and/or C, the article has a shelf-life of at least three months when stored at a temperature of 5° C. According to some embodiments of articles A, AA, B and/or C, at least 40% of the total amount of RNA in the liquid pharmaceutical composition is intact if stored for three months at 5° C. In accordance with certain embodiments of articles A, AA, B and/or C at least 50% of the total amount of RNA in the liquid pharmaceutical composition is intact if stored for three months at 5° C. In some embodiments of articles A, AA, B and/or C, at least 60% of the total amount of RNA in the liquid pharmaceutical composition is intact if stored for three months at 5° C. In certain embodiments of articles A, AA, B and/or C, at least 70% of the total amount of RNA in the liquid pharmaceutical composition is intact if stored for three months at 5° C. In accordance with some embodiments of articles A, AA, B and/or C, at least 90% of the total amount of RNA in the liquid pharmaceutical composition is intact if stored for three months at 5° C.

In certain embodiments of articles B and/or C, the container comprises a light protective container. In some embodiments of articles B and/or C, the container comprises a vial, a syringe, a cartridge, and/or an infusion pump. According to some embodiments or articles B and/or C, the RNA is encapsulated within the lipid carrier.

In some embodiments of articles A, AA, B and/or C, the label indicates that the article should not be stored at the glass transition temperature of the liquid pharmaceutical composition. In certain embodiments of articles A, AA, B and/or C, the label indicates that the article should not be stored at a temperature of less than or equal to −20° C. and greater than or equal to −50° C. According to some embodiments of articles A, AA, B and/or C, the label indicates that the article should not be stored at a temperature of less than or equal to −30° C. and greater than or equal to −35° C. In accordance with certain embodiments of articles A, AA, B and/or C, the lipid carrier comprises a lipid nanoparticle.

According to certain embodiments of B and/or C, the lipid carrier comprises a liposome. In some embodiments of B and/or C, the lipid carrier comprises a lipoplex.

In certain embodiments of articles A, AA, B and/or C, the individual dose of the liquid pharmaceutical composition is the individual dose needed to produce a therapeutically effective amount of a protein in the subject. In accordance with some embodiments of articles A, AA, B and/or C, the individual dose of the liquid pharmaceutical composition is the individual dose approved by the FDA to stimulate an antigen specific immune response in the subject.

According to some embodiments of articles A, AA, B and/or C, the lipid nanoparticle comprises a ratio of 20-60% amino lipids, 5-30% phospholipid, 10-55% structural lipid, and 0.5-15% PEG-modified lipid. In accordance with certain embodiments of articles A, AA, B and/or C, the lipid nanoparticle comprises a ratio of 20-60% amino lipids, 5-25% phospholipid, 25-55% structural lipid, and 0.5-15% PEG-modified lipid.

In some embodiments of articles A, AA, B and/or C, the RNA comprises mRNA. In certain embodiments of articles A, AA, B and/or C, the RNA comprises greater than or equal to 400, 500, 1000, 2000, 3000, 4000, 5000, 6000, 7000, 8000, 9000, or 10,000 nucleotides. For example, in some embodiments of articles A, AA, B and/or C, the RNA comprises greater than or equal to 400 nucleotides. According to certain embodiments of articles A, AA, B and/or C, the RNA comprises greater than or equal to 4,000 nucleotides. In accordance with some embodiments of articles A, AA, B and/or C, the RNA comprises less than or equal to 20,000, 15,000, 14,000, 13,000, 12,000, 11,000, 10,000, 9000, 8000, 7000, or 6000 nucleotides. For example, in certain embodiments of articles A, AA, B and/or C, the RNA comprises less than or equal to 10,000 nucleotides. In some embodiments of articles A, AA, B and/or C, the RNA comprises less than or equal to 6,000 nucleotides.

In certain embodiments of articles A, AA, B and/or C, the liquid pharmaceutical composition is formulated in an aqueous solution.

According to certain embodiments of articles A, AA, B and/or C, the mRNA encodes an antigen. In some embodiments of articles A, AA, B and/or C, the antigen is an infectious disease antigen. In certain embodiments of articles A, AA, B and/or C, the infectious disease is caused by or associated with a virus. In some embodiments of articles A, AA, B and/or C, the antigen is a cancer antigen. According to certain embodiments of articles A, AA, B and/or C, the cancer antigen is a personalized cancer antigen. According to some embodiments of articles A, AA, B and/or C, the mRNA encodes a therapeutic protein.

In some embodiments of articles A, AA, B and/or C, the article comprises a total amount of the liquid pharmaceutical composition, wherein the total amount is 1.25×10 individual doses x (an individual dose of the liquid pharmaceutical composition).

In certain embodiments, pharmaceutical composition A comprises mRNA encapsulated in a lipid nanoparticle, wherein the composition comprises a total amount of intact mRNA that is greater than an effective amount of intact mRNA, and wherein the composition comprises at least the effective amount of the intact mRNA after storage of the composition for a period of time. In accordance with some embodiments of pharmaceutical composition A, the total amount of intact mRNA decreases in the composition after storage of the composition for the period of time. According to certain embodiments of pharmaceutical composition A, the total amount of intact mRNA is calculated to account for degradation of the mRNA during the storage of the composition for the period of time.

According to some embodiments of pharmaceutical composition A, the degradation is from transesterification of the intact mRNA. In accordance with certain embodiments of pharmaceutical composition A, the degradation is greater than or equal to 5%, greater than or equal to 7%, greater than or equal to 8%, greater than or equal to 9%, greater than or equal to 10%, or greater than or equal to 12% of the total mRNA in the composition per month. In certain embodiments of pharmaceutical composition A, the period of time is greater than or equal to 1 month, greater than or equal to 2 months, greater than or equal to 3 months, greater than or equal to 6 months, or greater than or equal to 9 months. In some embodiments of pharmaceutical composition A, the storage is at a temperature of from about 0° C. to about 10° C., such as at about 5° C.

In accordance with some embodiments of pharmaceutical composition A, the total amount of intact mRNA is at least 40%, such as at least 50%, at least 55%, at least 60%, at least 63%, at least 65%, at least 70%, at least 75%, at least 80%, at least 85%, at least 90%, or at least 95% of the total mRNA in the composition. In certain embodiments of pharmaceutical composition A, the effective amount of intact mRNA is at least about 15%, such as at least about 18%, at least about 20%, at least about 25%, at least about 30%, at least about 35%, at least about 40%, at least about 45%, at least about 50%, or at least about 55% of the total mRNA in the composition. In some embodiments of pharmaceutical composition A, the pharmaceutical composition comprises at least 50% intact mRNA of the total mRNA in the composition following storage of the composition for 3 months at about 5° C. In certain embodiments of pharmaceutical composition A, the effective amount comprises at least 5 micrograms of the intact mRNA, such as at least 10 micrograms, at least 20 micrograms, at least 30 micrograms, at least 40 micrograms, at least 50 micrograms, at least 60 micrograms, at least 70 micrograms, at least 80 micrograms, at least 90 micrograms, at least 100 micrograms, at least 125 micrograms, or at least 150 micrograms of the intact mRNA.

In some embodiments, a container (such as a vial, a syringe, a cartridge, an infusion pump, and/or a light protective container) comprises pharmaceutical composition A.

In certain embodiments of articles A, AA, B, and/or C, the pharmaceutical composition comprises pharmaceutical composition A.

In some embodiments, method A of filling an article comprises adding RNA formulated in a lipid nanoparticle, liposome, or lipoplex to the article to form an amount of a liquid pharmaceutical composition in the article; wherein the amount of RNA is greater than or equal to (1+the fraction of the RNA that would degrade in the liquid pharmaceutical composition over the shelf-life of the article)×(an individual dose of the liquid pharmaceutical composition)×(the number of individual doses in the article).

In accordance with some embodiments of method A, wherein the adding RNA formulated in a lipid nanoparticle, liposome, or lipoplex to the article forms an amount of full length RNA in the article, and wherein the amount of full length RNA is greater than or equal to (1+the fraction of the RNA that would degrade in the liquid pharmaceutical composition over the shelf-life of the article)×(an individual dose of the full length RNA)×(the number of individual doses in the article).

According to certain embodiments of method A, the RNA and/or lipid nanoparticle, liposome, or lipoplex are frozen prior to addition to the article.

In accordance with certain embodiments of method A, the article is stored at a temperature of greater than 0° C. and less than 10° C. for up to 1 year. According to some embodiments of method A, the article is stored at a temperature of greater than 0° C. and less than 10° C. for up to 3 months. In some embodiments of method A, at least 40% of the amount of the RNA in the liquid pharmaceutical composition is intact if stored for three months at a temperature of greater than 0° C. and less than 10° C. In certain embodiments of method A, at least 50% of the amount of the RNA in the liquid pharmaceutical composition is intact if stored for three months at a temperature of greater than 0° C. and less than 10° C.

According to some embodiments of method A, the liquid pharmaceutical composition comprises pharmaceutical composition A.

In accordance with some embodiments of method A, at least 60% of the amount of the RNA in the liquid pharmaceutical composition is intact if stored for three months at a temperature of greater than 0° C. and less than 10° C. In certain embodiments of method A, at least 70% of the amount of RNA in the liquid pharmaceutical composition is intact if stored for three months at a temperature of greater than 0° C. and less than 10° C. According to certain embodiments of method A, at least 75% of the amount of RNA in the liquid pharmaceutical composition is intact if stored for three months at a temperature of greater than 0° C. and less than 10° C. In some embodiments of method A, the temperature is 5° C.

In certain embodiments of method A, the article is not stored at the glass transition temperature of the liquid pharmaceutical composition. In some embodiments of method A, the article is not stored at less than or equal to −20° C. and greater than or equal to −50° C. In accordance with certain embodiments of method A, the article is not stored at less than or equal to −30° C. and greater than or equal to −35° C.

According to certain embodiments of method A, the amount of RNA is greater than or equal to 1.05×(an individual dose of the liquid pharmaceutical composition)×(the number of individual doses in the article). In accordance with some embodiments of method A, the amount of RNA is greater than or equal to 1.2×(an individual dose of the liquid pharmaceutical composition)×(the number of individual doses in the article). In certain embodiments of method A, the amount of RNA is less than or equal to 2.00×(an individual dose of the liquid pharmaceutical composition)×(the number of individual doses in the article).

In some embodiments of method A, the article comprises a vial, a syringe, a cartridge, an infusion pump, and/or a light protective container.

In accordance with certain embodiments of method A, the amount is 1.25×10 individual doses×(an individual dose of the liquid pharmaceutical composition).

According to some embodiments, method B of delivering an effective dose of an RNA to a subject, comprises administering a liquid pharmaceutical composition comprising an RNA encoding a protein formulated in a lipid carrier to a subject, wherein a total dose of the RNA is administered to the subject, and wherein the total dose of RNA administered to the subject is at least 5% greater than the effective dose of the RNA. In certain embodiments of method B, the liquid pharmaceutical composition comprises pharmaceutical composition A.

In certain embodiments of method B, the total dose of RNA administered to the subject is at least 20% greater than the effective dose of the RNA. In accordance with some embodiments of method B, the total dose of RNA administered to the subject is at least 30% greater than the effective dose of the RNA. In some embodiments of method B, the total dose of the RNA administered to the subjected is less than or equal to 100% greater than the effective dose of the RNA.

According to certain embodiments of method B, the lipid carrier comprises a lipid nanoparticle, liposome, or lipoplex.

In certain embodiments of methods A and/or B, the RNA is encapsulated within the lipid nanoparticle, liposome, or lipoplex in the liquid pharmaceutical composition. In some embodiments of methods A and/or B, the lipid nanoparticle, liposome, or lipoplex comprises a lipid nanoparticle. In certain embodiments of methods A and/or B, the lipid nanoparticle, liposome, or lipoplex comprises a liposome. According to some embodiments of methods A and/or B, the lipid nanoparticle, liposome, or lipoplex comprises a lipoplex.

In accordance with certain embodiments of methods A and/or B, the individual dose of the liquid pharmaceutical composition is the individual dose needed to produce a therapeutically effective amount of a protein in the subject. According to some embodiments of methods A and/or B, the individual dose of the liquid pharmaceutical composition is the individual dose approved by the FDA to stimulate an antigen specific immune response in the subject.

In accordance with some embodiments of methods A and/or B, the lipid nanoparticle comprises a ratio of 20-60% amino lipids, 5-30% phospholipid, 10-55% structural lipid, and 0.5-15% PEG-modified lipid. In certain embodiments of methods A and/or B, the lipid nanoparticle comprises a ratio of 20-60% amino lipids, 5-25% phospholipid, 25-55% structural lipid, and 0.5-15% PEG-modified lipid.

In some embodiments of methods A and/or B, the RNA comprises mRNA. In certain embodiments of methods A and/or B, the RNA comprises greater than or equal to 400, 500, 1000, 2000, 3000, 4000, 5000, 6000, 7000, 8000, 9000, or 10,000 nucleotides. For example, in some embodiments of methods A and/or B, the RNA comprises greater than or equal to 400 nucleotides. In accordance with certain embodiments of methods A and/or B, the RNA comprises greater than or equal to 4,000 nucleotides. According to some embodiments of methods A and/or B, the RNA comprises less than or equal to 20,000, 15,000, 14,000, 13,000, 12,000, 11,000, 10,000, 9000, 8000, 7000, or 6000 nucleotides. For example, in certain embodiments of methods A and/or B, the RNA comprises less than or equal to 10,000 nucleotides. In accordance with some embodiments of methods A and/or B, the RNA comprises less than or equal to 6,000 nucleotides.

In certain embodiments of methods A and/or B, the liquid pharmaceutical composition is formulated in an aqueous solution.

In accordance with some embodiments of methods A and/or B, the mRNA encodes an antigen. In some embodiments of methods A and/or B, the antigen is an infectious disease antigen. In certain embodiments of methods A and/or B, the infectious disease is caused by or associated with a virus. In certain embodiments of methods A and/or B, the antigen is a cancer antigen. According to some embodiments of methods A and/or B, the cancer antigen is a personalized cancer antigen. In accordance with certain embodiments of methods A and/or B, the mRNA encodes a therapeutic protein.

In some embodiments, method C of compensating for transesterification of mRNA in a composition comprising the mRNA encapsulated by a lipid nanoparticle comprises preparing the composition with increased mRNA purity as compared to an mRNA purity that will be present in the composition after storage of the composition, such that the amount of mRNA present in the composition after storage will comprise an effective amount of the mRNA. According to some embodiments of method C, the composition comprises pharmaceutical composition A.

The following examples are intended to illustrate certain embodiments of the present invention, but do not exemplify the full scope of the invention. Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever.

### EXAMPLES

### Example 1

This example describes the degradation of mRNA in lipid nanoparticle formulations when stored for 3 months at 5° C. This example demonstrates that the main mechanism of degradation of mRNA in lipid nanoparticle formulations at these conditions is trans-esterification (rather than hydrolysis).

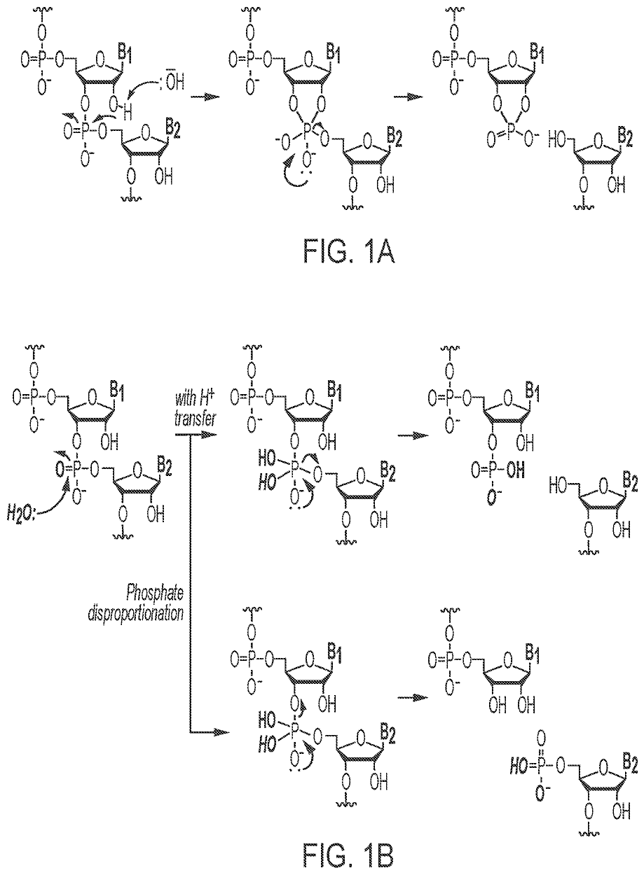

In transesterification, 2′-hydroxyl moieties of ribose rings along the mRNA's backbone nucleophilically attack their adjacent phosphates to form cyclic pentavalent phosphorus intermediates. These transient intermediates then collapse, either leading to 2′-5′-phosphodiester linkages (backbone isomerization, which is not discussed here), or leading to strand scission, resulting in fragment strands that are terminated by 2′-3′-cyclic phosphates on their 3′-ends (see FIG. 1A). On the other hand, in hydrolysis, water nucleophilically attacks the 3′-5′-phosphodiester linkages in a bimolecular fashion to form linearized pentavalent phosphorus intermediates, which would then collapse and disproportionate the phosphate groups to either the 3′- or 5′-ends of the fragments (see FIG. 1B). Thus, in transesterification reactions resulting in strand scission, the phosphate groups always reside at the 3′-ends, while in hydrolysis reactions, the phosphate groups can reside at both the 5′-ends and the 3′-ends.

The degradation of mRNA was studied for LNP formulations with two different types of mRNA (one that encodes a first viral antigen and one that encodes for a second different viral antigen), to demonstrate that the mechanism of degradation is independent of sequence. This was studied using a 3′-RACE+/−PNK workflow (3′-rapid amplification of cDNA ends+/−polynucleotide kinase), which allowed for rapid profiling of the 3′-end sites. mRNA fragments were ligated with a sequence-defined, 5′-adenylated DNA adaptor oligonucleotide at their 3′-ends using thermostable T4 ligase; the ligated DNA-RNA hybrid strands were then subjected to library prep and NGS sequencing on a MiSeq (Illumina).

It should be noted that this workflow only applies to mRNA fragments that are 3′-terminated as hydroxyl groups. If the mRNA fragments are 3′-phosphate protected—as in the case of transesterification-derived fragments—these phosphates must be cleaved prior to sequencing. In this example, this was achieved by incorporating a polynucleotide kinase (PNK)-mediated phosphate removal step. Thus, by comparing the number of sequencing reads in PNK-treated vs. non-PNK-treated samples, it could be determined whether the 3′-termini of RNA fragments were phosphorylated or remained as hydroxyls.

If transesterification were the major strand cleavage mechanism, the 3′-termini of RNA fragments would be expected to be primarily phosphorylated, and it would be expected to (1) detect a lot more sequence reads in the PNK-treated sample than in the non-PNK-treated samples, and (2) detect minimal sequence reads in the non-PNK-treated samples. On the other hand, if hydrolysis were the major strand cleavage mechanism, the backbone phosphate groups would be expected to be disproportioned to either the 5′- or 3′-end of the fragments, and hence some portion of fragment 3′-termini would be expected to remain as unphosphorylated 3′-hydroxyls. Thus, it would be expected to detect some abundance of sequencing reads in the non-PNK-treated samples as well.

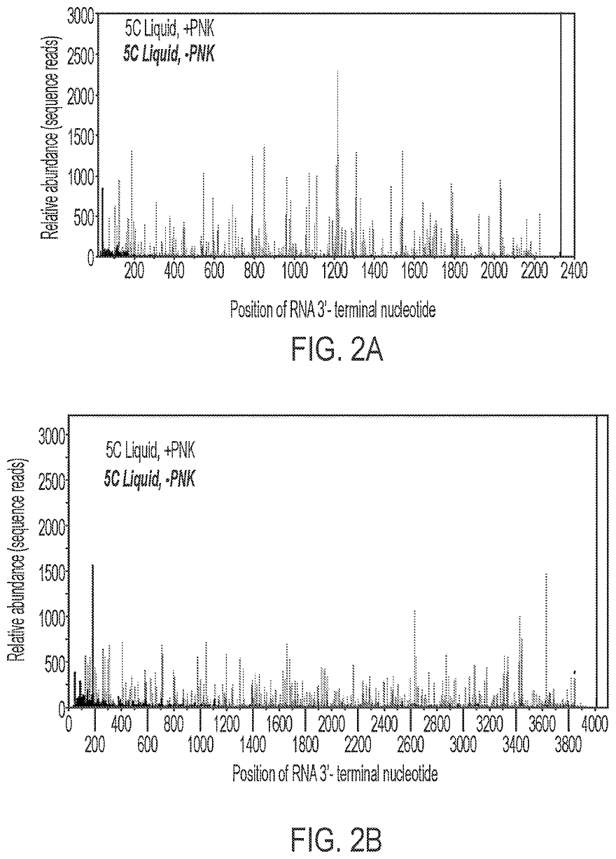

Liquid LNP formulations were analyzed after storage for 3 months at 5° C., as shown in FIG. 2A (a formulation comprising mRNA that encodes a viral antigen) and FIG. 2B (a formulation comprising mRNA that encodes a different viral antigen). The X-axis denotes the position at which RNA fragment ligation to the sequence-defined DNA adaptor occurred, which is in turn indicative of the 3′-ends of the RNA fragments. The Y-axis corresponds to the number of detected sequence reads that have 3′-ends corresponding to the respective nucleotide. In both FIGS. 2A and 2B, which show with PNK and without PNK, sequence reads were detected almost exclusively in the PNK-treated samples, and very little sequence reads were detected in the non-PNK-treated samples except for the full-length product (which is hydroxyl-terminated). This observation suggested that most RNA fragments had 3′-ends that were phosphorylated, and very few fragments were 3′-terminated as unprotected hydroxyls.

These findings indicate that both mRNAs underwent strand scission by a transesterification mechanism, and this this is the predominant mechanism of degradation of mRNA regardless of sequence.

### Example 2

This example describes the relationship between degradation of mRNA and the number of nucleotides of the mRNA. This example demonstrates that the percentage of degraded mRNA generally increases as the number of nucleotides in the mRNA increases.

As demonstrated in Example 1, mRNA degradation predominantly takes place via transesterification resulting in an integral full-length parent mRNA breaking into smaller fragments. Transesterification is a random event and can occur at any site along the mRNA backbone. Therefore, relative to shorter mRNAs, longer mRNAs have a higher probability of incurring strand breakage and are mechanistically predicted to degrade faster.

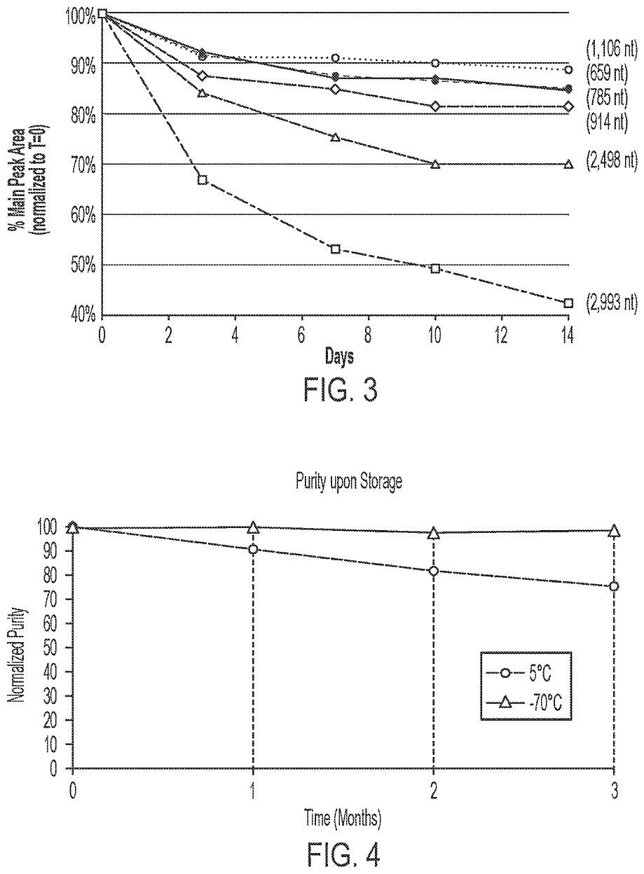

Six formulations with mRNAs with different numbers of nucleotides (i.e., 659, 785, 914, 1,106, 2,498, and 2,993 nucleotides) were monitored by a size-based RP-HPLC purity method over 14 days stored at 40° C. (see FIG. 3). FIG. 3 demonstrates that the percentage of degraded mRNA generally increased as the number of nucleotides in the mRNA increased.

Without wishing to be bound by theory, it is believed that, if discrepancies are observed, they could be due to co-elution of some longer mRNA fragments with the integral full-length mRNA in some instances. Nevertheless, overall, the data demonstrate that the percentage of degraded mRNA generally increases as the number of nucleotides in the mRNA increases.

### Example 3

This example describes the amount of degradation observed when an LNP formulation comprising mRNA (that has over 4,000 nucleotides) is stored at 5° C. and −70° C. As shown in FIG. 4, the degradation of the mRNA was higher at 5° C. than at −70° C. As shown in FIG. 4, the degradation rate at 5° C. was determined to be approximately 8% degradation per month at 5° C.

### Example 4

This example evaluates the in vivo response of an LNP formulation comprising mRNA (that encodes a viral antigen) after partial degradation due to simulation of long term storage via application of heat.

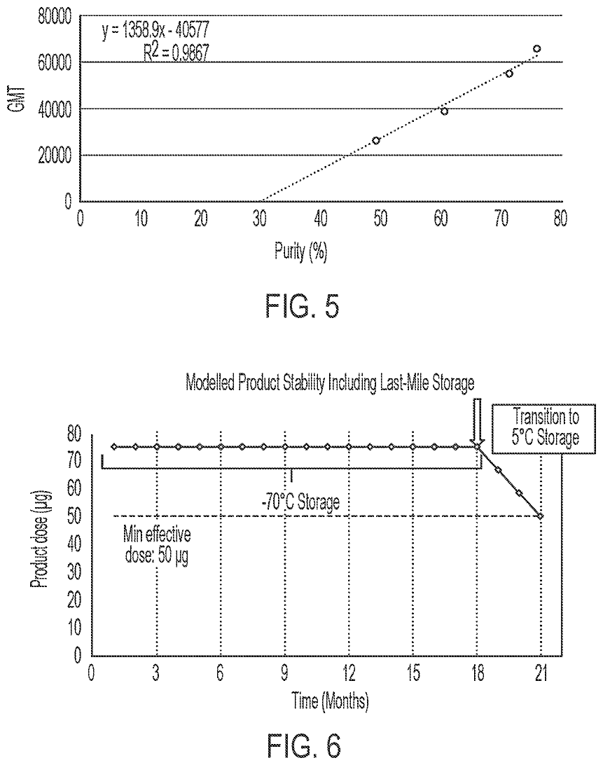

12 female 8-week old BALB/C mice were injected on day 1 and day 22 with 2 μg of the same LNP formulations with various amounts of degradation. The formulations had been treated with heat to simulate various amounts of time stored at 5° C.: 0 months (76% mRNA purity), 4 months (71% mRNA purity), 14 months (61% mRNA purity), and 26 months (49% mRNA purity). As shown in FIG. 5, the geometric mean titers produced in the subjects decreased linearly with decreasing purity. This demonstrates that the purity of the mRNA may affect the geometric mean titers produced in the subject.

### Example 5

This example describes the balance between stability of an article and commercial supply of the article.

Pharmaceutical products, including vaccines, degrade over time, which ultimately results in a loss of activity. An understanding of the mechanisms of product degradation is critical to managing the overall shelf-life of the product.

The proposed storage of the product is −70° C. to maximize product shelf-life, however it is understood that this may not be suitable for commercialization and supply in certain geographical regions particularly in lower middle, or lower income countries where cold-chain storage and supply is challenging. An alternative was developed in which shelf life is managed through the determination of the minimum potency requirement (minimum effective dose), determination of the degradation rate, and then provision of additional product in the vial to account for degradation at higher storage temperatures. The exact amount included will be dependent upon the final dose selected in clinical trials, and the amount of time required at non-frozen storage conditions. It is expected that the selected dose will be sufficiently low, such that the inclusion of additional drug in the vial will not significantly impact cost or manufacturing complexity.

This provides significant supply chain and storage flexibility for the product, which includes a stable product at −70° C. combined with the opportunity to include additional material to permit storage at 5° C., nominally for 3 months, which is consistent with industry expectations for vaccines, including in lower income countries.

A driver towards a commercially acceptable vaccine product is the alignment of the overall product stability and shelf-life at the intended storage condition with the requirements of manufacturing, distribution and administration of the product. For many vaccines, particularly those utilizing live attenuated viral vectors, degradation of the product upon storage is expected, even when stored frozen. Similarly, for all nucleic-acid based vaccines, some degradation of the product during storage is expected, particularly at elevated temperatures. This degradation however is not expected to be limiting to the commercial suitability or utility of the proposed vaccine.

Fundamental characterization of product degradation, as described in Example 1, has driven a mechanistic understanding which has ultimately led to process improvements and tighter product control. Broadly speaking, the mechanisms of degradation in the lipid nanoparticle (LNP)-mRNA products can be categorized as either being driven by physical (e.g. particle integrity) or chemical (mRNA strand integrity or lipid degradation) processes. As for many biological products, there are a number of critical quality (analytical) attributes for the product, and by extension a number of these are considered to be limiting for the product if they drop below a specified threshold. The advances in process and storage understanding resulted in a particle that is generally physically stable, however storage around the glass transition (e.g., −20° C. to −40° C.) of the product may increase physical instability. The main limiting factor for stability of the vaccine has been determined to be due to chemical degradation, specifically breakage of the mRNA strands in an aqueous environment. Through a series of detailed studies (see Example 1), it was determined that this degradation is driven by a transesterification reaction. The approach to determining shelf-life of the product was therefore based on the mRNA construct purity. As full-length mRNA is required for activity, degradation/breakage of the mRNA strand will render it inactive.

The rate of mRNA degradation was dependent upon temperature, as shown in FIG. 4, the vaccine product showed negligible product degradation at −70° C., which provides flexibility in manufacturing. This allows for use of bulk freezing technology, for example, for storage of materials prior to vial filling. At 5° C., mRNA degradation was observed as shown in FIG. 4.

As −70° C. may not be preferred as a commercial storage or distribution condition, particularly in regions with limited cold-chain (frozen) infrastructure and depot storage, refrigerated (5° C.) cold-chain supply is likely to be preferred. The rate of degradation of mRNA will be used to determine the effective amount of vaccine required in the product. This will be achieved in clinical studies in which both the dose required to engender the desired immunological response, and the overall safety profile will be assessed.

The approach therefore is to provide additional material in the vials by increasing vial mRNA content (μg) to account for degradation. A schematic of the product degradation/shelf life and additional content considerations is shown in FIG. 6. It is likely that the vaccine product will require a dose below 200 micrograms, permitting additional material to be included without significantly impacting the commercial suitability of the product. The upper dose that can be selected will be determined from the safety data obtained during ongoing clinical studies.

The non-lyophilized product and mRNA-LNP platform are suitable for commercialization and supply in real-world situations, particularly in lower middle, or lower income countries where cold-chain storage and supply (including at health care provider premises) may not be robust. As it is probable that the minimum effective dose will be less than 200 μg and possibly less than 100 μg (data pending), additional material included in the drug product vial will be possible and will permit flexibility in supply, an appropriate shelf-life, and last-mile storage and supply of the product.

This product has significant supply chain and storage flexibility, namely a stable product at −70° C. combined with the opportunity to include additional material to permit storage at 5° C., nominally for 3 months, which is consistent with industry expectations for vaccines.

### Example 6

This example demonstrates the determination of the glass transition temperature of several compositions comprising mRNA in lipid nanoparticles with varying levels of Tris and sucrose.

As described above, the glass transition temperature is the temperature at which an amorphous substance (e.g., sucrose) transitions from a hard and relatively brittle (“glassy”) state into a rubbery or viscous state. Without wishing to be bound by theory, it is believed that product stability is well maintained in the vitrified state as product mobility that may generate deleterious chemical reactions or aggregation events are essentially ceased.

The glass transition temperature (Tg′) of compositions were measured by modulated Differential Scanning Calorimetry (mDSC). Tg′ was measured using the reversing heat flow to isolate the Tg′ from non-reversing events, such as crystalline melts and enthalpic relaxations/reorganizations caused by disordered freezing.

As shown in Table 1, as the relative concentration of Tris to sucrose increased in the compositions, the Tg′ decreased.

TABLE 1
Measured Tg’ for Tris-Sucrose Systems

| Tris (mM) | Sucrose (g/L) | Tg’ (° C.) |
|---|---|---|
| 0 | 50 | −32.6 |
| 0 | 123 | −31.5 |
| 0 | 200 | −30.5 |
| 25 | 50 | −36.2 |
| 25 | 123 | −33.6 |
| 25 | 200 | −32.3 |
| 50 | 50 | −38.5 |
| 50 | 123 | −34.7 |
| 50 | 200 | −33.4 |
| 100 | 50 | −42.1 |
| 100 | 123 | −36.4 |
| 100 | 200 | −35.0 |

### Example 7

This prophetic example demonstrates a method of filling an article, in accordance with certain embodiments.

A nucleic acid (e.g., mRNA) is combined with a lipid carrier (e.g., LNP) to form an amount of a liquid pharmaceutical composition in an article (e.g., a vial), wherein the nucleic acid (e.g., mRNA) is formulated in the lipid carrier (e.g., LNP). The amount of liquid pharmaceutical composition in the article is demonstrated in Table 2.

The fourth and fifth columns of Table 2 are appropriate for various combinations of shelf-life and degradation rate. For example, the fourth column of Table 2 is appropriate for an article with a 3 month shelf-life (e.g., at 5° C.) and a degradation rate of −8.3% per month. Similarly, the fourth column of Table 2 would also be appropriate for an article with a 2 month shelf-life and a degradation rate of 12.5% per month, or an article with a 6 month shelf-life and a degradation rate of ˜4.1% per month.

Similarly, the fifth column of Table 2 is appropriate for an article with a 3 month shelf-life (e.g., at 5° C.) and a degradation rate of 10% per month, as well as an article with a 2 month shelf-life and a degradation rate of 15% per month, or an article with a 6 month shelf-life and a degradation rate of 5% per month.

TABLE 2
Liquid Pharmaceutical Composition Amounts in Articles

| | | | | Alternative |
|---|---|---|---|---|
| | | | Amount of liquid | amount of liquid |
| | Number | Amount of liquid | pharmaceutical | pharmaceutical |
| Individual | of Doses | pharmaceutical | composition in | composition in |
| Dose | in | composition in | article | article |
| (micrograms) | Article | article (micrograms) | (micrograms) | (micrograms) |
| 25 | 10 | 10* 25 * (1 + | 312.5 | 325 |
| | | fraction of nucleic | | |
| | | acid that would | | |
| | | degrade over shelf- | | |
| | | life) | | |
| 25 | 20 | 20 * 25 * (1 + | 625 | 650 |
| | | fraction of nucleic | | |
| | | acid that would | | |
| | | degrade over shelf- | | |
| | | life) | | |
| 25 | 50 | 50 * 25 * (1 + | 1,562.5 | 1,625 |
| | | fraction of nucleic | | |
| | | acid that would | | |
| | | degrade over shelf- | | |
| | | life) | | |
| 100 | 10 | 10 * 100 * (1 + | 1,250 | 1,300 |
| | | fraction of nucleic | | |
| | | acid that would | | |
| | | degrade over shelf- | | |
| | | life) | | |
| 100 | 20 | 20 * 100 * (1 + | 2,500 | 2,600 |
| | | fraction of nucleic | | |
| | | acid that would | | |
| | | degrade over shelf- | | |
| | | life) | | |
| 100 | 50 | 50 * 100 * (1 + | 6,250 | 6,500 |
| | | fraction of nucleic | | |
| | | acid that would | | |
| | | degrade over shelf- | | |
| | | life) | | |
| 250 | 10 | 10 * 250 * (1 + | 3,125 | 3,250 |
| | | fraction of nucleic | | |
| | | acid that would | | |
| | | degrade over shelf- | | |
| | | life) | | |
| 250 | 20 | 20 * 250 * (1 + | 6,250 | 6,500 |
| | | fraction of nucleic | | |
| | | acid that would | | |
| | | degrade over shelf- | | |
| | | life) | | |
| 250 | 50 | 50 * 250 * (1 + | 15,625 | 16,250 |
| | | fraction of nucleic | | |
| | | acid that would | | |
| | | degrade over shelf- | | |
| | | life) | | |

This example demonstrates that, in some instances, mRNA vaccines are effective at low purity levels.

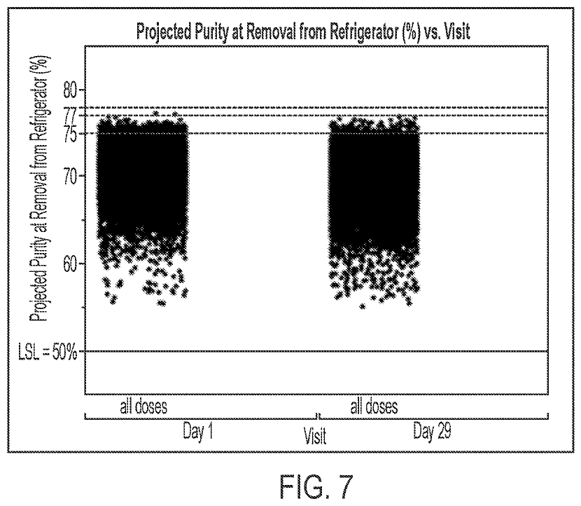

The purity of mRNA (i.e., that has over 4,000 nucleotides) in 15,000 vaccine doses (each with 100 micrograms of mRNA) was determined. After this determination was made, the 15,000 doses were kept in the refrigerator (approximately 5° C.) for various periods of time (up to approximately 85 days) before administration to human subjects. The rate of degradation for this mRNA under these conditions was determined. The percentage purity of the mRNA at the time of administration was calculated based on the initial measured purity, the amount of time each dose was kept in the refrigerator, and the determined rate of degradation under those conditions. The y-axis of FIG. 7 shows the calculated purity when removed from the refrigerator (which, in this case, was also the time of administration). As shown in FIG. 7, doses ranging from under 55% projected purity to over 77% projected purity were administered to human subjects on day 1, and then doses that again ranged from under 55% projected purity to 77% or higher projected purity were administered to the same human subjects on day 29.

Further, it was determined that the efficacy of the vaccine was not directly related to purity alone, but instead was directly related to the amount of intact mRNA administered. For example, a 50 microgram dose of mRNA with 100% intact mRNA (or 100% purity) would provide 50 micrograms of intact mRNA while a 100 microgram dose of mRNA with 50% intact mRNA (or 50% purity) would also provide 50 micrograms of intact mRNA, and both would provide a similar immune response since they have the same amount of intact mRNA.

This relationship was further explored by increasing the total amount of mRNA administered and decreasing the purity (e.g., to 46%, 30%, and 18% purity). It was determined that equivalent immune responses could be achieved with vaccines with these lower purities when the total amount of mRNA was increased, such that the total amount of intact mRNA delivered was equivalent.

Thus, this example demonstrates that it is the amount of intact mRNA administered that affected the efficacy of the studied mRNA vaccine rather than the purity of the mRNA.

### Example 9

This example studied the minimum amount of intact mRNA needed to ensure effective vaccination of human subjects in order to determine the shelf-life of the vaccine and/or the starting mRNA purity needed to ensure that at least the minimum amount of intact mRNA would be administered throughout the shelf-life of the vaccine.

Multiple amounts of intact mRNA were administered to human subjects and the efficacy of the vaccine was studied. It was determined that the efficacy of the vaccine plateaued as the amount of intact mRNA increased, such that there was no observed benefit for efficacy of increasing the amount of intact mRNA beyond the plateau amount. Accordingly, for purposes of this example, it was determined that at least this plateau amount of intact mRNA should be delivered in each dose throughout the shelf-life of the vaccine to ensure no variations in vaccine efficacy. Accordingly, the shelf-life of the vaccine was determined for individual samples taking into consideration the starting mRNA purity, the rate of degradation of the mRNA in specific storage conditions, and the plateau amount of intact mRNA. From this, a general shelf-life for the vaccine was established. Once the general shelf-life was established, the minimum starting mRNA purity needed in the vaccine was determined by taking into consideration the shelf-life, the rate of degradation of the mRNA in specific storage conditions, and the plateau amount of intact mRNA.

It was determined that the presence of degraded mRNA did not affect safety or efficacy of the vaccine.

Thus, this example demonstrates how the starting mRNA purity, the shelf-life of the vaccine, and the final amount of intact mRNA (e.g., the plateau amount) interact with one another. For example, it was determined that to extend the shelf-life (or include storage conditions where degradation is accelerated), the plateau amount of intact mRNA could still be administered at any point throughout the shelf-life if the mRNA purity in the starting product was increased.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. Each possibility represents a separate embodiment of the present invention.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An article, comprising:
a liquid pharmaceutical composition comprising RNA formulated in a lipid nanoparticle, liposome, or lipoplex; and
a label, suggesting an amount of the liquid pharmaceutical composition to be administered to a subject;
wherein the article has a shelf-life of at least three months when stored at a temperature of greater than 0° C. and less than or equal to 10° C.; and
wherein the amount is greater than or equal to (1+the fraction of the RNA that would degrade in the liquid pharmaceutical composition over the shelf-life of the article)×(an individual dose of the liquid pharmaceutical composition).

2. The article of claim 1, wherein the article comprises a total amount of full length RNA, and the total amount of full length RNA is greater than or equal to (1+the fraction of the full length RNA that would degrade in the liquid pharmaceutical composition over the shelf-life of the article)×(an individual dose of the full length RNA)×(the number of individual doses of the liquid pharmaceutical composition in the article).

3. An article, comprising:
a liquid pharmaceutical composition comprising RNA formulated in a lipid nanoparticle, liposome, or lipoplex;
wherein the article has a shelf-life of at least three months when stored at a temperature of greater than 0° C. and less than or equal to 10° C.; and
wherein the article comprises a total amount of full length RNA, and the total amount of full length RNA is greater than or equal to (1+the fraction of the full length RNA that would degrade in the liquid pharmaceutical composition over the shelf-life of the article)×(an individual dose of the full length RNA)×(the number of individual doses of the liquid pharmaceutical composition in the article).

4. The article of any of the preceding claims, wherein the article comprises a vial, a syringe, a cartridge, an infusion pump, and/or a light protective container.

5. The article of any preceding claims, wherein the amount is greater than or equal to 1.05×(an individual dose of the liquid pharmaceutical composition), such as greater than or equal to 1.2×(an individual dose of the liquid pharmaceutical composition).

6. The article of any preceding claim, wherein the RNA is encapsulated within the lipid nanoparticle, liposome, or lipoplex.

7. The article of any preceding claim, wherein the lipid nanoparticle, liposome, or lipoplex comprises a lipid nanoparticle.

8. An article, comprising:
a liquid pharmaceutical composition comprising an RNA encoding an antigen formulated in a lipid carrier housed in a container; wherein the container comprises a total amount of RNA and wherein the total amount of RNA includes 40%-95% intact RNA and 5%-60% RNA that is less than full length RNA.

9. The article of claim 8, wherein the percentage of intact RNA is greater than or equal to 15%+the percentage of intact RNA that would degrade in the liquid pharmaceutical composition over a shelf-life of the article.

10. The article of claim 8 or 9, wherein the article comprises at least 5% more intact RNA than an effective dose of the intact RNA.

11. An article, comprising:
a liquid pharmaceutical composition comprising an RNA formulated in a lipid carrier housed in a container; and
a label on the container, wherein the label identifies a number of individual doses of the liquid pharmaceutical composition housed in the container, an amount of each individual dose of the liquid pharmaceutical composition to be administered to a subject, and an effective dose of RNA within the liquid pharmaceutical composition within each individual dose,
wherein the container comprises a total amount of RNA, wherein the total amount of RNA has a value of at least the number of individual doses in the container times 5% greater than the amount of the effective dose of RNA within each individual dose.

12. The article of claim 11, wherein the container comprises a total amount of full length RNA, wherein the total amount of full length RNA is at least the number of individual doses in the container times 5% greater than the amount of the effective dose of full length RNA within each individual dose.

13. The article of any one of claims 8-12, wherein the article has a shelf-life of at least three months when stored at a temperature of greater than 0° C. and less than or equal to 10° C.

14. The article of any one of claims 8-13, wherein the RNA is encapsulated within the lipid carrier.

15. The article of any one of claims 8-14, wherein the lipid carrier comprises a lipid nanoparticle.

16. The article of any preceding claim, wherein the RNA comprises mRNA.

17. The article of any preceding claim, wherein the RNA comprises greater than or equal to 400, 500, 1000, 2000, 3000, 4000, 5000, 6000, 7000, or 8000 nucleotides.

18. The article of any preceding claim, wherein the RNA comprises less than or equal to 15,000, 14,000, 13,000, 12,000, 11,000, 10,000, 9000, 8000, 7000, or 6000 nucleotides.

19. The article of any preceding claim, wherein the liquid pharmaceutical composition is formulated in an aqueous solution.

20. A pharmaceutical composition comprising mRNA encapsulated in a lipid nanoparticle, wherein the composition comprises a total amount of intact mRNA that is greater than an effective amount of intact mRNA, and wherein the composition comprises at least the effective amount of the intact mRNA after storage of the composition for a period of time.

21. The pharmaceutical composition of claim 20, wherein the total amount of intact mRNA decreases in the composition after storage of the composition for the period of time.

22. The pharmaceutical composition of claim 20 or 21, wherein the total amount of intact mRNA is calculated to account for degradation of the intact mRNA during the storage of the composition for the period of time.

23. The pharmaceutical composition of claim 22, wherein the degradation is from transesterification of the intact mRNA.

24. The pharmaceutical composition of claim 22 or 23, wherein the degradation is greater than or equal to 5%, greater than or equal to 7%, greater than or equal to 8%, greater than or equal to 9%, greater than or equal to 10%, or greater than or equal to 12% of the total mRNA in the composition per month.

25. The pharmaceutical composition of any one of claims 20-24, wherein the period of time is greater than or equal to 1 month, greater than or equal to 2 months, greater than or equal to 3 months, greater than or equal to 6 months, or greater than or equal to 9 months.

26. The pharmaceutical composition of any one of claims 20-25, wherein the storage is at a temperature of from about 0° C. to about 10° C., such as at about 5° C.

27. The pharmaceutical composition of any one of claims 20-26, wherein the total amount of intact mRNA is at least 40%, such as at least 50%, at least 55%, at least 60%, at least 63%, at least 65%, at least 70%, at least 75%, at least 80%, at least 85%, at least 90%, or at least 95% of the total mRNA in the composition.

28. The pharmaceutical composition of any one of claims 20-27, wherein the effective amount of intact mRNA is at least about 15%, such as at least about 18%, at least about 20%, at least about 25%, at least about 30%, at least about 35%, at least about 40%, at least about 45%, at least about 50%, or at least about 55% of the total mRNA in the composition.

29. The pharmaceutical composition of any one of claims 20-28, wherein the pharmaceutical composition comprises at least 50% intact mRNA of the total mRNA in the composition following storage of the composition for 3 months at about 5° C.

30. The pharmaceutical composition of any one of claim 20-29, wherein the effective amount of intact mRNA comprises at least 5 micrograms of the intact mRNA, such as at least 10 micrograms, at least 20 micrograms, at least 30 micrograms, at least 40 micrograms, at least 50 micrograms, at least 60 micrograms, at least 70 micrograms, at least 80 micrograms, at least 90 micrograms, at least 100 micrograms, at least 125 micrograms, or at least 150 micrograms of the intact mRNA.

31. A container (such as a vial, a syringe, a cartridge, an infusion pump, and/or a light protective container) comprising the pharmaceutical composition of any one of claims 20-30.

32. An article of any one of claims 1-19, wherein the pharmaceutical composition is the pharmaceutical composition of any one of claims 20-30.

33. A method of filling an article, comprising:
adding RNA formulated in a lipid nanoparticle, liposome, or lipoplex to the article to form an amount of a liquid pharmaceutical composition in the article;
wherein the amount is greater than or equal to (1+the fraction of the RNA that would degrade in the liquid pharmaceutical composition over the shelf-life of the article)×(an individual dose of the liquid pharmaceutical composition)×(the number of individual doses in the article).

34. The method of claim 33, wherein the adding RNA formulated in a lipid nanoparticle, liposome, or lipoplex to the article forms an amount of full length RNA in the article, and wherein the amount of full length RNA is greater than or equal to (1+the fraction of the full length RNA that would degrade in the liquid pharmaceutical composition over the shelf-life of the article)×(an individual dose of the full length RNA)×(the number of individual doses in the article).

35. The method of any one of claims 33-34, wherein the RNA and/or lipid nanoparticle are frozen prior to addition to the article.

36. The method of any one of claims 33-35, wherein the article is stored at a temperature of greater than 0° C. and less than 10° C. for up to 1 year.

37. The method of any one of claims 33-36, wherein at least 40% of the amount of the RNA in the liquid pharmaceutical composition is intact if stored for three months at a temperature of greater than 0° C. and less than 10° C.

38. The method of any one of claims 33-37, wherein the liquid pharmaceutical composition comprises the pharmaceutical composition of any one of claims 20-30.

39. The method of any one of claims 34-38, wherein the lipid nanoparticle, liposome, or lipoplex comprises a lipid nanoparticle.

40. A method of delivering an effective dose of an RNA to a subject, comprising;
administering a liquid pharmaceutical composition comprising an RNA encoding a protein formulated in a lipid carrier to a subject, wherein a total dose of the RNA is administered to the subject, and wherein the total dose of RNA administered to the subject is at least 5% greater than an effective dose of the RNA.

41. The method of claim 40, wherein the lipid carrier comprises a lipid nanoparticle.

42. A method of compensating for transesterification of mRNA in a composition comprising the mRNA encapsulated by a lipid nanoparticle, the method comprising preparing the composition with increased mRNA purity as compared to an mRNA purity that will be present in the composition after storage of the composition, such that the amount of mRNA present in the composition after storage will comprise an effective amount of the mRNA.

43. The method of claim 42, wherein the composition comprises the pharmaceutical composition of any one of claims 20-30.
